SEQUENCE LISTING (4 sequences)

SEQ ID NO: 1 - DNA, 939 bp, Pichia pastoris
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 2 - DNA, 247 bp, Pichia pastoris
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgaga

SEQ ID NO: 3 - DNA, 489 bp, Bos taurus
ctcattgtcacccagaccatgaagggcctggatatccagaaggtggcggggacttggtactccttggccatggcggccagcgacatctccctgctggacgcccagagtgcccccctgagagtgtatgtggaggagctgaagcccacccctgagggcgacctggagatcctgctgcagaaatgggagaacggtgagtgtgctcagaagaagatcattgcagaaaaaaccaagatccctgcggtgttcaagatcgatgccttgaatgagaacaaagtccttgtgctggacaccgactacaaaaagtacctgctcttctgcatggagaacagtgctgagcccgagcaaagcctggcctgccagtgcctggtcaggaccccggaggtggacgacgaggccctggagaaattcgacaaagccctcaaggccctgcccatgcacatccggctgtccttcaacccaacccagctggaggagcagtgccacatctag

SEQ ID NO: 4 - protein, 162 aa, Bos taurus
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

(19) United States
(12) Patent Application Publication
(10) Pub. No.: US 2024/0215599 A1
(43) Pub. Date: Jul. 4, 2024

(54) DAIRY ANALOGUES COMPRISING BETA-LACTOGLOBULIN

(71) Applicant: RE-MILK LTD., Ness Ziona (IL)

(72) Inventors: Ori Cohavi, Ness Ziona (IL); Liam Sharon, Ness Ziona (IL); Matanel Tassa, Ness Ziona (IL)

(21) Appl. No.: 18/558,292

(22) PCT Filed: May 10, 2022
(86) PCT No.: PCT/IL2022/050484

§ 371 Date: Oct. 31, 2023

Related U.S. Application Data
(60) Provisional application No. 63/187,798, filed on May 12, 2021.
(60) Provisional application No. 63/224,133, filed on Jul. 21, 2021.
(60) Provisional application No. 63/289,640, filed on Dec. 15, 2021.

(51) Int. Cl. A23C 21/10; A23C 3/02; A23C 20/00; A23C 21/08

(52) U.S. Cl. CPC A23C 21/10; A23C 3/02; A23C 20/00; A23C 21/08

(57) ABSTRACT

The present invention provides transgenic yeast microorganisms useful for production of high amounts of recombinant beta-lactoglobulin (BLG), and isolates of the BLG obtained from the transgenic yeasts. The invention further provides dairy analogue food products comprising BLG, such as recombinant BLG, as the main milk protein, and methods for producing such dairy analogue food products.

### FIELD OF THE INVENTION

The present invention relates to transgenic yeast useful for the production of a high quantity of recombinant beta-lactoglobulin (BLG), to isolates of the BLG obtained from the transgenic yeasts, as well as to dairy analogue food products in which BLG, such as recombinant BLG, is the main milk protein. The present invention is further related to methods of production of such dairy analogue food products.

### BACKGROUND OF THE INVENTION

Production of transgenic Pichia pastoris (P. pastoris) is unpredictable, as evident by Schwarzhans et al. Microb Cell Fact (2016) 15:84, which concludes that methods for transformation of P. pastoris led to a multitude of unintended and sometimes detrimental integration events, lowering total productivity. Tae-Rak Kim et al. (Protein Engineering vol. 10 no. 11 pp. 1339-1345, 1997) refers to high-level expression of bovine beta-lactoglobulin in Pichia pastoris and characterization of its physical properties.

Optimization of the fermentation indicators to maximize production of the recombinant protein (many fermentation variables, not necessarily additive) is not trivial at all, as discussed in Liu, W. C. et al. (Scaling-up Fermentation of Pichia pastoris to demonstration-scale using new methanol-feeding strategy and increased air pressure instead of pure oxygen supplement. Sci. Rep. 6, 18439; doi: 10.1038/srep18439 (2016)).

The first and most fundamental stage of making any type of cheese is controlled coagulation. The process of milk coagulation is the destabilization of the casein micelles, which flocculate and aggregate to form a gel made of the soluble milk components. Coagulation can be induced by acidification, enzymatic reaction, or a combination of the two. During the process of turning milk into cheese, the fat and casein are concentrated, while the other components of milk (including water) are removed. The removed water contains lactose, globular proteins, minerals, and fat, generally referred to as “Whey”.

Whey proteins are isolated from whey and are often dried into whey protein concentrate (WPC) powder. WPC is a source for different proteins and lactose. Whey proteins are small, globular, and soluble. They are sensitive to heat and acid and do not react with chymosin. These attributes make them unsuitable for traditional cheese making. The only dairy product, traditionally made from whey proteins (or whey), is Ricotta. Ricotta is made by heating the whey to a temperature above 85° C. and instant acidification using edible acid causing a rapid protein aggregation. That fast aggregation pushes most of the soluble molecules away from the clotted proteins. At that high temperature, no LAB can be added for aroma development.

It was previously demonstrated that whey protein aggregation happened to a much larger extent when all 3 major proteins are present (beta-lactoglobulin, alpha-lactalbumin and bovine serum albumin). The lactose fraction in the WPC is the preferred LAB's carbon source which is the major precursor for lactic and organic acid production.

Typically, whey proteins do not coagulate. Thus, producing traditional casein-based dairy products using only whey proteins in general, and specifically using predominantly BLG, was not suggested, let alone could be obtained, thus far. Further, it was revealed that it is an almost an impossible mission to produce traditional high-rheology dairy products (yogurt, ice cream) without considerable amounts of stabilizers (Madiha Tasneem et al., (2014, Stabilizers: Indispensable Substances in Dairy Products of High Rheology, Critical Reviews in Food Science and Nutrition, 54:7, 869-879, DOI: 10.1080/10408398.2011.614702).

There is a continuous need for the development of commercially viable methods for the preparation of alternative proteins and dairy analogue food products.

### SUMMARY OF THE INVENTION

It is well known that obtaining stains of microorganisms producing high, commercially sustainable amounts of proteins of interest is not always trivial and sometimes requires some inventive thinking. The present application provides a transgenic Pichia pastoris cell adapted to maximize production of recombinant beta-lactoglobulin B (rBLG) protein. It has further been found, according to the teaching of the present invention, that the specific BG11 mutant cell, transformed with pJAG and/or pJAN expression vectors carrying the native (non-codon-optimized) rBLG open reading frame (ORF), more specifically containing a high number of copies, e.g. 7-20 copies of the rBLG ORFs, is especially suitable for the production of high amounts of rBLG, and is useful for the production of a protein isolate with a high degree of purity.

An additional aspect of the present invention relates to production of dairy analogue food products, and more specifically, dairy analogue food products which are based primarily on BLG as the main milk protein. In a typical process of preparing traditional dairy products, casein proteins, which are the abundant proteins in milk, are coagulated via enzymatic coagulation by enzymes such as rennet or chymosin. BLG accounts for less than 10% of all bovine milk protein. One of the advantages of BLG is that it is a source of a single pure protein (with only traces of fats and minerals, and no sugars). Due to its nature, BLG does not react with chymosin and is very sensitive to heat. Therefore, until the present invention, BLG was not considered a valuable protein that may be used for the preparation of dairy analogue products. In particular, BLG was not used, or suggested to be used, in the preparation of dairy analogue products, as the sole whey protein (e.g., without alpha-lactalbumin), as the sole milk protein (e.g., without caseins), or as the sole protein in general.

It has been found by the present inventors that when applying a traditional cheese-making protocol on an artificially-produced cheese composition comprising BLG as a main milk protein, the composition pre-maturely coagulates, thus preventing the cheese-analogues product from being produced with the desired properties. Therefore, as a first step, the inventors found a way to overcome the fact that a reconstituted liquid made from BLG denatures at pasteurization heat, and causes an unwanted increase of viscosity. The rapid increase of viscosity clogs the pasteurizer and/or interferes with pumping the sample from the pasteurizer to the incubation tank, and prevents adding the rest of the ingredients (like LAB). The present invention shows for the first time that BLG may be controllably-coagulated and therefore used as the main protein in dairy analogue food products. As such, the present invention provides methods for coagulation of BLG, methods for preparation of dairy analogue food products and subsequently the dairy analogue food products themselves.

As the dairy analogue food products may be produced in a completely controllable way, such products, if desired, would not comprise any components obtained from an animal source. Moreover, the dairy analogue food products have the same organoleptic and/or rheologic properties as the dairy food product to which they are analogues.

In addition, the inventors of the present invention found a replacement for lactose. Lactose is the natural energy source for the LAB and has a low sweetness index (the kind of sweetness associated with most cheese). However, due to lactose-intolerance of certain populations, the use of lactose may be problematic. The inventors have developed a tailor-made alternative. It should be understood that using the wrong sugar(s) might contribute to non-LAB-dependent acidification or insufficient acidification, and that using the wrong dosage of sugar(s) might lead to insufficient acidification or to the final product being overly-sweet.

According to one aspect, the present invention provides a transgenic Pichia pastoris (P. pastoris) cell, comprising from 10 to 20 copies of a nucleic acid sequence encoding a recombinant beta-lactoglobulin B (rBLG) protein, wherein the P. pastoris cell is a slow-methanol-utilization (Mut^{S}) derivative of P. pastoris. Thus, in some embodiments, the present invention provides a transgenic Pichia pastoris (P. pastoris) cell, comprising from 10 to 20 copies of a nucleic acid molecules encoding a recombinant beta-lactoglobulin B (rBLG) protein, wherein the P. pastoris cell is a slow-methanol-utilization (Mut^{S}) derivative of P. pastoris.

According to some embodiments, each one of the nucleic acid sequences encoding rBLG is operably linked to an AOX1 transcription promoter and an AOX1 transcription terminator and is located between the AOX1 promoter and the AOX1 transcription terminator. According to some embodiments, the transgenic P. pastoris cell comprises from 10 to 18, from 12 to 16, from 13 to 15, or about 14 copies of the sequence encoding the rBLG protein.

According to another aspect, the present invention provides a recombinant BLG (rBLG) protein isolate, produced by and isolated from the transgenic P. pastoris cells of the present invention.

According to yet another aspect, the present invention provides a dairy analogue food product, comprising the rBLG protein isolate as described herein.

According to one aspect, the present invention provides a method comprising:
(i) pasteurizing a composition comprising a BLG protein, wherein the composition is substantially devoid of coagulation minerals;
(ii) optionally, cooling down the composition of step (i); and
(iii) adding a coagulation mineral to the composition of step (ii), if present, or to the composition of step (i), if step (ii) is absent, thereby coagulating the BLG protein,
wherein BLG constitutes at least 51 wt % of the total milk-protein content of the composition, and
wherein the composition comprises at least about 0.75 wt % BLG.

According to one aspect, the present invention provides a method of preparing a curd comprising a coagulated beta-lactoglobulin B (BLG), the method comprising:
(i) pasteurizing a composition comprising a BLG protein, wherein the composition is substantially devoid of coagulation minerals;
(ii) optionally, cooling down the composition of step (i); and
(iii) adding a coagulation mineral to the composition of step (ii), if present, or to the composition of step (i), if step (ii) is absent, thereby coagulating the BLG protein,
wherein BLG constitutes at least 51 wt % of the total milk-protein content of the curd, and
wherein the curd comprises at least about 0.75 wt % BLG.

According to one aspect, the present invention provides a method of preparing a dairy analogue food product comprising a coagulated beta-lactoglobulin B (BLG), the method comprises:
(i) pasteurizing a composition comprising a BLG protein, wherein the composition is substantially devoid of coagulation minerals;
(ii) optionally, cooling down the composition of step (i); and
(iii) adding a coagulation mineral to the composition of step (ii), if present, or to the composition of step (i), if step (ii) is absent, thereby coagulating the BLG protein,
wherein BLG constitutes at least 51 wt % of the total milk-protein content of the dairy analogue food product, and wherein the dairy analogue food product comprises at least about 0.75 wt % BLG.

According to some embodiments, the method comprises cooling down the composition of step (i). According to some embodiments, the coagulation mineral comprises one or more salts of a mineral selected from the group consisting of calcium, magnesium, phosphorus, potassium, selenium, and zinc. According to one embodiment, the coagulation mineral is calcium. According to one embodiment, the coagulation mineral salt is calcium chloride. According to some embodiments, the resulted dairy analogue food product comprises from about 0.75 to about 8 wt % of BLG. According to one embodiment, the resulted dairy analogue food product comprises from about 0.0015 to about 0.35 wt % of a coagulation mineral.

According to some embodiments, the dairy analogue food product comprises from about 1.5 to about 8 wt % BLG, and step (iii) comprises adding from about 0.0015 to about 0.035 wt % of a coagulation mineral. According to other embodiments, the dairy analogue food product comprises from about 1.5 to about 4.5 wt % BLG, and step (iii) comprises adding from about 0.001 to about 0.07 wt % of the coagulation mineral. According to a further embodiment, the dairy analogue food product comprises from about 1.5 to about 3.5 wt % BLG, and step (iii) comprises adding from about 0.001 to about 0.1 wt % of a coagulation mineral. According to an alternative embodiment, the dairy analogue food product comprises from about 1 to about 3 wt % BLG, and step (iii) comprises adding from about 0.1 to about 0.2 wt % of a coagulation mineral.

According to some embodiments, the method further comprises subjecting the dairy analogue food product to acidification to prepare an acidified dairy analogue product. According to some embodiments, the method further comprises subjecting the dairy analogue food product to saltation to prepare a salted dairy analogue product. According to some embodiments, the method further comprises subjecting the dairy analogue food product to smoothening to prepare a smooth dairy analogue product.

According to an alternative embodiment, the method further comprising subjecting the dairy analogue food product to the following steps:
(i) subjecting the dairy analogue food product to a lactic acidification to produce an acidified dairy analogue food product; and/or
(ii) adding a flavoring salt to the dairy analogue food product to produce a salted dairy analogue food product; and/or
(iii) smoothing the dairy analogue food product to produce a smooth dairy analogue food.

According to another aspect, the present invention provides a method of preparing a dairy analogue food product comprising a coagulated BLG protein, the method comprising:
(i) pasteurizing a composition comprising a BLG protein, wherein the composition is substantially devoid of coagulation minerals;
(ii) optionally, cooling down the composition of step (i); and
(iii) acidifying the composition of step (i) or of step (ii), thereby coagulating the BLG protein;
wherein BLG constitutes at least 51 wt % of the total milk-protein content of the dairy analogue food product, and wherein the dairy analogue food product comprises at least about 2 wt % BLG.

According to some embodiments, coagulating the BLG protein in step (iii) results in the dairy analogue food product. According to some embodiments, the resulted dairy analogue food product comprises from about 1.5 to about 8 wt % of BLG.

According to any one of the above aspects and embodiments, the acidification is performed by adding lactic acid bacteria to the dairy analogue food product and fermenting. According to other embodiments, the acidification is performed by adding an acid.

According to another aspect, the present invention provides a dairy analogue food product prepared according to the method according to any one of the above aspects and embodiments. According to some embodiments, the dairy analogue food product is selected from the group consisting of a cream cheese analogue, a Ricotta cheese analogue, an ice cream analogue, and yogurt analogue.

The present invention further provides pre-mix compositions that are useful in preparation of dairy analogue food products.

According to one aspect, the present invention provides a cream cheese analogue pre-mix composition, comprising:
(i) from about 35 to about 80 wt % BLG;
(ii) from about 15 to about 45 wt % of a sugar; and
(iii) from about 0.5 to about 45 wt % of a stabilizer;
wherein the pre-mix composition is substantially devoid of coagulation minerals, and wherein BLG constitutes at least 51 wt % of the total milk-protein content of the pre-mix composition.

According to some embodiments, the cream cheese analogue pre-mix composition comprises (i) from about 35 to about 50 wt % BLG; (ii) from about 15 to about 25 wt % of a sugar, and (iii) from about 25 to about 45 wt % of a stabilizer.

According to some embodiments, the cream cheese analogue pre-mix composition comprises (i) from about 35 to about 50 wt % BLG; (ii) from about 17 to about 25 wt % of a Sugar; and (iii) from about 30 to about 45 wt % of a stabilizer.

According to some embodiments, the cream cheese analogue pre-mix composition comprises (i) from about 35 to about 55 wt % BLG; (ii) from about 10 to about 30 wt % of a sugar; and (iii) from about 20 to about 50 wt % of a stabilizer.

According to some embodiments, the cream cheese analogue pre-mix composition comprises (i) from about 30 to about 50 wt % BLG; (ii) from about 10 to about 30 wt % of a sugar; and (iii) from about 30 to about 50 wt % of a stabilizer.

According to some embodiments, the cream cheese analogue pre-mix composition comprises (i) from about 65 to about 85 wt % BLG; (ii) from about 10 to about 30 wt % of a sugar, and (iii) from about 0.5 to about 2 wt % of a stabilizer.

According to some embodiments, the cream cheese analogue pre-mix composition comprises (i) from about 70 to about 80 wt % BLG; (ii) from about 20 to about 25 wt % of a sugar; and (iii) from about 0.5 to about 3 wt % of a stabilizer.

According to another aspect, the present invention provides a method of preparing a cream cheese analogue comprising a coagulated BLG, the method comprising:
(i) mixing (a) water, (b) from about 5 to about 15 wt % of the cream cheese analogue premix composition as described hereinabove, or the content thereof, and (c) a lipid;
(ii) pasteurizing the composition of step (i);
(iii) optionally, cooling down the composition of step (ii);
(iv) adding a coagulation mineral to the composition of step (ii) or step (iii), thereby coagulating the BLG protein; and
(v) subjecting the composition of step (iv) to an acidification;
(vi) optionally, adding a flavoring salt to the composition of step (v); and
(vii) optionally, smoothing the composition of step (iv) or step (v), wherein BLG constitutes at least 51 wt % of the total milk-protein content of the cream cheese analogue.

According to some embodiments, the method comprises the step (vi). According to some embodiments, the method comprises the step (vii). According to some embodiments, the method comprises steps (vi) and (vii). According to some embodiments, the composition of step (i) is a homogenized composition.

According to some embodiments, the present invention provides a cream cheese analogue, prepared by the method as described hereinabove.

According to another aspect, the present invention provides a cream cheese analogue, comprising:
(i) from about 3 to about 10 wt % BLG;
(ii) from about 1 to about 5 wt % of a sugar;
(iii) optionally, from about 0 to about 5 wt % of a stabilizer;
(iv) from about 15 to about 35 wt % of a lipid;
(v) from about 0.01 to about 3 wt % of a coagulation mineral;
(vi) optionally, from about 0.5 to about 1.5 wt % of a flavoring salt; and
(vii) water up to 100 wt %;
wherein BLG constitutes at least 51 wt % of the total milk-protein content of the cream cheese analogue.

According to some embodiments, the cream cheese analogue comprises from about 0 to about 5 wt % of a stabilizer. According to some embodiments, the cream cheese analogue comprises from greater than 0 to about 5 wt % of a stabilizer.

According to some embodiments, the BLG is coagulated. According to some embodiments, the cream cheese analogue has at least one organoleptic and/or rheologic property of a corresponding dairy cream cheese selected from the group consisting of appearance, glossiness, consistency, structure, thickness, flavor, taste, and penetration strength. According to some embodiments, the cream cheese analogue of the present invention has the appearance, glossiness, consistency, structure, thickness, flavor, taste, and penetration strength of a corresponding dairy cream cheese.

According to another aspect, the present invention provides a method of preparing a Ricotta cheese analogue comprising a coagulated BLG, the method comprising:
(i) dissolving the cream cheese analogue pre-mix composition of the present invention in water and pasteurizing same;
(ii) optionally, cooling down the composition of step (i); and
(iii) adding a coagulation mineral to the composition of step (ii), if present or to the composition of step (i), if step (ii) is absent, thereby coagulating the BLG protein;
   wherein BLG constitutes at least 51 wt % of the total milk-protein content of the Ricotta cheese analogue.

According to some embodiments, the method comprises step (ii).

According to some embodiments, the present invention provides a Ricotta cheese analogue, prepared by the above define method.

According to one aspect, the present invention provides a Ricotta cheese analogue, comprising:
(i) from about 3 to about 8 wt % BLG;
(ii) from about 1 to about 5 wt % of a sugar;
(iii) from about 15 to about 35 wt % of a lipid; and
(iv) water up to 100 wt %;
wherein BLG constitutes at least 51 wt % of the total milk-protein content of the Ricotta cheese analogue.

According to some embodiments, the BLG is coagulated. According to some embodiments, the Ricotta cheese analogue has at least one organoleptic and/or rheologic property of a corresponding dairy Ricotta cheese selected from the group consisting of appearance, glossiness, consistency, structure, thickness, flavor, and taste. According to some embodiments, the Ricotta cheese analogue has the appearance, glossiness, consistency, structure, thickness, flavor, and taste of a corresponding dairy Ricotta cheese.

According to another aspect, the present invention provides a yogurt analogue pre-mix composition, comprising:
(i) from about 50 to about 60 wt % BLG;
(ii) optionally, from about 0.8 to about 2 wt % of a coagulation mineral chelator;
(iii) from about 30 to about 50 wt % of a sugar; and
(iv) from about 0.5 to about 3 wt % of a stabilizer;
wherein the pre-mix composition is substantially devoid of coagulation minerals, and
wherein BLG constitutes at least 51 wt % of the total milk-protein content of the pre-mix composition.

According to another aspect, the present invention provides a method of preparing a yogurt analogue comprising a coagulated BLG, the method comprising:
(i) mixing (a) water, (b) from about 5 to about 20 wt % of the yogurt pre-mix composition as described herein or the content thereof, and (c) from about 1 to about 8 wt % of a lipid;
(ii) pasteurizing the composition of step (i);
(iii) optionally, cooling down the composition of step (ii);
(iv) adding a coagulation mineral salt and acidifying the composition of step (ii), if step (iii) is absent, or of step (iii), thereby coagulating the BLG protein; and
(v) adding from about 5 to about 15 wt % of a sugar,
wherein BLG constitutes at least 51 wt % of the total milk-protein content of the yogurt analogue. According to some embodiments, the method comprises the step (iii).

In certain embodiments, step (iv) comprises adding a coagulation mineral salt and lactic acid bacteria to the composition of step (iii), if present, or to the composition of step (ii), if step (iii) is absent, and allowing fermenting until the pH reaches from about 3.5 to about 4.8, thereby coagulating the BLG protein.

According to some embodiments, the present invention provides a yogurt analogue, prepared by the method as described hereinabove.

According to another aspect, the present invention provides a yogurt analogue, comprising:
(i) from about 2 to about 8 wt % BLG;
(ii) optionally, from about 0.08 to about 0.5 wt % of a coagulation mineral chelator;
(iii) from about 2 to about 15 wt % of a sugar;
(iv) from about 0.05 to about 0.3 wt % of a stabilizer;
(v) from about 1 to about 6 wt % of a lipid;
(vi) from about 0.01 to about 0.15 wt % of a coagulation mineral; and
(vii) water up to 100 wt %,
wherein BLG constitutes at least 51 wt % of the total milk-protein content of the yogurt analogue. According to some embodiments, the BLG is coagulated.

According to some embodiments, the yogurt analogue has at least one organoleptic and/or rheologic property of a corresponding dairy yogurt selected from the group consisting of appearance, glossiness, consistency, structure, thickness, flavor, and taste,. According to some embodiments, the yogurt analogue has the appearance, glossiness, consistency, structure, thickness, flavor, and taste, of a corresponding dairy yogurt.

According to another aspect, the present invention provides an ice cream analogue pre-mix composition, comprising:
(i) from about 6 to about 14 wt % BLG;
(ii) from about 80 to about 85 wt % of a sugar;
(iii) optionally, from about 0.2 to about 0.4 wt % of a coagulation mineral;
(iv) from about 5 to about 7 wt % of dietary fibers;
(v) from about 0.5 to about 1.5 wt % of an emulsifier; and
(vi) from about 0.3 to about 1 wt % of a stabilizer;
(vii) wherein BLG constitutes at least 51 wt % of the total milk-protein content of the pre-mix composition. According to some embodiments, the ice cream analogue pre-mix composition comprises from about 0.2 to about 0.4 wt % of a coagulation mineral.

According to another aspect, the present invention provides a method of preparing an ice cream analogue, comprising:
(i) mixing (a) water, (b) from about 20 to about 30 wt % of the ice cream premix composition as described herein or the content thereof, and (c) from about 7 to about 12 wt % of a lipid;
(ii) homogenizing the composition of step (i); and
(iii) pasteurizing the composition of step (ii);
wherein BLG constitutes at least 51 wt % of the total milk-protein content of the ice cream analogue.

According to some embodiments, the present invention provides an ice cream analogue prepared by the method as defined herein.

According to yet another aspect, the present invention provides an ice cream analogue, comprising:
(i) from about 1 to about 4 wt % BLG;
(ii) from about 10 to about 40 wt % of a sugar;
(iii) optionally, from about 0.05 to about 0.15 wt % of a coagulation mineral;
(iv) from about 1.5 to about 2.1 wt % of dietary fibers;
(v) from about 0.15 to about 0.45 wt % of an emulsifier; and
(vi) from about 0.1 to about 0.3 wt % of a stabilizer;
(vii) from about 5 to about 15 wt % of a lipid;
(viii) optionally, from about 0.05 to about 0.2 wt % of Vanilla extract; and
(ix) water up to 100 wt %;
wherein BLG constitutes at least 51 wt % of the total milk-protein content of the ice cream analogue.

According to some embodiments, the BLG is coagulated.

According to some embodiments, the ice cream analogue has at least one organoleptic and/or rheologic property of a corresponding dairy ice cream selected from the group consisting of appearance, glossiness, consistency, structure, thickness, flavor, taste, and overrun. According to some embodiments, the ice cream analogue the appearance, glossiness, consistency, structure, thickness, flavor, taste, and overrun of a corresponding dairy ice cream.

### BRIEF DESCRIPTION OF THE DRAWINGS

FIG. 1 shows qPCR analysis of rBLG ORF(s). Light gray lines are the control primers (1 copy on another gene). Gray lines cluster into 4 groups: far-right are the 1 copy clones, 2^{nd} from the right are the 2 copy clones, 3^{rd} cluster from the right are both the Bg10 and Bg53 clones present in more than 2 copy (including ˜7 copies), and farthest left cluster are BG11 clones (including ˜14 copies, marked with a circle).

FIG. 2 shows SDS-PAGE gel expression of beta-lactoglobulin in three P. pastoris strains (band at ˜18 kDa).

FIG. 3A shows HPLC of the rBLG protein using HPLC RP-C18. FIG. 3B shows size-exclusion chromatography (SEC) HPLC of the rBLG.

FIG. 4A and FIG. 4B show the distribution of random tasters by age and diet preferences, respectively.

FIG. 5 shows the evaluation of the cream cheese and yogurt analogues according to the present invention by random tasters. FIG. 5A shows a general impression, FIG. 5B shows the evaluation of the resemblance of the analogues to corresponding dairy products, FIG. 5C shows the readiness of the random tasters to replace the product they consume with the cream cheese and yogurt analogues of the present invention.

### DETAILED DESCRIPTION OF THE INVENTION

### Transgenic P. Pastoris

According to one aspect, the present disclosure provides a transgenic microorganism transformed with a plurality of copies of a sequence allowing expression of the beta-lactoglobulin (BLG) protein. According to some embodiments, the microorganism is yeast. According to other embodiments, the microorganism is Pichia. According to other embodiments, the microorganism is Pichia pastoris (P. pastoris). The terms “Pichia pastoris” and “Komagataella phaffii” in some embodiments may be used interchangeably. Thus, the present disclosure provides, in accordance with its broadest aspect, a transgenic P. pastoris cell comprising a nucleic acid sequence allowing expression of the beta-lactoglobulin (rBLG) protein.

According to some embodiments, the transgenic P. pastoris comprises a plurality of copies of the nucleic acid sequence encoding a beta-lactoglobulin (rBLG) protein.

The term “beta-lactoglobulin” (BLG) refers to a beta-lactoglobulin that is typically present in cow's milk. As used in the present invention, the term BLG further refers to isoform B of the BLG, i.e., beta-Lactoglobulin B (β-LG B), which is a small protein of 162 amino acids with a molecular mass of 18.2 kDa and optimum pH of 5.2 (UniProt D6QX31). Nevertheless, in some specific embodiments, the term BLG may refer to BLG-A isoform or to a combination of BLG-A and BLG-B.

The term “recombinant” refers to a polynucleotide, polypeptide or protein that does not naturally occur in a host cell. Thus, the BLG expressed in a microorganism such as Pichia pastoris and specifically BG11 is a recombinant BLG, denoted as rBLG. In some occurrences, rBLG may be, as explained above, BLG-B, BLG-A, or a mixture of BLG-A and BLG-B. According to some embodiments, the BLG and rBLG have the amino acid sequence SEQ ID NO: 4.

In some examples, the sequence allowing expression of the rBLG protein comprises at least the rBLG-encoding sequence between an AOX1 promoter and an AOX1 transcription terminator.

In some examples, the transgenic P. pastoris is a slow-methanol-utilization derivative of P. pastoris. According to some embodiments, the P. pastoris is AOX1 depleted P. pastoris. According to some embodiments, the AOX1 depleted P. pastoris of the present invention is of the BG11 strain.

Thus, in some embodiments, the present invention provides a P. pastoris BG11 strain cell comprising a nucleic acid encoding for BLG-B protein. According to some embodiments, the nucleic acid encoding the BLG protein is located between AOX1 promoter and AOX1 terminator.

In one example, the transgenic P. pastoris is a specific BG11 mutant, comprising the rBLG-encoding sequence between AOX1 promoter and AOX1 transcription terminator (both the promoter and the terminator originating from P. pastoris). In some examples, the AOX1 promoter has a sequence as provided by the ACCESSION FN392322 (REGION: 1549743. 1550681).

In some specific examples, the AOX1 promoter has a nucleotide sequence as defined in SEQ ID NO:1.

In some examples, the AOX1 transcription terminator has a nucleotide sequence as provided by ACCESSION FR839631 (REGION: 240891 . . . 241137). In some examples, the AOX1 transcription terminator has a nucleotide sequence as defined in SEQ ID NO:2.

In some examples, the rBLG coding nucleotide sequence has a nucleotide sequence as defined in SEQ ID NO: 3.

An amino acid sequence of the rBLG of the present invention, produced from the disclosed transgenic microorganism is identified as SEQ ID NO:4. According to some embodiments, the rBLG of the present invention is an analogue of BLG having at least 85%, at least 90% or at least 95% sequence identity to SEQ ID NO: 4.

According to some embodiments, the transgenic P. pastoris such as BG11 comprises a nucleic acid molecule comprising, from 5′ to 3′, nucleic acid sequences, SEQ ID NO: 1, 3 and 2.

In some examples, the transgenic P. pastoris comprises a plurality of copies of the nucleic acid encoding the BLG protein, e.g., from about 10 to about 20, from about 12 to about 18, from about 12 to about 16, from about 13 to about 15 or about 14 copies. In some examples, the transgenic BG11 P. pastoris comprises a plurality of copies of the nucleic acid encoding the BLG protein. According to some embodiments, the BG11 P. pastoris comprises from 7 to 20 copies of the nucleic acid encoding the rBLG protein. According to some embodiments, the transgenic BG11 P. pastoris comprises 7, 8, 9, 10, 11, 12, 13, 14, 15, 16, 17, 18, 19, or 20 copies of the nucleic acid encoding the rBLG protein. In some examples, BG11 P. pastoris comprises between about 10 copies and about 20 copies of the nucleic acid encoding the rBLG protein; at times, between about 7 copies and about 15 copies of the nucleic acid encoding the rBLG protein; at times, between 12 copies and about 18 copies of the nucleic acid encoding the rBLG protein; at times, between about 10 copies and 15 copies of the nucleic acid encoding the rBLG protein. In some examples, BG11 P. pastoris comprises between 13 copies and 15 copies of the nucleic acid encoding the rBLG protein. According to some embodiments, the BG11 P. pastoris comprises about 14 copies of the nucleic acid encoding the rBLG protein. According to some embodiments, the nucleic acid encoding the rBLG protein has a nucleic acid sequence SEQ ID NO: 3. According to some embodiments, the nucleic acid encoding the rBLG protein is a homolog of the nucleic acid sequence SEQ ID NO: 3. According to some embodiments, the nucleic acid encoding the rBLG protein is a conservative homolog of the nucleic acid sequence SEQ ID NO: 3. According to some embodiments, the encoded rBLG protein has an amino acid sequence SEQ ID NO: 4. According to some embodiments, the nucleic acid is a variant having at least 85%, at least 90% or at least 95% sequence identity to the original sequence.

The term “nucleic acid” refers to single stranded or double stranded sequence (polymer) of deoxyribonucleotides or ribonucleotides. In addition, the polynucleotide includes analogues of natural polynucleotides, unless specifically mentioned. According to an embodiment, the nucleic acid may be” selected from the group consisting of deoxyribonucleic acid (DNA), ribonucleic acid (RNA), peptide nucleic acid (PNA), locked nucleic acid (LNA), and analogues thereof, but is not limited thereto. The term encompasses DNA, RNA, single stranded or double stranded and chemical modifications thereof. According to some embodiments, the nucleic acid is DNA.

The terms “homolog”, “variant”, “DNA variant”, “sequence variant” and “polynucleotide variant” are used herein interchangeably and refer to a DNA polynucleotide having at least 70% sequence identity to the parent polynucleotide. The variant may include mutations such as deletion, addition or substitution such that the mutations do not change the open reading frame and the polynucleotide encodes a peptide or a protein having substantially similar structure and function as a peptide or a protein encoded by the parent polynucleotide. According to some embodiments, the variants are conservative variants.

The terms “conservative variants”, “conservative homolog” and “functional homolog” are used herein interchangeably and refer to variants in which a change of one or more nucleotides in a given codon position results in no alteration in the amino acid encoded at that position. Thus, the peptide or the protein encoded by the conservative variants has 100% sequence identity to the peptide or the protein encoded by the parent polynucleotide. According to some embodiments, the variant is a conservative variant having at least 75%, at least 80%, at least 85%, at least 90%, at least 95%, at least 98% or at least 99% sequence identity to the parent polynucleotide sequence.

In some examples, the transgenic P. pastoris cell comprises a plurality of copies of ORFs of the rBLG protein. In the context of the present disclosure, when referring to a plurality of copies of the ORFs, it is to be understood to encompass any number of copies between 7 and 20. According to some embodiments, the P. pastoris is BG11 P. pastoris.

As used herein the term “open reading frame”, “ORF”, “protein ORF”, “protein's ORF” and “ORF of protein” are used interchangeably and refer to a DNA sequence translated into an amino acid sequence, which ranges from a translation start codon (e.g., ATG) to a stop codon (e.g., TGA, TAA, TAG). In some examples, the transgenic P. pastoris comprises at least 7 copies of the protein ORF. In some examples, the transgenic P. pastoris comprises at least 8 copies of the protein ORF. In some examples, the transgenic P. pastoris comprises at least 9 copies of the protein ORF. In some examples, the transgenic P. pastoris comprises at least 10 copies of the protein ORF. In some examples, the transgenic P. pastoris comprises at least 11 copies of the protein ORF. In some examples, the transgenic P. pastoris comprises at least 12 copies of the protein ORF. In some examples, the transgenic P. pastoris comprises at least 13 copies of the protein ORF. In some examples, the transgenic P. pastoris comprises at least 14 copies of the protein ORF. In some examples, the transgenic P. pastoris comprises at least 15 copies of the protein ORF. In some examples, the transgenic P. pastoris comprises at least 16 copies of the protein ORF. In some examples, the transgenic P. pastoris comprises at least 17 copies of the protein ORF. In some examples, the transgenic P. pastoris comprises at least 18 copies of the protein ORF. In some examples, the transgenic P. pastoris comprises at least 19 copies of the protein ORF. According to some embodiments, the P. pastoris is BG11 P. pastori. According to some embodiments, the protein is an analogue of BLG having at least 85%, at least 90% or at least 95% sequence identity to the original sequence.

The term “at least” when used in conjunction with a number of ORFs has the meaning of the value or more. According to the present invention, the term at least X ORFs has the highest limit of 30 ORFs.

In some examples, the transgenic P. pastoris comprises at most 20 copies of the protein ORF. In some examples, the transgenic P. pastoris comprises at most 19 copies of the protein ORF. In some examples, the transgenic P. pastoris comprises at most 18 copies of the protein ORF. In some examples, the transgenic P. pastoris comprises at most 17 copies of the protein ORF. In some examples, the transgenic P. pastoris comprises at most 16 copies of the protein ORF. In some examples, the transgenic P. pastoris comprises at most 15 copies of the protein ORF. In some examples, the transgenic P. pastoris comprises at most 14 copies of the protein ORF. In some examples, the transgenic P. pastoris comprises at most 13 copies of the protein ORF. In some examples, the transgenic P. pastoris comprises at most 12 copies of the protein ORF. In some examples, the transgenic P. pastoris comprises at most 11 copies of the protein ORF. In some examples, the transgenic P. pastoris comprises at most 10 copies of the protein ORF. According to some embodiments, the P. pastoris is BG11 P. pastori.

The term “at most” when used in conjunction with a value has the meaning of the value or less, but greater than zero.

In some examples, the transgenic P. pastoris comprises between about 10 copies and about 20 copies of the protein ORF; at times, between about 7 copies and about 15 copies of the protein ORF; at times, between 12 copies and about 18 copies of the protein ORF; at times, between about 10 copies and 15 copies of the protein ORF; at times, between about 13 copies and 15 copies of the protein ORF. According to some embodiment, the transgenic P. pastoris comprises from 13 to 15 copies of the protein ORF. According to some embodiment, the transgenic P. pastoris comprises about 14 copies of the protein ORF. According to some embodiments, the P. pastoris is BG11 P. pastori.

According to any one of the above embodiments, the ORF is operably linked to a promoter and a terminator. According to some embodiments, the promoter is AOX1 promoter and the terminator is AOX1 transcription terminator. Thus, according to any one of the above embodiments, each ORF is operably linked to AOX1 promoter and AOX1 terminator. The terms “operably linked”, “operatively linked”, “operably encodes”, and “operably associated” are used herein interchangeably and refer to the functional linkage between a promoter (or terminator) and nucleic acid sequence, wherein the promoter initiates transcription of RNA corresponding to the DNA sequence. A heterologous DNA sequence is “operatively associated” with the promoter in a cell when RNA polymerase which binds the promoter sequence transcribes the coding sequence into mRNA which then in turn is translated into the protein encoded by the coding sequence.

In some examples, the transgenic P. pastoris is a Mut^{S} comprising or consisting of SEQ ID NO:3 in between the AOX1 promoter and the AOX1 transcription terminator. According to some embodiments, the present invention provides P. pastoris BG11 comprising or consisting of SEQ ID NO:3 in between the AOX1 promoter and the AOX1 transcription terminator.

In some examples, the transgenic P. pastoris is a Mut^{S} comprising or consisting of a nucleic acid encoding the amino acid SEQ ID NO:4 in between the AOX1 promoter and the AOX1 transcription terminator.

### Methods of Preparation of Transgenic BG11

The transgenic P. pastoris of the present invention can be obtained by any method known in the art.

In some examples, the P. pastoris such as BG11 is one transformed with a vector comprising a nucleic acid encoding for the beta-lactoglobulin (BLG) protein. According to some embodiments, the vector is a plasmid. According to some embodiments, the nucleic acid encoding the beta-lactoglobulin (rBLG) protein has a nucleic acid SEQ ID NO: 3 or a conservative analog thereof.

The plasmid utilized for the production of the transgenic P. pastoris disclosed herein can be of any type that can be utilized for protein expression in P. pastoris. According to one embodiment, the plasmid is pJAG. According to another embodiment, the plasmid is or pJAN. According to some embodiments, the P. pastoris is transformed with pJAG and pJAN plasmids.

In some examples, the vector, such as a plasmid, comprises the AOX1 promoter. In some examples, the vector, such as a plasmid, comprises the AOX1 transcription terminator. In some examples, the vector, such as a plasmid, comprises both the AOX1 promoter and the AOX1 transcription terminator.

In some examples, the plasmid comprises an ORF of rBLG protein between the AOX1 promoter and the AOX1 transcription terminator.

In some examples, the plasmid comprises, between the sequences of AOX1 promoter and the AOX1 transcription terminator, a DNA encoding the rBLG protein of the present invention. According to some embodiments, the DNA encoding the rBLG has the nucleic acid sequence SEQ ID NO:3 or a functional homolog thereof. According to some embodiments, the DNA has a nucleic acid sequence having at least 90% sequence identity with SEQ ID NO:3, when optimally aligned therewith. According to some embodiments, the functional homolog has a nucleic acid sequence having at least 90% sequence identity with SEQ ID NO:3, when optimally aligned therewith. According to any one of the above embodiments, the rBLG has the amino acid sequence SEQ ID NO: 4.

In some examples, the plasmid comprises a nucleic acid sequence (AOX1 promoter)-(SEQ ID NO:3)-(AOX1 transcription terminator). In some examples, the plasmid comprises a nucleic acid sequence (AOX1 promoter)-(a conservative homolog of SEQ ID NO:3)-(AOX1 transcription terminator).

According to some embodiments, the method comprises transfecting P. pastoris is BG11 P. pastoris with pJAG and pJAN plasmids, each comprising a nucleic acid (AOX1 promoter)-(SEQ ID NO:3)-(AOX1 transcription terminator). According to some embodiments, the method comprises transfecting P. pastoris is BG11 P. pastoris with pJAG and pJAN plasmids, each comprising a nucleic acid comprising the sequences, from 5′ to 3′ SEQ ID NO:1-SEQ ID NO:3-SEQ ID NO:2. According to some embodiments, the resulting BG11 P. pastoris comprises from 12 to 16, from 13 to 15 or about 14 copies of SEQ ID NO:3.

Methods of Preparation of rBLG Isolate

As already noted above, the plasmid is configured to introduce one or a plurality of copies of the DNA encoding the rBLG protein into the transgenic microorganism's genome, e.g., P. pastoris genome. Such transgenic P. pastoris, e.g., BG11 is then utilized in methods for high yield production of rBLG protein. More specifically, the present invention provides a method of preparation of rBLG isolate using BG11 P. pastoris comprising a plurality of copies of the nucleic acid sequence encoding the rBLG protein, e.g., from 7 to 20 copies. Thus, in accordance with another aspect, the present disclosure provides a method for producing an rBLG isolate comprising a rBLG protein. According to some embodiments, the method comprises at least the steps of:
providing a transgenic P. pastoris, e.g., BG11, comprising a plurality of copies of nucleic acid, such as DNA, encoding rBLG protein;
culturing the transgenic P. pastoris under conditions suitable for expressing the rBLG protein and obtaining a rBLG supernatant; and
purifying the rBLG supernatant to obtain a purified supernatant comprising the rBLG protein.

All terms, embodiments and definitions disclosed in any one of the above aspects apply and are encompassed herein as well.

In some examples, the conditions suitable for expressing the rBLG protein include fermentation. In some examples, the method comprises at least one step of fermentation. In some examples, the method comprises two or more steps of fermentation. In some examples, the method comprises two or more steps of fermentation under the same or different conditions. In some examples, the method comprises at least one fermentation step at a temperature of between 25 and 35° C. In some examples, the method comprises at least one fermentation step at a pH of between 5.5 and 6.5. According to some embodiments, the method comprises at least one fermentation step at a temperature of between 25 and 35° C. at a pH of between 5.5 and 6.5.

In some examples, the method comprises at least one fermentation step comprising mixing or agitation at a velocity of between 300 rpm and 600 rpm (the velocity may increase with the time to maintain dissolved oxygen (DO) levels in the medium to compensate for the increasing Pichia numbers in the medium).

Culturing continues until maximal amounts of the protein are accumulated before the culture gains cell-debris impurities. In some embodiments, the expression step, e.g., fermentation steps, lasts for from 50 to 90 or from 60 to 80 hours upon initiation of the expression. According to some embodiments, the promoter used in AOX1 promoter and the expression is initiated by addition of methanol. Once culturing has been completed, the protein is purified.

In some examples, the purification involves the separation of supernatant from the P. pastoris material (including debris therefrom). The purification may involve centrifugation.

In some examples, purification involves the filtration of the supernatant. In some examples, the filtration involves the use of a mesh of about 0.2 microns to remove solids, and/or the use of a membrane of about 3-5 kDa to remove solutes. The purified rBLG supernatant/filtrate comprising the rBLG protein is then collected.

According to some embodiments, the rBLG supernatant/filtrate is further concentrated. Any method known in the art may be used.

In some examples, the purified rBLG supernatant is subjected to additional processing steps to obtain a rBLG protein isolate.

In some examples, the purified rBLG supernatant is subjected to dialysis against water. Without being bound by any theory or mechanism, it is believed that the dialysis against water removes salts, small solutes, volatiles, odors, and microorganism-related aftertaste.

In some examples, the purified rBLG, either supernatant, filtrate or dialyzed rBLG is subjected to concentration or drying. According to some embodiments, the drying is spray drying. According to some embodiments, the drying is freeze drying. Any known methods of concentration and/or drying may be used.

In some embodiment, the obtained rBLG protein isolate comprises at least 60 wt % rBLG protein. In some embodiments, the obtained rBLG isolate comprises at least 65 wt % rBLG protein. In some embodiments, the obtained rBLG isolate comprises at least 70 wt % rBLG protein. In some embodiments, the obtained rBLG isolate comprises at least 75 wt % rBLG protein. It has been found that following drying and/or concentration, the resulting rBLG protein isolate contains at least 80 wt % rBLG protein, at least 82 wt % of rBLG protein, and preferably more than 83 wt % rBLG protein. It has been found that following drying or concentration, the resulting rBLG protein isolate contains at least 90 wt % rBLG protein, and preferably more than 90 wt % rBLG protein. In some examples, the resulting rBLG protein isolate contains at least 91 wt % rBLG protein. In some examples, the resulting rBLG protein isolate contains at least 92 wt % rBLG protein. In some examples, the resulting rBLG protein isolate contains at least 93 wt % rBLG protein. In some examples, the resulting rBLG protein isolate contains at least 94 wt % rBLG protein. In some examples, the resulting rBLG protein isolate contains at least 95 wt % rBLG protein. In some examples, the resulting rBLG protein isolate contains at least 96 wt % rBLG protein. In some examples, the resulting rBLG protein isolate contains at least 97 wt % rBLG protein. In some examples, the resulting rBLG protein isolate contains at least 98 wt % rBLG protein. In some examples, the resulting rBLG protein isolate contains at least 99 wt % rBLG protein. In some embodiments, the rBLG isolate comprises from 90 to 99 wt % of rBLG protein. According to other embodiments, the rBLG isolate comprises from 91 to 98 wt %, from 92 to 97 wt %, from 93 to 96 wt %, from 93 to 99 wt %, or from 94 to 98 wt % of rBLG protein. According to other embodiments, the rBLG isolate comprises from 80 to 100 wt %, from 85 to 95 wt %, or from 80 to 95 wt % of rBLG protein. In some embodiments, the obtained rBLG isolate comprises at least 60 wt % rBLG protein out of the solid content of the isolate. In some embodiments, the obtained rBLG isolate comprises at least 65 wt % rBLG protein out of the solid content of the isolate. In some embodiments, the obtained rBLG isolate comprises at least 70 wt % rBLG protein out of the solid content of the isolate. In some embodiments, the obtained rBLG isolate comprises at least 75 wt % rBLG protein out of the solid content of the isolate. In some embodiments, the obtained rBLG isolate comprises at least 80 wt % obtained rBLG protein out of the solid content of the isolate. In some embodiments, the obtained rBLG isolate comprises at least 85 wt % rBLG protein out of the solid content of the isolate. In some embodiments, the obtained rBLG isolate comprises at least 90 wt % rBLG protein out of the solid content of the isolate.

The level of purity of the protein isolate can be determined by any technique known in the art. In some examples, the rBLG composition and thus level of purity is determined using SEC or reverse-phase high-performance liquid chromatography (RP-HPLC).

In some embodiments, the protein isolate is identified by a single peak at the retention time of about 5.9 min when performing SEC HPLC utilizing PROTEEMA HPLC Column 150×8 mm PSS+50×8 mm guard and using the following eluent” phosphate buffer 20 m pH7 100 mM NaCl.

According to any one of the above embodiments, the rBLG protein isolate comprises at least a trace amount of impurities. The present disclosure also provides a rBLG protein isolate comprising at least 60 or at least 70 wt %, at least 80 wt %, at least 85 wt % or at least 90 wt % of the rBLG protein and optionally at least a trace amount of impurities. The present disclosure also provides a rBLG protein isolate comprising at least 90 wt % the rBLG protein and at least a trace amount of substances/material associated with P. pastoris (these trace amounts may be considered or form part of the impurities). In the context of the present disclosure when referring to trace amounts of material associated with P. pastoris it is to be understood as any by-product of expression of the rBLG DNA in a transgenic P. pastoris as disclosed herein. A by-product in the context of the present disclosure can mean any one or combination of rBLG protein fragments, P. pastoris metabolites, P. pastoris nucleic acids and P. pastoris debris. In some embodiments, the impurities may be rBLG fragments.

The present disclosure also provides a rBLG protein isolate obtained or obtainable by the method disclosed herein. In some examples, the present disclosure provides a rBLG protein isolate obtained by the method disclosed herein. In some other examples, the present disclosure provides a rBLG protein isolate obtainable by the method disclosed herein. The present disclosure also provides a rBLG protein obtained or obtainable from the transgenic P. pastoris disclosed herein.

rBLG Isolate

According to another aspect, the present disclosure also provides an isolate of a recombinant BLG protein. The produced protein can be in the form of an essentially pure protein isolate. All terms, embodiments and definitions disclosed in any one of the above aspects apply and are encompassed herein as well.

As used herein, the terms “purify,” “purified,” and “purifying” or “isolate”, “isolated,” or “isolating” or “enrich,” “enriched” or “enriching” are used interchangeably and refer to the state of a material or compound that has undergone one or more processes of purification, e.g., a selection, enrichment and/or removal or reduction of residual biological products.

The term “rBLG isolate” as used herein refers to the isolated rBLG comprising at least 60 wt % of rBLG. In the context of the present disclosure, when referring to an essentially pure protein isolate it is to be understood as referring to a composition comprising at least 90 wt % rBLG protein.

In some embodiments, the rBLG isolate comprises at least 65 wt % rBLG protein. In some embodiments, the rBLG isolate comprises at least 70 wt % rBLG protein. In some embodiments, the rBLG isolate comprises at least 75 wt % rBLG protein. In some embodiments, the rBLG isolate comprises at least 80 wt % rBLG protein. In some embodiments, the rBLG isolate comprises at least 83 wt % rBLG protein. In some embodiments, the rBLG isolate comprises at least 84 wt % rBLG protein. In some examples, the essentially pure protein isolate comprises at least 95 wt % rBLG protein. In some examples, the essentially pure protein isolate comprises at least 98 wt % rBLG protein. In some examples, the essentially pure protein isolate comprises at least 99 wt % rBLG protein. In some embodiments, the rBLG isolate comprises from 80 to 99 wt % rBLG protein. In some embodiments, the rBLG isolate comprises from 80 to 95 wt % rBLG protein. In some embodiments, the rBLG isolate comprises from 80 to 90 wt % rBLG protein. In some embodiments, the rBLG isolate comprises from 82 to 87 wt % rBLG protein. According to some embodiments, the rBLG has the amino acid sequence SEQ ID NO: 4. According to some embodiments, the protein is an analogue of BLG having at least 85%, at least 90% or at least 95% sequence identity to SEQ ID NO: 4.

In some embodiments, the rBLG isolate comprises at least 55 wt %, or at least 60 wt % rBLG protein out of the solid content of the isolate. In some embodiments, the rBLG isolate comprises at least 65 wt % rBLG protein out of the solid content of the isolate. In some embodiments, the rBLG isolate comprises at least 70 wt % rBLG protein out of the solid content of the isolate. In some embodiments, the rBLG isolate comprises at least 75 wt % rBLG protein out of the solid content of the isolate. In some embodiments, the rBLG isolate comprises at least 80 wt % rBLG protein out of the solid content of the isolate. In some embodiments, the rBLG isolate comprises at least 85 wt % rBLG protein out of the solid content of the isolate. In some embodiments, the rBLG isolate comprises at least 90 wt % rBLG protein out of the solid content of the isolate.

When referring to the rBLG protein in the context of the protein isolate it is to be understood to encompass a protein encoded by the DNA sequence of SEQ ID NO:3 or a functional homolog thereof.

When referring to a functional homolog it is to be understood to encompass a nucleic acid sequence that is capable of encoding for a rBLG protein. According to some embodiments, the functional homolog encodes for a rBLG protein having amino acid SEQ ID NO: 4.

In some examples, the rBLG protein isolate comprises rBLG encoded by a DNA sequence having at least 90% sequence identity with SEQ ID NO:3, when optimally aligned with the SEQ ID NO:3, and encodes for a rBLG protein having the amino acid sequence SEQ ID NO:4.

In some examples, when referring to the rBLG protein isolate, it is to be understood to encompass a protein having the amino acid sequence denoted as SEQ ID NO:4.

In some examples, when referring to the rBLG protein isolate it is to be understood to encompass a protein having at least 90% sequence identity with SEQ ID NO:4, when optimally aligned therewith.

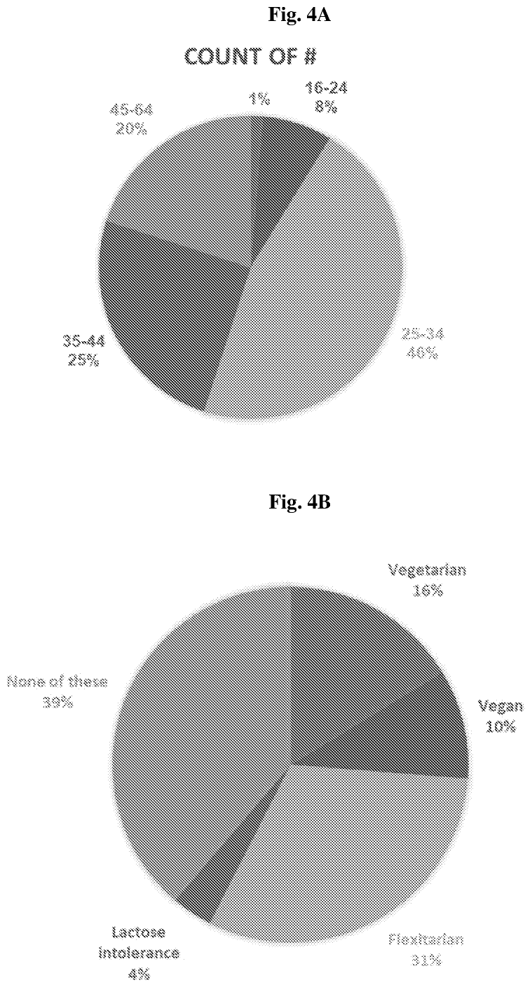

According to some embodiments, the rBLG of the present invention is characterized by at least one peak between elution time of 10.6 to 11 minutes using BioBasic-18, 150×4.6 mm, 5 μm; Column and Mobile phase A: 0.05% of TFA in MQW, Mobile phase C: 0.05% of TFA in Acetonitrile. According to some embodiments, the rBLG is characterized by a major peak around elution time of 10.86 min. According to some embodiments, the rBLG is characterized by a chromatogram as in FIG. 3A.

According to some embodiments, the rBLG of the present invention is characterized by at least one peak between elution time of 5 to 6.5 min, when the isolate is eluted through PROTEEMA GPLC Column 150×8 mm PSS+50×8 mm guard column using 20 mM Phosphate buffer, pH 7, 100 mM NaCl+0.05% Sodium Azide as a running phase exploiting HPLC. According to some embodiments, the rBLG is characterized by a major peak around elution time of 5.83. According to some embodiments, the rBLG is characterized by a chromatogram as in FIG. 3B.

### General Terms

The following terms are relevant to the following aspects and embodiments, and are contemplated, embedded and are part of each one of the relevant embodiments in which these terms are mentioned or used.

As used herein, the forms “a”, “an” and “the” include singular as well as plural references unless the context clearly dictates otherwise. For example, the term “a protein” includes one or more copies of the recited protein.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

Further, all numerical values, e.g., when referring to the amounts or ranges include approximations which are varied (+) or (−) by up to 10%, at times by up to 5% of from the stated values. It is to be understood, even if not always explicitly stated that all numerical designations are preceded by the term “about”. As used herein, the term “about”, when referring to a measurable value such as an amount, a temporal duration, and the like, is meant to encompass variations of +/−10%, or +/−5%, +/−1%, or even +/−0.1% from the specified value.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “composition” when referring to a composition comprising BLG is intended to encompass a product comprising any BLG, e.g., the rBLG of the present invention, and optionally a carrier and/or excipient(s) e.g., sugars. According to some embodiments, the composition is an edible composition comprising only edible ingredients. The term “edible ingredient” as used herein means an organic material that can be consumed, digested, or passed through the digestive system of an animal or human without any toxic effect. These edible ingredient materials can exist as either a solid or liquid at room temperature.

The terms “pre-mix”, “pre-mixture”, “pre-mix composition” and “pre-mix formulation” are used herein interchangeably and refer to mixtures of BLG and other ingredients, pre-mixed in order to facilitate and case the formation of final edible products comprising BLG. It should be understood that the identity of each of the ingredients, and the relative amount of each of the ingredients, is determined and tailor-maid in thought of step (i) the type and desired attributes and characteristics of the final edible product to be produced from the pre-mix, and (ii) the case of use of transforming the pre-mix into the final edible product. The pre-mix composition of the present invention is substantially devoid of coagulation minerals or salts thereof. According to some embodiments, the pre-mix composition comprises a chelating agent. According to some embodiments, the pre-mix is a dry composition. According to some embodiments, the pre-mix is a powder. Another benefit of pre-mixes according to the present invention is the prevention of the necessity to transport large quantities of water and fats from the production facility of the BLG protein to the locations in which it is used to formulate consumer products. The pre-mixture dedicated for use in the preparation of a cream cheese analogue is defined as a “cream cheese premix composition”, and so on.

The terms “cream cheese”, “yogurt”, “cheese”, “ice cream”, and “Ricotta” are well known in the art. The term “cream cheese” is well known in the art, and defined e.g., in Brghenti M. et al. 2008, “Characterization of the Rheological, Textural, and Sensory Properties of Samples of Commercial US Cream Cheese with Different Fat Contents” J. Dairy Sci. 91:4501-4517.

Cream Cheese analogues of the present invention have at least one property of corresponding Cream Cheese dairy products selected from the group consisting of appearance, glossiness, consistency, structure, thickness, taste flavour and penetration strength. According to some embodiments, the cream cheese analogues of the present invention have 2, 3, 4, 5, 6, 7 or 8 properties of corresponding Cream Cheese dairy products. The desired properties are as follows: Appearance: white milky colour; Glossiness: matt interior with a shiny surface; Consistency: slightly adhesive, covering the mouth but melt quickly; Structure: dense without air formation; Thickness: able to build up on a spoon; Flavour: milky, sour, cultured, acetaldehyde, alcohol, buttery (not from proteins); Taste: sour, sweet, salty; and Penetration strength: 80-100[g].

Yogurt analogues of the present invention have at least one property of corresponding Yogurt dairy products selected from the group consisting of appearance, glossiness, consistency, structure, thickness, and taste flavour. According to some embodiments, the yogurt analogues of the present invention have 2, 3, 4, 5, 6, or 7 properties of corresponding Yogurt dairy products. The desired properties are as follows: Appearance: white milky colour; Glossiness: matt interior with a shiny surface; Consistency: slightly adhesive, covering the mouth but melt quickly; Structure: dense without air formation; Thickness: able to build up on a spoon; Flavour: milky, sour, cultured, acetaldehyde, alcohol, buttery (not from proteins); Taste: sour, sweet, and salty.

Ricotta analogues of the present invention have at least one property of corresponding Ricotta dairy products selected from the group consisting of appearance, glossiness, consistency, structure, thickness, taste, and flavour. According to some embodiments, the Ricotta cheese analogues of the present invention have 2, 3, 4, 5, 6, or 7 properties of corresponding Ricotta cheese dairy products. The desired properties are as follows: Appearance: white milky colour; Glossiness: matt interior with a shiny surface; Consistency: slightly adhesive, covering the mouth but melt quickly; Structure: dense without air formation; Thickness: able to build up on a spoon; Flavour: mild milky, buttery (not from protein); and Taste: Sweet, Salty, metallic.

Ice cream analogues of the present invention have at least one property of corresponding Ice cream dairy products selected from the group consisting of appearance, glossiness, consistency, structure, thickness, taste, flavour, and overrun. According to some embodiments, the Ice cream analogues of the present invention have 2, 3, 4, 5, 6, 7 or 8 properties of corresponding Ice cream dairy products. The desired properties are as follows: Appearance: white milky colour; Glossiness: shiny surface and interior; Consistency: slightly adhesive, covering the mouth but melt quickly; Structure: Airy but stable at freezing temperature; Thickness: able to build up on a spoon; Flavour: Milky, buttery, and vanilla (not from protein); Taste: Sweet; Overrun: 30-40%.

As used herein, the terms “edible product” and “food product” refer to a product that is suited for human or animal, preferably human, consumption.

As used herein, the terms “dairy substitute”, “dairy alternative”. “dairy analogue” and “dairy analogue food product” are used herein interchangeably and refer to any consumable/edible product or foodstuff, which is not made from or derived from animals' milk. In some embodiments, the terms also refer to such products that comprise combinations of ingredients that are originated from animals' milk and those that are not. Such products may replace animal-based products in one's diet by attempting to mimic or equal the rheologic and/or organoleptic/physicochemical properties of the traditional animal-milk-based products.

The term “non-dairy” as contemplated by the present invention is intended to include all types of products that contain no milk that comes from cows, goats or other mammals and are devoid of ingredients derived directly from the milk.

In the context of the present disclosure, when referring to “non-animal” and “animal-free” products it is to be understood as encompassing a product that is entirely free of animal-derived components, such as BLG or other milk proteins. All proteins of such a product are recombinantly-produced.

The terms “coagulation salt”, “coagulation mineral” and “coagulation mineral salt” may be herein interchangeably and refer to a mineral, e.g., in the form of soluble salt, or ions thereof that initiates protein coagulation, as known in the art. It is known that soluble salt upon dissolution disintegrates to ions forming it. Thus, according to some embodiments, the term salt refers also to the dissolved salt. Upon dissolution, coagulation mineral salt provides cations that initiate coagulation. According to some embodiments, the coagulation mineral comprises one or more salts of a mineral selected from the group consisting of calcium, magnesium, phosphorus, potassium, selenium, and zinc. In some examples, the coagulation mineral is calcium or magnesium. In some examples, the coagulation mineral salt is a calcium salt or a magnesium salt. In some examples, the coagulation mineral salt is selected from the group consisting of calcium chloride, magnesium chloride, and calcium lactate. According to some embodiments, the coagulation mineral salt is calcium chloride.

The term “coagulation” or “coagulated” has the meaning of a solidification process in which proteins (and optionally lipids) are made to stick together as a discontinuous phase, thereby becoming separated from their original liquid continuous phase. Typically, coagulation of BLG refers to a partial unfolding of the protein without loss of secondary structures, whereas aggregation of BLG refers to full or almost full denaturation, loss of secondary structures, and, optionally, formation of 100-500 micron fibers.

The terms “substantially devoid”, “essentially devoid”, “devoid”, “does not include” and “does not comprise” may be used interchangeably and refer to a composition or product that does not include, contain, or comprise a particular component, e.g., the composition comprises less than 0.01 wt %, or less than 0.001 wt % of the component. In some embodiments, the term “devoid” contemplates a composition comprising traces of the devoid component such as traces of a component used in the purification process.

The term “substantially devoid of coagulation minerals” has the meaning that the composition or product comprises coagulation minerals at such levels that are insufficient to cause coagulation of the milk proteins in the composition. The term “substantially devoid of coagulation minerals” and “comprising sub-critical concentration of coagulation minerals” may be used interchangeably. The term “sub-critical” as used herein refers to the amount of coagulation minerals that do not cause coagulation. In some embodiments, the amount is from 0 wt % to 0.001 wt %.

The terms “pasteurizing”, “pasteurization” and “pasteurization” are well-known in the art, and generally refer to a process in which foods (such as milk) are treated with heat to eliminate pathogens, thereby extending shelf life. In some embodiments, pasteurization is effected at a temperature of from 65 to 140° C. or from 80 to 95° C. for a period of from 15 seconds to 10 minutes or from 1 to 10 minutes. According to the principles of the present invention, any heating temperature and any heating duration found to pasteurize the compositions and products of the present invention are included.

The term “smoothing” is used herein as well-known in the art. According to some embodiments, the smoothing is effected by homogenization. According to other embodiments, the smoothing is effected by extrusion, e.g., by injecting the material through a fine needle.

The terms “homogenized” and “homogenization” refer to the process or to the product that passed the process of homogenization. The homogenization may be effected by any known method and device. According to some embodiments, the homogenization is performed in 1, 2, 3 or 4 stages. According to some embodiments, the homogenization is effected at from about 50 to about 400 bar. According to the principles of the present invention, any homogenization stage and any homogenization pressure found to homogenize the compositions and products of the present invention are included.

The terms “lipid”, “fat” and “oil” include any edible lipid. According to some embodiments, the lipid is a non-animal lipid. According to some embodiment, the lipid is plant-derived lipid. According to some embodiments, the edible lipids comprise an oil. According to some embodiment, the oil is selected from the group consisting of shea oil, sunflower oil, coconut oil, rapeseed oil, nut oil, palm oil, kernel oil, olive oil, soya oil, cotton oil, and cocoa butter (Theobroma oil).

The term “sugar” refers to any edible sugar, carbohydrate, or sugar substitute. According to some embodiments, the sugar is a non-animal sugar. According to some embodiment, the sugar is plant-derived sugar. According to some embodiments, the sugar is selected from the group consisting of a monosaccharide, disaccharide, and polysaccharide. According to some embodiments, the sugar is selected from the group consisting of glucose, fructose, mannose, xylose, arabinose, sucrose, dextrose, maltose, and galactose.

The term “total milk-protein content” refers to the content of all milk proteins in the composition. In the context of the present disclosure, when referring to “milk protein” it is to be understood as meaning proteins present in milk, yet, not necessarily isolated from milk. In certain embodiments, the term “milk protein” refers to BLG, ALA, αS1 casein, αS2 casein, β casein, and κ casein.

The term “stabilizer” as used herein refers to an additive to food which helps to preserve its structure. Non-limiting examples of stabilizers are functional enzymatically treated potato starch such as Etenia 457, starch, Lcoust bean gum, pectin, Carrageenan, and any combination thereof.

The terms “flavoring salt” or “flavoring mineral” are used herein interchangeably and refer to any salt added to a composition such as a food product in order to change its flavor into a saltier taste. In some embodiments, the flavoring salt is a sodium or a potassium salt or an iodine salt. In some examples, the flavoring salt is selected from the group consisting of sodium chloride, potassium chloride, and potassium lactate; yet, necessarily excludes salts of coagulation minerals such as calcium salts and/or magnesium salts. In the context of the present disclosure, when referring to flavoring salts it is to be understood as any salt that provides a salty flavor, yet does not cause coagulation of the starting protein composition

The term “chelating agent”, “chelating salt”, “chelator”, “coagulation mineral chelator” and “chelating mineral” are used herein interchangeably and refer to agents capable of chelating cations, such as divalent ions. In some embodiments, chelating agent chelates divalent ions. In some embodiments, the chelating agent is added to chelate divalent cation(s) to prevent early or spontaneous coagulation. In some examples, the “chelating salt” or “chelating mineral” is a sodium salt. In some examples, a “chelating salt” or a “chelating mineral” is selected from the group consisting of sodium citrate, trisodium citrate, sodium phosphate, and sodium orthophosphate. In some examples, the salt is in its soluble form. In some other examples, the salt is in dry form.

The term “acidification” refers to the process of reducing the pH of the composition. According to some embodiments, acidification may be effected by any known method. Acidification can be carried out using Lactic Acid Bacteria (LAB) or by using a whey culture. The LAB bacteria can be a mixed-strain or defined-strain cultures. In some examples, the bacteria culture is mesophilic. In some other examples, the bacteria culture is thermophilic. Acidification may be carried out by adding an acid.

The term “effected” is used as well known in the art and has the meaning of made, performed, executed etc.

### Compositions Comprising BLG

According to another aspect, the present invention provides a composition comprising a milk-isolated BLG, a rBLG protein, a rBLG protein of the present invention, or the rBLG isolate of the present invention.

According to some embodiments, the present invention provides an edible product comprising BLG, e.g., the rBLG of the present invention. According to some embodiments, the present invention provides a precursor/pre-mix comprising BLG, the pre-mix is used for the preparation of the edible products of the present invention. The edible compositions or products of the present invention are substitutes or alternatives of traditional dairy products.

According to some embodiments, the present invention provides a dairy analogue food product, comprising the rBLG protein according to any one of the above aspects and embodiments. All terms, embodiments and definitions defined in any one of the above aspects apply and are encompassed herein as well.

According to the principles of the present invention, any recombinant BLG including the recombinant BLG of the present invention is not considered a milk-derived or an animal-derived component.

According to some embodiments, the present invention provides an animal-free dairy analogue food product or a precursor/pre-mix thereof used for the preparation of the food product comprising rBLG.

In other examples, the dairy analogue food product of the present invention is not “animal-free”. Such products may be denoted as “non-animal-free” or “dairy-derived” products. In the context of the present disclosure, when referring to non-animal-free food products it is to be understood as encompassing (i) a product that is free of animal derived proteins but includes animal derived components (such as fat or lactose); or (ii) a product that comprises milk proteins, isolated from animal milk.

According to any one of the above embodiments of the present invention, the dairy analogue food product comprising rBLG is an analogue of cheese. According to some embodiments, the dairy-analogue food product is an analogue of cream cheese. According to some embodiments, the dairy-analogue food product is an analogue of yogurt. According to some embodiments, the dairy-analogue food product is an analogue of ice-cream.

According to some embodiments, the dairy-analogue food product is an animal-free dairy-analogue food product selected from the group consisting of cheese, cream cheese, yogurt, and ice cream. According to some embodiments, the dairy analogue food product is a non-animal-free dairy-analogue of a dairy product selected from the group consisting of cheese, cream cheese, yogurt, and ice cream.

### Methods for Preparing Dairy Analogue Food Products

According to another aspect, the present invention provides a method of preparing a dairy analogue food product comprising a coagulated beta-lactoglobulin B (BLG), the method comprising:
(i) pasteurizing a composition comprising a BLG protein, wherein the composition is substantially devoid of coagulation minerals;
(ii) optionally, cooling down the composition of step (i); and
(iii) adding a coagulation mineral to the composition of step (ii), if present, or to the composition of step (i), if step (ii) is absent, thereby coagulating the BLG protein,
wherein BLG constitutes at least 51 wt % of the total milk-protein content of the dairy analogue food product, and wherein the dairy analogue food product comprises at least about 0.75 wt % BLG.

It should be understood that the method above may be preceded and/or followed by several steps relating to general steps of preparing traditional dairy food products, known to any person of average skill in the art.

All terms, embodiments and definitions defined in any one of the above aspects apply and are encompassed herein as well.

In some examples, the coagulation mineral is calcium or magnesium. In some examples, the coagulation mineral salt is selected from the group consisting of calcium chloride, magnesium chloride, and calcium lactate. According to some embodiments, the coagulation mineral salt is calcium chloride.

According to some embodiments, the resulted dairy analogue food product comprises at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 6 wt %, or at least 6.5 wt % of BLG.

According to some embodiments, the resulted dairy analogue food product comprises from about 0.75 to about 10 wt % of BLG. According to some embodiments, the resulted dairy analogue food product comprises from about 0.75 to about 8 wt % of BLG. According to some embodiments, the resulted dairy analogue food product comprises from about 1 to about 8 wt % of BLG. According to some embodiments, the resulted dairy analogue food product comprises from about 1.5 to about 7.5 wt % of BLG. According to some embodiments, the resulted dairy analogue food product comprises from about 2 to about 6 wt % of BLG. According to some embodiments, the resulted dairy analogue food product comprises from about 2 to about 8 wt % of BLG. According to some embodiments, the resulted dairy analogue food product comprises from about 3 to about 6 wt % of BLG. According to some embodiments, the resulted dairy analogue food product comprises from about 3 to about 8 wt % of BLG.

According to some embodiments, the resulted dairy analogue food product comprises up to 0.35 wt % of a coagulation mineral salt. According to some embodiments, the resulted dairy analogue food product comprises from about 0.0015 to about 0.35 wt % of a coagulation mineral salt(s). According to some embodiments, the resulted dairy analogue food product comprises from about 0.002 to about 0.30 wt % of a coagulation mineral salt(s). According to some embodiments, the resulted dairy analogue food product comprises from about 0.005 to about 0.25 wt % of a coagulation mineral salt(s). According to some embodiments, the resulted dairy analogue food product comprises from about 0.01 to about 0.20 wt % of a coagulation mineral salt(s). According to some embodiments, the resulted dairy analogue food product comprises from about 0.05 to about 0.2 wt % of a coagulation mineral salt(s). According to some embodiments, the resulted dairy analogue food product comprises from about 0.1 to about 0.25 wt % of a coagulation mineral salt(s). According to some embodiments, the coagulation mineral salt is calcium chloride.

According to some embodiments, the resulted dairy analogue food product comprises from about 0.75 to about 10 wt % of BLG and from about 0.005 to about 0.25 wt % of a coagulation mineral salt(s). According to some embodiments, the resulted dairy analogue food product comprises from about 1 to about 8 wt % of BLG and from about 0.01 to about 0.25 wt % of a coagulation mineral salt(s).

As defined hereinabove, the terms “coagulation mineral” and “coagulation mineral salt” may be used interchangeably.

According to one embodiment, the dairy analogue food product comprises from about 1.5 to about 8 wt % BLG, and step (iii) comprises adding from about 0.0015 to about 0.035 wt % of a coagulation mineral. According to another embodiment, the dairy analogue food product comprises from about 1.5 to about 4.5 wt % BLG, and step (iii) comprises adding from about 0.001 about 0.07 wt % of a coagulation mineral. According to yet another embodiment, the dairy analogue food product comprises from about 1.5 to about 3.5 wt % BLG, and step (iii) comprises adding from about 0.001 about 0.1 wt % of a coagulation mineral. According to an alternative embodiment, the dairy analogue food product comprises from about 1 to about 3 wt % BLG, and step (iii) comprises adding from about 0.1 about 0.2 wt % of a coagulation mineral.

According to some embodiments, the dairy analogue food product comprises from about 2 to about 4 wt % BLG, and step (iii) comprises adding about 0.05 wt % of a Coagulation mineral; the dairy analogue food product comprises from about 2 to about 3 wt % BLG, and step (iii) comprises adding about 0.075 wt % of a Coagulation mineral; or the dairy analogue food product comprises from about 1 to about 3 wt % BLG, and step (iii) comprises adding about 0.15 wt % of a Coagulation mineral.

According to any one of the above embodiments, the method further comprises subjecting the dairy analogue food product to acidification to prepare an acidified dairy analogue product.

According to some embodiments, the acidification may be affected by any known method. Acidification can be carried out using Lactic Acid Bacteria (LAB) or by using a whey culture. The LAB bacteria can be a mixed-strain or defined-strain cultures. In some examples, the bacteria culture is mesophilic. In some other examples, the bacteria culture is thermophilic. Acidification may be carried out by adding acid.

According to some embodiments, the acidification is effected until the desired pH is reached.

According to some embodiments, the method further comprises adding a flavoring salt to the dairy analogue food product to produce a salted dairy analogue food product. According to some embodiments, the flavoring salt is sodium chloride.

According to some embodiments, the method further comprises smoothing the dairy analogue food product to produce a smooth dairy analogue food.

Thus, according to some embodiments, the method further comprises subjecting the dairy analogue food product to the following steps: subjecting the dairy analogue food product to a lactic acidification to produce an acidified dairy analogue food product; and/or adding a flavoring salt to the dairy analogue food product to produce a salted dairy analogue food product; and/or smoothing the dairy analogue food product to produce a smooth dairy analogue food.

According to any one of the above embodiments, BLG constitutes at least 53 wt % of the total milk-protein content of the dairy analogue food product. According to some embodiments, BLG constitutes at least 55 wt % of the total milk-protein content of the dairy analogue food product. According to some embodiments, BLG constitutes at least 60 wt %, at least 65 wt %, at least 70 wt %, at least 75 wt %, at least 80 wt %, at least 85 wt %, at least 90 wt %, at least 95 wt %, at least 97 wt %, at least 98% or at least 99% of the total milk-protein content of the dairy analogue food product. According to some embodiments, BLG constitutes from about 51 wt % to about 100 wt %, from about 55 wt % to about 99 wt %, from about 60 wt % to about 98 wt %, from about 65 wt % to about 97 wt %, from about 70 wt % to about 95 wt %, from about 75 wt % to about 93 wt %, from about 80 wt % to about 90 wt %, from about 60 wt % to about 80 wt %, or from about 70% to about 90% of the total milk-protein content of the dairy analogue food product. According to some embodiments, the BLG constitutes from about 55 wt % to about 95 wt %, from 60 wt % to about 90 wt %, from about 65 wt % to about 85 wt %, from about 70% to about 80 wt %, from 70% to about 99 wt %, from 75 wt % to about 95 wt %, from 85% to about 95 wt %, from 90 wt % to about 100 wt % or from 95 wt % to about 99 wt % of the total milk-protein content of the final dairy analogue food product. According to some embodiments, the BLG is the sole milk protein in the final dairy analogue food product.

According to any one of the above embodiments, BLG constitutes at least 51 wt % of the total protein content of the dairy analogue food product. According to any one of the above embodiments, BLG constitutes at least 53 wt % of the total protein content of the dairy analogue food product. According to some embodiments, BLG constitutes at least 55 wt % of the total protein content of the dairy analogue food product. According to some embodiments, BLG constitutes at least 60 wt %, at least 65 wt %, at least 70 wt %, at least 75 wt %, at least 80 wt %, at least 85 wt %, at least 90 wt %, at least 95 wt %, at least 97 wt %, at least 98 wt % or at least 99 wt % of the total protein content of the dairy analogue food product. According to some embodiments, BLG constitutes from about 51 wt % to about 100 wt %, from about 55 wt % to about 99 wt %, from about 60 wt % to about 98 wt %, from about 65 wt % to about 97 wt %, from about 70 wt % to about 95 wt %, from about 75 wt % to about 93 wt %, from about 80 wt % to about 90 wt %, from about 60 wt % to about 80 wt %, or from about 70 wt % to about 90 wt % of the total protein content of the dairy analogue food product. According to some embodiments, the BLG constitutes from about 55 wt % to about 95 wt %, from 60 wt % to about 90 wt %%, from about 65 wt % to about 85%, from about 70 wt % to about 80 wt %, from 70 wt % to about 99 wt %, from 75 wt % to about 95 wt %, from 85 wt % to about 95 wt %, from 90 wt % to about 100 wt % or from 95 wt % to about 99 wt % of the total protein content of the final dairy analogue food product. According to some embodiments, the BLG is the sole protein in the final dairy analogue food product.

According to any one of the above embodiments, the final dairy analogue food product comprises coagulated BLG. According to some embodiments, at least about 40%, at least about 45%, at least about 50%, at least about 55%, at least about 60%, at least about 65%, at least about 70%, at least about 75%, at least about 80%, at least about 85%, at least about 90% or at least about 95% of the BLG is coagulated. According to some embodiments, from about 40% to about 100%, from about 45% to about 99%, from about 50% to about 95%, from about 55% to about 95%, from about 60% to about 94%, from about 65% to about 93%, from about 70% to about 92%, from about 75% to about 90%, from about 80% to about 90%, from about 80% to about 95%, from about 85% to about 95% or from about 85 to about 99% of the BLG is coagulated.

According to another embodiment, the present invention provides a method for preparing a dairy-analogue food product comprising a coagulated beta-lactoglobulin B (BLG), the method comprising:
(i) pasteurizing a composition comprising a BLG protein and substantially devoid of coagulation minerals;
(ii) optionally, cooling down the composition of step (i); and
(iii) adding to the composition of step (i), if step (ii) is absent, or of step (ii) from 0.001 to 0.35 wt % of a coagulation mineral, thereby coagulating the BLG protein to obtain the dairy-substitute food product,
wherein BLG constitutes from 51 to 100 wt % of the total milk-protein content of the dairy-substitute food product, and
wherein the dairy-substitute food product comprises from 1 to 10 wt % of the BLG.

According to some embodiments, the method comprises the step (ii). According to any one of the above embodiments, the composition of step (i) further comprises a sugar. According to some embodiments, the sugar is selected from the group consisting of a monosaccharide, disaccharide, and polysaccharide. According to some embodiments, the sugar is selected from the group consisting of glucose, fructose, mannose, xylose, arabinose, sucrose, dextrose, maltose, and galactose.

According to any one of the above embodiments, the composition of step (i) is a homogenized composition.

The pasteurization may be effected as known in the art e.g., at a temperature of from 65 to 140° C. or from 80 to 95° C. for a period of from 15 seconds to 10 minutes or from 1 to 10 minutes.

According to some embodiments, the dairy-substitute food product comprises from 1 to 10 wt % of BLG, and step (iii) comprises adding from 0.001 to 0.045 wt % of calcium chloride.

According to some embodiments, the dairy-substitute food product comprises from 1 to 4 wt % of BLG, and step (iii) comprises adding from 0.05 to 0.3 wt % of calcium chloride.

According to any one of the above embodiments, the method further comprises adding from 1 to 40 wt % of edible lipids to the homogenized composition prior to pasteurization. Thus, according to some embodiments, the homogenized composition of step (i) further comprises from 1 to 40 wt % of edible lipids. According to some embodiments, the edible lipids comprise non-animal-derived lipids. According to some embodiments, the edible lipids comprise and oil. According to some embodiment, the oil is selected from the group consisting of shea oil, sunflower oil, coconut oil, rapeseed oil, nut oil, palm oil, kernel oil, olive oil, soya oil, cotton oil, and cocoa butter (Theobroma oil). According to some embodiments, prior to adding, a non-liquid lipid is heated until liquidized.

According to some embodiments, the acidification is performed by adding to the dairy-substitute food product lactic acid bacteria and fermenting to a desired pH. According to some embodiments, the fermentation is terminated when the pH reaches the value of at least 3.7 or at pH of from 3.7 to 6.

According to some embodiments, the method further comprises adding a citrate salt. According to some embodiments, the citrate salt is added in a soluble form, in an amount of between about 0.01% and about 0.2%.

According to any one of the above embodiments, the BLG is a rBLG. According to any one of the above embodiments, the BLG is the rBLG of the present invention.

According to some embodiments, the present invention provides a dairy analogue food product prepared according to the method according to any one of the above embodiments. According to some embodiments, the present invention provides a dairy analogue food product obtained or obtainable by the method according to any one of the above embodiments. According to some embodiments, the dairy analogue food product is selected from the group consisting of a cream cheese analogue, a Ricotta cheese analogue, an ice cream analogue, and yogurt analogue. According to some embodiments, the dairy analogue food product has the organoleptic and/or rheologic properties of the dairy food product.

According to another aspect, the present invention provides a method of preparing a dairy analogue food product comprising a coagulated BLG protein, the method comprising:
(i) pasteurizing a composition comprising a BLG protein, wherein the composition is substantially devoid of coagulation minerals;
(ii) optionally, cooling down the composition of step (i); and
(iii) acidifying the composition of step (i), if step (ii) is absent, or of step (ii), thereby coagulating the BLG protein;
wherein BLG constitutes at least 51 wt % of the total milk-protein content of the dairy analogue food product, and
wherein the dairy analogue food product comprises at least about 2 wt % BLG.

According to some embodiments, the method comprises step (ii).

All terms, embodiments and definitions defined in any one of the above aspects apply and are encompassed herein as well.

According to some embodiment, the dairy analogue food product is obtained upon acidification. According to some embodiments, the resulted dairy analogue food product comprises from 1.5 to 8 wt % of BLG. According to some embodiments, the resulted dairy analogue food product comprises from about 1.5 to about 7.5 wt % of BLG. According to some embodiments, the resulted dairy analogue food product comprises from about 2 to about 6 wt % of BLG. According to some embodiments, the resulted dairy analogue food product comprises from about 2 to about 8 wt % of BLG. According to some embodiments, the resulted dairy analogue food product comprises from about 3 to about 6 wt % of BLG. According to some embodiments, the resulted dairy analogue food product comprises from about 3 to about 8 wt % of BLG.

According to some embodiments, the acidification is performed by adding lactic acid bacteria to the dairy analogue food product and fermenting. According to some embodiments, the acidification is effected until the desired pH is reached. According to some embodiments, the fermentation is terminated when the pH reaches the value of at least 3.7 or at pH of from 3.7 to 6.

According to some embodiments, the method further comprises adding a flavoring salt to the dairy analogue food product to produce a salted dairy analogue food product. According to some embodiments, the flavoring salt is sodium chloride.

According to some embodiments, the method further comprises smoothing the dairy analogue food product to produce a smooth dairy analogue food. According to some embodiments, the final dairy analogue food product comprises coagulated BLG. According to some embodiments, at least about 40%, at least about 45%, at least about 50%, at least about 55%, at least about 60%, at least about 65%, at least about 70%, at least about 75%, at least about 80%, at least about 85%, at least about 90% or at least about 95% of the BLG is coagulated. According to some embodiments, from about 40% to about 100%, from about 45% to about 99%, from about 50% to about 95%, from about 55% to about 95%, from about 60% to about 94%, from about 65% to about 93%, from about 70% to about 92%, from about 75% to about 90%, from about 80% to about 90%, from about 80% to about 95%, from about 85% to about 95% or from about 85 to about 99% of the BLG is coagulated. According to any one of the above embodiments, the BLG is rBLG. According to any one of the above embodiments, the BLG is the rBLG of the present invention.

According to any one of the above embodiments, the method further comprises adding from 1 to 40 wt % of edible lipids to the homogenized composition prior to pasteurization. Thus, according to some embodiments, the homogenized composition of step (i) further comprises from 1 to 40 wt % of edible lipids.

According to another embodiment, the present invention provides a method for preparing a dairy-analogue food product comprising a coagulated BLG, the method comprising:
(i) pasteurizing a composition comprising BLG and substantially devoid of coagulation minerals;
(ii) optionally, cooling down the composition of step (i); and
(iii) subjecting the dairy-substitute food product to a lactic acidification to initiate BLG coagulation and to produce an acidified dairy-substitute food; and
(iv) adding flavoring salt to the product obtained in step (i), step (ii) or step (iii) to produce a salted dairy-substitute food; and
(v) optionally, smoothing the salted dairy-substitute food product of step (iv);
wherein BLG constitutes from 51 to 100 wt % of the total milk-protein content of the dairy-substitute food product, and
wherein the dairy-substitute food product comprises from 2 to 10 wt % of BLG.

According to some embodiments, the composition of step (i) further comprises a sugar. According to other embodiments, the composition of step (i) is a homogenized composition.

According to some embodiments, the present invention provides a dairy analogue food product prepared according to the method according to any one of the above embodiments. According to some embodiments, the present invention provides a dairy analogue food product obtained or obtainable by the method according to any one of the above embodiments. According to some embodiments, the dairy analogue food product is selected from the group consisting of a cream cheese analogue, a Ricotta cheese analogue, an ice cream analogue, and a yogurt analogue. According to some embodiments, the dairy analogue food product has the organoleptic and/or rheologic properties of the dairy food product.

The dairy analogues of the present as described in any one of the above and below embodiments, may have a texture score of from 0 to 150. The texture score may be measured according to any method known in the art, e.g., as described in Example 12. According to some embodiments, the texture score is measured using a texture analyser, e.g., T.A micro stable using probe p/6. According to some embodiments, the dairy analogue has a texture score of about 0. According to some embodiments, the dairy analogue has a texture score of from 0 to 10, e.g., about 1, about 2, about 3, about 4, about 5, about 6, about 7, about 8, about 9 or about 10. According to some embodiments, the dairy analogue has a texture score of above 0, above 1, above 2, above 3, above 4, above 5, above 6, above 7, above 8, above 9 or above 10. According to some embodiments, the dairy analogue has a texture score of at least 1, at least 2, at least 3, at least 4, at least 5, at least 6, at least 7, at least 8, at least 9 or at least 10. According to some embodiments, the dairy analogue has a texture score of at least 15, at least 20, at least 25, at least 30, at least 35, at least 40, at least 45, at least 50, at least 55, at least 50, least 65, at least 70, at least 75, at least 80, at least 85, at least 90, at least 95, at least 100, or at least 120. According to some embodiments, the dairy analogue has a texture score of from 10 to 120, from 20 to 110, from 30 to 100, from 40 to 90, from 50 to 80, from 60 to 70, from 15 to 70 or from 20 to 60.

The dairy analogues of the present as described in any one of the above and below embodiments, may have a color parameter L of from about 40 to about 80. The means for measuring color and color parameters are well known in art. The color may be measured by any known method, e.g., by Chromameter as described in Example 12. The term color parameter L refers to the lightness of the color. According to some embodiments, the dairy analogues have a color parameter L of from about 45 to about 75, from about 50 to about 70, from about 55 to about 65.

According to yet another aspect, the present invention provides a method for coagulating BLG, the method comprising:
(i) pasteurizing a composition comprising from 1 to 70 wt % BLG, and optionally a sugar, wherein the composition is substantially devoid of coagulation minerals;
(ii) optionally, cooling down the composition of step (i); and
(iii) adding to the composition of step (i) or step (ii) from 0.001 to 0.35 wt % of a coagulation mineral, thereby coagulating the BLG,
wherein BLG constitutes from 51 to 100 wt % of the total milk-protein content of the dairy-substitute food product.

All terms, embodiments and definitions defined in any one of the above aspects apply and are encompassed herein as well.

### Pre-Mix Compositions

In some examples, the present disclosure provides pre-mixes that may be used in the production of the dairy analogue food products. According to some embodiments, the premixture is a non-dairy product. According to some embodiments, the premixture is a non-animal product. Such premixtures comprise dry BLG, which may comprise rBLG, e.g., the rBLG isolate disclosed herein. Thus, according to some embodiments, the present invention provides a dairy analogue pre-mixture non-dairy and/or animal-free composition comprising BLG. According to some embodiments, the pre-mix composition is a dry composition. According to other embodiments, the pre-mix composition is a powder.

According to other alternative embodiments, the premixtures may comprise BLG isolated from milk. Thus, according to some embodiments, the present invention provides a dairy analogue pre-mixture non-animal-free composition comprising the BLG isolated from milk. Such premixtures may be used in preparation of non-animal-free dairy analogue food products.

The premix compositions of the present invention are substantially devoid of coagulation minerals in case the pre-mix is used for preparation of a dairy analogue food product comprising a coagulated BLG. According to some embodiments, the pre-mix compositions of the present invention comprise sub-critical concentration of coagulation minerals.

According to some embodiments, the pre-mix composition comprises a chelating agent.

In some examples, the present disclosure provides a pre-mixture that may be used in the production of cream-cheese analogue. In some embodiments, the pre-mixture is for the preparation of dairy analogue products cream cheese. Such premixtures comprise BLG or rBLG. Thus, according to some embodiments, the present invention provides an animal-free dairy analogue pre-mixture compositions comprising the rBLG isolate of the present invention.

According to other alternative embodiments, the premixtures may comprise BLG isolated from milk. In some examples, the pre-mix composition comprises or consists essentially of the BLG protein and at least a sweetener.

In some examples, the pre-mix compositions comprise a bulking agent, such as a sweetener. In some embodiments, the bulking agent such as a sweetener, comprises a saccharide-containing molecule. According to some embodiments, the saccharide-containing molecule can be a monosaccharide, a disaccharide, an oligosaccharide, or polysaccharide. In some examples, the saccharide is a monosaccharide selected from the group consisting of sucrose, glucose, dextrose (D-glucose), fructose, mannose, xylose, arabinose, and galactose. In some examples, the saccharide is dextrose. In some examples, the saccharide is not lactose. In some examples, the saccharide is not animal-derived lactose. In some examples, the saccharide is a synthetic or semi-synthetic lactose.

### Cream Cheese Analogue Pre-Mix Compositions

According to another aspect, the present invention provides a cream cheese analogue pre-mix composition, comprising:
(i) from about 35 to about 80 wt % BLG;
(ii) from about 15 to about 45 wt % of a sugar; and
(iii) from about 0.5 to about 45 wt % of a stabilizer;
wherein the pre-mix composition is substantially devoid of coagulation minerals, and
wherein BLG constitutes at least 51 wt % of the total milk-protein content of the pre-mix composition.

According to some embodiments, the cream cheese analogue pre-mix composition further comprises from about 0.8 to about 2 wt % of a coagulation mineral chelator.

All terms, embodiments and definitions disclosed in any one of the above aspects apply and are encompassed herein as well.

According to some embodiments, the cream cheese analogue pre-mix composition comprises (i) from about 35 to about 50 wt % BLG; (ii) from about 15 to about 25 wt % of a sugar, and (iii) from about 25 to about 45 wt % of a stabilizer.

According to some embodiments, the cream cheese analogue pre-mix composition comprises (i) from about 35 to about 50 wt % BLG; (ii) from about 17 to about 25 wt % of a sugar; and (iii) from about 30 to about 45 wt % of a stabilizer.

According to some embodiments, the cream cheese analogue pre-mix composition comprises (i) from about 35 to about 55 wt % BLG; (ii) from about 10 to about 30 wt % of a sugar; and (iii) from about 20 to about 50 wt % of a stabilizer.

According to some embodiments, the cream cheese analogue pre-mix composition comprises (i) from about 30 to about 50 wt % BLG; (ii) from about 10 to about 30 wt % of a sugar; and (iii) from about 30 to about 50 wt % of a stabilizer.

According to some embodiments, the cream cheese analogue pre-mix composition comprises (i) from about 65 to about 85 wt % BLG; (ii) from about 10 to about 30 wt % of a sugar; and (iii) from about 0.5 to about 2 wt % of a stabilizer.

According to some embodiments, the cream cheese analogue pre-mix composition comprises (i) from about 70 to about 80 wt % BLG; (ii) from about 20 to about 25 wt % of a sugar; and (iii) from about 0.5 to about 3 wt % of a stabilizer.

According to some embodiments, the cream cheese analogue pre-mix composition comprises (i) from about 30 to about 55 wt % BLG; (ii) from about 10 to about 30 wt % of a sugar; and (iii) from about 20 to about 40 wt % of a stabilizer.

According to some embodiments, the cream cheese analogue pre-mix composition comprises (i) from about 35 to about 50 wt % BLG; (ii) from about 15 to about 25 wt % of a sugar; and (iii) from about 25 to about 45 wt % of a stabilizer.

According to some embodiments, the cream cheese analogue pre-mix composition comprises (i) from about 70 to about 80 wt % BLG; (ii) from about 15 to about 25 wt % of a sugar; and from about 0.5 to about 1.5 wt % of a stabilizer.

According to some embodiments, the cream cheese analogue pre-mix composition comprises from about 40 to about 75 wt %, from about 45 to about 70 wt %, from about 50 to about 65 wt %, from about 55 to about 60 wt %, from about 35 to about 60 wt %, from about 35 to about 45 wt %, from about 40 to about 55 wt %, from about 45 to about 75 wt %, from about 50 to about 70 wt % or from about 55 to about 65 wt % of BLG. According to some embodiments, the cream cheese analogue pre-mix composition comprises from about 30 to about 55 wt % BLG, from about 30 to about 50 wt % BLG, from about 35 to about 45 wt % or from about 30 to about 45 wt % of BLG.

According to some embodiments, the cream cheese analogue pre-mix composition comprises from about 20 to about 40 wt %, from about 25 to about 35 wt %, from about 20 to about 30 wt %, from about 30 to about 45 wt %, or from about 35 to about 45 wt % of sugar.

According to some embodiments, the cream cheese analogue pre-mix composition comprises from about 1 to about 45 wt %, from about 2 to about 40 wt %, from about 3 to about 35 wt %, from about 5 to about 35 wt %, from about 10 to about 30 wt %, or from about 15 to about 25 wt % of a stabilizer. According to some embodiments, the cream cheese analogue pre-mix composition comprises from about 25 to about 45 wt %, from about 30 to about 40 wt %, from about 35 to about 40 wt %, from about 25 to about 35 wt %, or from about 25 to about 30% of a stabilizer. According to some embodiments, the cream cheese analogue pre-mix composition comprises from 0.5 to 4 wt %, from about 0.5 to about 3.5 wt %, from about 1 to about 3.5 wt %, from about 1 to about 3 wt % or from 1 to 2 wt % of a stabilizer.

According to any one of the above embodiments, the cream cheese analogue pre-mix composition comprises only trace amounts of humidity, and is formulated as a dry powder.

According to some embodiments, the cream cheese analogue pre-mix composition comprises from 30 to 80 wt % of BLG; from 10 to 30 wt % of a sugar; from 0.5 to 45 wt % of a stabilizer; wherein the pre-mix composition is substantially devoid of coagulation minerals, and wherein BLG constitutes at least 51 wt % of the total milk-protein content of the pre-mix composition. According to some embodiments, the cream cheese analogue pre-mix composition comprises from 30 to 55 wt % of BLG and from 20 to 50 wt % of the stabilizer. According to some embodiments, the cream cheese analogue pre-mix composition comprises from 55 to 80 wt % of BLG and from 0.5 to 10 wt % of the stabilizer.

According to some embodiments, the cream cheese analogue pre-mix composition comprises: (i) from about 35% to 50% of BLG protein; (ii) from about 17 to about 25% of sugar; and (iii) from about 30 to about 45% of a stabilizer. According to one embodiment, the sugar is dextrose, and the stabilizer is Etenia 457.

According to some embodiments, the cream cheese analogue pre-mix composition comprises: (i) from 35 to 55% of BLG; (ii) from 1.1 to 2 wt % of trisodium citrate; (iii) from 10 to 30 wt % of sugar; and (iv) from 20 to 50 wt % of starch; wherein the pre-mix composition is substantially devoid of coagulation minerals, and wherein BLG constitutes at least 51 wt % of the total milk-protein content of the pre-mix composition.

According to some embodiments, the cream cheese analogue pre-mix composition comprises: (i) from 30 to 50% of BLG; (ii) from 0.8 to 2 wt % of trisodium citrate; (iii) from 10 to 30 wt % of sugar; (iv) from 20 to 50 wt % of starch; and (v) from 0.5 to 2 wt % of Lcoust bean gum (LGB); wherein the pre-mix composition is substantially devoid of coagulation minerals, and wherein BLG constitutes at least 51 wt % of the total milk-protein content of the pre-mix composition.

According to some embodiments, the firm cream cheese pre-mix composition comprises (i) from 35 to 45 wt % of BLG, (ii) from 0.9 to 1.3 wt % of trisodium citrate, (iii) from 15 to 25 wt % of sugar, and (iv) from 35 to 45 wt % of starch and from 0.6 to 1.22 wt % of Lcoust bean gum (LGB).

According to some embodiments, the cream cheese analogue pre-mix composition comprises: (i) from 65 to 85% of BLG; (ii) from 0.8 to 2 wt % of trisodium citrate; (iii) from 10 to 30 wt % of sugar; and (iv) from 0.5 to 3 wt % of a stabilizer, preferably Lcoust bean gum (LGB); wherein the pre-mix composition is substantially devoid of coagulation minerals, wherein the content of BLG is from 51 to 100 wt % of the protein content of the pre-mix composition, wherein the BLG is coagulated, and wherein the pre-mix composition is for preparation of high-protein firm cream-cheese analogue. According to some embodiments, the cream cheese analogue pre-mix composition comprises from 70 to 80% of BLG, from 0.9 to 1.5 wt % of trisodium citrate, from 20 to 25 wt % of sugar, and from 0.5 to 1.5 wt % of Lcoust bean gum (LGB).

According to any one of the above embodiments, the sugar is selected from the group consisting of a monosaccharide, disaccharide, and polysaccharide. According to some embodiments, the sugar is selected from the group consisting of glucose, fructose, mannose, xylose, arabinose, sucrose, dextrose, maltose, and galactose. According to some embodiments, the sugar is dextrose.

According to any one of the above embodiments, the stabilizer is selected from the group consisting of starch, Lcoust bean gum, and any combination thereof. According to some embodiments, the stabilizer is Etenia 457. In some examples, the stabilizer comprises starch or starch derivative, e.g., potato starch that was modified through enzymatic treatment. An example of a commercially available stabilizer that can be utilized in the non-dairy product as disclosed herein is known by the brand name Etenia™ (known as an E number free starch), e.g., Etenia 457.

According to some embodiments, the cream cheese analogue pre-mix composition further comprises a divalent ion chelator. According to some embodiments, the chelator is trisodium citrate.

According to some embodiments, the pre-mix composition disclosed herein comprises a stabilizer. In some examples, the stabilizer is one selected to provide the non-dairy product produced from the pre-mix composition (as described and exemplified hereinbelow) with an emulsifying effect. In some examples, the stabilizer is one having a thickening effect. In some examples, the stabilizer is one having hydrocolloid functionality. A benefit of using a stabilizer having any one or combination of the above functionalities resides in providing the eventual non-dairy product with a rich and creamy mouthfeel.

According to some embodiments, the pre-mix composition comprises a rBLG protein, a bulking agent, and a sweetener. According to some embodiments, the pre-mix composition comprises a rBLG protein, a bulking agent, and a saccharide-containing molecule. According to some embodiments, the pre-mix composition comprises a rBLG protein, a bulking agent, and a stabilizer.

The pre-mix composition can be provided in various physical forms. According to some embodiments, the pre-mix composition is solid. In some examples, the pre-mix composition is provided as a dry powder.

The amount of the BLG protein in the pre-mix composition can vary, depending on the eventually produced non-dairy product. In some examples, the amount of the BLG protein in the pre-mix composition is between 10 wt % and 80 wt % or between 20 to 60 wt %. In some examples, the amount of the BLG protein in the cream cheese pre-mix composition is between 30 to 60 wt %. In some examples, the amount of the BLG protein in the pre-mix composition is between 10 wt % and 80 wt % or between 35 to 55 wt %. In some examples, the amount of the BLG protein in the cream cheese pre-mix composition is between 40 wt % and 50 wt %. In some examples, the amount of the BLG protein in the cream cheese pre-mix composition is between 40 wt % and 45 wt %. In some examples, the amount of the BLG protein in the cream cheese pre-mix composition is between 41 wt % and 43 wt %. In some examples, the amount of the BLG protein is at least 35 wt %. In some examples, the amount of the BLG protein is at least 40 wt %. In some examples, the amount of the BLG protein is at least 41 wt %. In some examples, the amount of the BLG protein is at least 41.3 wt %. According to some embodiments, the BLG is rBLG. According to some embodiments, the BLG is rBLG isolate. According to some embodiments, the BLG is rBLG protein of the present invention.

It has been found that such a high concentration of BLG protein in the pre-mix composition allows for the formation of a non-dairy final, consumable product with more than 2 wt % BLG protein, at times more than even 3 wt % BLG protein out of the total weight of the product (wet weight).

It has been found that the dairy analogue food product comprising the rBLG isolate of the present disclosure is lacking an aftertaste that is typically ascribed with hitherto known recombinant (e.g., yeast derived) rBLG concentrates or isolates and edible products prepared therefrom.

Further, it has been found that even when using more than 3 wt % of the rBLG in the non-dairy edible product, such as the non-dairy cream cheese exemplified hereinbelow, there is no aftertaste usually exhibited with yeast and/or plant-based/derived protein isolates.

In some embodiments, the weight ratio of a bulking agent to BLG protein in the cream cheese pre-mix composition is between about 1:5 and about 1:1. In some examples, the weight ratio of a bulking agent to BLG protein in the cream cheese pre-mix composition between about 1:3 and about 1:1.5. In some examples, the bulking agent to BLG protein weight ratio is between about 1 to 2. In some embodiments, the weight ratio of a bulking agent to BLG protein in the pre-mix composition is about 1:2.

In some embodiments, the cream cheese pre-mix composition comprises from 5 to 40 wt % of the bulking agent. According to some embodiments, the cream cheese pre-mix composition comprises from 10 to 35 wt %, from 15 to 30 wt %, from 10 to 30 wt % or from 15 to 25 wt % of the bulking agent. According to some embodiments, the bulking agent is a saccharide. According to some embodiments, the bulking agent is dextrose.

In some examples, the weight ratio of a stabilizer to BLG in the cream cheese pre-mix composition protein is between about 0.3 and 1:3. In some examples, the weight ratio of a stabilizer/texturizer to BLG in the cream cheese pre-mix composition protein is between about 0.6 and 1:2. In some examples, the weight ratio of a stabilizer/texturizer to BLG in the pre-mix composition protein is between about 0.6 and 1:1.5. In some examples, the weight ratio of a stabilizer/texturizer to BLG in the cream cheese pre-mix composition protein between about 1:1 and 1:2. In some examples, the weight ratio of a stabilizer/texturizer to BLG in the pre-mix composition protein is between about 0.8 and 1:1.1 or about 0.9.

In some embodiments, the cream cheese analogue pre-mix composition comprises from 20 to 60 wt % of the stabilizer. According to some embodiments, the cream cheese pre-mix composition comprises from 25 to 55 wt %, from 30 to 50 wt %, from 35 to 45 wt % of the stabilizer. In some embodiments, the cream cheese pre-mix composition comprises from 36 to 40 wt % or about 38.5 wt % of the stabilizer. According to some embodiments, the stabilizer is a functional enzymatically treated potato starch. According to some embodiments, the stabilizer is Etenia.

In some examples, the cream cheese analogue pre-mix composition comprises or consists essentially of the BLG protein in an amount of between about 35 wt % and 45 wt %; a bulking agent in an amount of between 15 to 25 wt % or about 17 wt % and 22 wt %; and a stabilizer in an amount of between 30 wt % and 45 wt %.

In some examples, the cream cheese analogue pre-mix composition comprises or consists essentially of the BLG protein in an amount of between about 40 wt % and 50 wt %; a bulking agent in an amount of between 15 to 25 wt % or about 17 wt % and 22 wt %; and a stabilizer in an amount of between 30 wt % and 45 wt %.

In some examples, the cream cheese analogue pre-mix composition comprises or consists essentially of the BLG protein in an amount of between about 40 wt % and 50 wt %; dextrose in an amount of between 17 wt % and 22 wt %; and potato starch in an amount of between 30 wt % and 40 wt %. According to some embodiments, the potato starch is Etenia.

In some examples, the cream cheese analogue pre-mix composition comprises or consists essentially of the BLG protein in an amount of 40-42 wt %; dextrose in an amount of between 20 wt %-21 wt %; and potato starch in an amount of between 38 wt % and 40 wt %. According to some embodiments, the potato starch is Etenia.

The pre-mix compositions provided herein can be packed as a kit-of-parts, where each component is packed separately, and the kit optionally comprises instructions on how to combine the parts into the pre-mix composition.

As stated above, BLG isolated from milk may be used instead of rBLG, and vice versa. Therefore, according to any one of the above embodiments, the rBLG may be replaced by BLG isolated from milk and the resulted pre-mix composition is a non-animal-free pre-mix composition. According to some embodiments, a combination of BLG isolated from milk and rBLG is used.

According to some embodiments, the present invention provides a cream cheese analogue pre-mix composition comprising (i) from 35 to 55 wt %; (ii) from 40 to 50% of rBLG; (iii) from 1.1 to 2 wt % of trisodium citrate; (iv) from 10 to 30 wt % of sugar; and (v) from 20 to 50 wt % of starch as a stabilizer. According to some embodiments, the cream-cheese analogue pre-mix composition comprises (i) from 30 to 50 wt % of rBLG; (ii) from 1.1 to 2 wt % of trisodium citrate; (iii) from 10 to 30 wt % of sugar; and (iv) from 20 to 50 wt % of starch as a stabilizer. According to some embodiments, the cream-cheese analogue pre-mix composition comprises from 40 to 50 wt % of the rBLG. According to some embodiments, the cream cheese analogue pre-mix composition comprises from 42 to 48 wt % of the rBLG. According to some embodiments, the cream-cheese analogue pre-mix composition comprises from 35 to 45 wt % or from 37 to 47 wt % of the rBLG protein. According to some embodiments, the cream cheese analogue pre-mix composition comprises from 1.3 to 1.7 wt % of trisodium citrate. According to some embodiments, the sugar is dextrose. According to other embodiments, the cream cheese analogue pre-mix composition comprises from 20 to 25 wt % of sugar such as dextrose. According to other embodiments, the cream-cheese analogue pre-mix composition comprises from 25 to 35 wt % of starch. According to some embodiments, the cream-cheese analogue pre-mix composition comprises (i) about 45.5% of rBLG isolate comprising at least 85 wt % of rBLG; (ii) about 1.3 wt % of trisodium citrate; (iii) about 22.5 wt % of dextrose, and (iv) about 30 wt % of starch as a stabilizer.

According to some embodiments, the present invention provides a firm cream-cheese analogue pre-mix composition comprising (i) from about 30 to about %; (ii) from about 35 to about 45% of BLG; (iii) from about 0.8 to about 2 wt % of trisodium citrate; (iv) from about 10 to about 30 wt % of sugar; (v) from about 20 to about 50 wt % of starch as a stabilizer; and (vi) from about 0.5 to about 2 wt % of Lcoust bean gum (LGB) as a stabilizer. According to some embodiments, the cream-cheese analogue pre-mix composition comprises (i) from about 32 to about 45 wt % of BLG; (ii) from about 0.9 to about 1.2 wt % of trisodium citrate; (iii) from about 15 to about 25 wt % of sugar; (iv) from about 30 to about 50 wt % of starch; and (v) from about 0.7 to about 1 wt % of LGB. According to some embodiments, the sugar is dextrose. According to some embodiments, the cream cheese analogue pre-mix composition comprises from about 35 to about 50 wt % of the BLG. According to some embodiments, the cream cheese analogue pre-mix composition comprises from about 42 to about 48 wt % of the BLG. According to some embodiments, the cream cheese analogue pre-mix composition comprises from about 25 to about 40 wt % or from about 28 to about 35 wt % of the BLG. According to some embodiments, the cream cheese analogue pre-mix composition comprises from about 1 to about 1.2 wt % of trisodium citrate. According to other embodiments, the cream cheese analogue pre-mix composition comprises from about 20 to about 25 wt % of sugar such as dextrose. According to other embodiments, the cream cheese analogue pre-mix composition comprises from about 35 to about 45 wt % of starch. According to some embodiments, the cream cheese analogue pre-mix composition comprises (i) about 38.5% of BLG; (ii) about 1.1 wt % of trisodium citrate; (iii) about 18.8 wt % of dextrose; (iv) about 41 wt % of starch; and (v) about 0.85 wt % of LBG. According to some embodiments, the BLG is rBLG, e.g., rBLG of the present invention.

According to some embodiments, the present invention provides a high protein firm cream cheese analogue pre-mix composition comprising (i) from about 60 to about 90%, or from about 65 to about 85% of BLG; (ii) from about 0.8 to about 2 wt % of trisodium citrate; (iii) from about 10 to about 30 wt % of sugar; and (iv) from about 0.5 to about 3 wt % of Lcoust bean gum (LGB) as a stabilizer. According to some embodiments, the cream cheese analogue pre-mix composition comprises from about 70 to about 80 wt % of BLG; from about 0.9 to about 1.4 wt % of trisodium citrate; from about 15 to about 25 wt % of sugar; and from about 0.7 to about 1.2 wt % of LGB. According to some embodiments, the sugar is dextrose. According to some embodiments, high protein firm cream cheese analogue pre-mix composition comprises from about 71 to about 79 wt % of the BLG. According to some embodiments, the cream cheese analogue pre-mix composition comprises from about 72 to about 78 wt % of the BLG. According to some embodiments, the high protein firm cream cheese analogue pre-mix composition comprises from about 50 to about 70 wt % or from about 58 to about 68 wt % of the BLG. According to some embodiments, the cream cheese analogue pre-mix composition comprises from about 1.2 to about 1.4 wt % of trisodium citrate. According to other embodiments, the cream cheese analogue pre-mix composition comprises from about 20 to about 25 wt % of sugar such as dextrose. According to other embodiments, the cream cheese analogue pre-mix composition comprises from about 0.5 to about 1.5 wt % of LBG. According to some embodiments, the cream cheese analogue pre-mix composition comprising about 75% of BLG, about 1.3 wt % of trisodium citrate, about 22.5 wt % of dextrose, and about 1 wt % of LBG.

According to any one of the above embodiments, BLG constitutes at least 53 wt %, at least 55 wt %, at least 60 wt %, at least 65 wt %, at least 70 wt %, at least 75 wt %, at least 80 wt %, at least 85 wt %, at least 90 wt %, at least 95 wt %, at least 97 wt %, at least 98% or at least 99% of the total milk-protein content of the cream cheese analogue pre-mix composition. According to any one of the above embodiments, the BLG constitutes from about 51 wt % to about 100 wt %, from about 55 wt % to about 95 wt %, from 60 wt % to about 90 wt %, from 65 wt % to about 85 wt %, from 70 wt % to about 80 wt %, from 70 wt % to about 99 wt %, from 75 wt % to about 95 wt %, from 85 wt % to about 95 wt %, from 90 wt % to about 100 wt % or from 95 wt % to about 99 wt % of the total milk-protein content of the cream cheese analogue pre-mix composition. According to some embodiments, the BLG is the sole milk protein in the cream cheese analogue pre-mix composition. According to any one of the above embodiments, BLG constitutes at least 51 wt %, at least 53 wt %, at least 55 wt %, at least 60 wt %, at least 65 wt %, at least 70 wt %, at least 75 wt %, at least 80 wt %, at least 85 wt %, at least 90 wt %, at least 95 wt %, at least 97 wt %, at least 98% or at least 99 wt % of the total protein content of the cream cheese analogue pre-mix composition. According to some embodiments, BLG constitutes from about 51 wt % to about 100 wt %, from about 55 wt % to about 99 wt %, from about 60 wt % to about 98 wt %, from about 65 wt % to about 97 wt %, from about 70% to about 95 wt %, from about 75 wt % to about 93 wt %, from about 80 wt % to about 90 wt %, from about 60 wt % to about 80 wt %, from about 55 wt % to about 95 wt %, from 60 wt % to about 90 wt %, from about 65 wt % to about 85 wt %, from about 70 wt % to about 80 wt %, from 70 wt % to about 99 wt %, from 75 wt % to about 95 wt %, from 85 wt % to about 95 wt %, from 90 wt % to about 100 wt % or from 95 wt % to about 99 wt %, or about 100 wt % of the total protein content of the cream cheese analogue pre-mix composition. According to some embodiments, the BLG is a rBLG. According to some embodiments, the BLG is the rBLG of the present invention.

According to other embodiments, the final food product may comprise compounds derived from milk and/or non-animal-free premix composition. In such a case, the food product is a non-animal-free food product.

To produce the cream cheese analogue, the cream cheese pre-mix composition is hydrated and combined with other ingredients such as fat or fat alternative, typically under a temperature at which the fat or fat alternative is fluid. In some examples, the fat or fat alternatives are non-animal fat or fat alternative. The terms “fat alternative” and “fat substitute” are used herein interchangeably and refer to a food product or component with generally the same functions, stability, physical, and chemical characteristics as regular animal fat.

### Methods of Preparing Cream Cheese Analogues

According to another aspect, the present invention provides a method of preparing a cream cheese analogue comprising a coagulated BLG, the method comprising:
(i) mixing (a) water, (b) from about 5 to about 15 wt % of the cream cheese analogue premix composition according to any one of the above embodiments or the content thereof, and (c) a lipid;
(ii) pasteurizing the composition of step (i)
(iii) optionally, cooling down the composition of step (ii);
(iv) adding a coagulation mineral to the composition step (iii), or of step (ii), if step (iii) is absent, thereby coagulating the BLG protein; and
(v) subjecting the composition of step (iv) to an acidification;
(vi) optionally, adding a flavoring salt to the composition of step (v); and
(vii) optionally, smoothing the composition of step (iv) or step (v) or step (vi),
wherein BLG constitutes at least 51 wt % of the total milk-protein content of the cream cheese analogue.

All terms, embodiments and definitions defined in any one of the above aspects apply and are encompassed herein as well.

It is understood and contemplated herein in any one of the embodiments of the present invention that adding a pre-mix composition of the present invention has the meaning of adding the content of the pre-mix composition as well as adding the components/constituents of the pre-mix composition.

According to some embodiments, the method comprises step (ii). According to some embodiments, the method comprises step (vi). According to some embodiments, the method comprises step (vii). According to some embodiments, the method comprises steps (vi) and (vii).

According to some embodiments, the resulted dairy analogue food product comprises from about 0.0015 to about 0.35 wt % of a coagulation mineral(s). According to some embodiments, the resulted dairy analogue food product comprises from about 0.002 to about 0.30 wt % of a coagulation mineral(s). According to some embodiments, the resulted dairy analogue food product comprises from about 0.005 to about 0.25 wt % of a coagulation mineral(s). According to some embodiments, the resulted dairy analogue food product comprises from about 0.01 to about 0.20 wt % of a coagulation mineral(s). According to some embodiments, the resulted dairy analogue food product comprises from about 0.05 to about 0.2 wt % of a coagulation mineral(s). According to some embodiments, the resulted dairy analogue food product comprises from about 0.1 to about 0.25 wt % of a coagulation mineral(s). According to some embodiments, the coagulation mineral is calcium. According to some embodiments, the coagulation mineral salt is calcium chloride.

Acidification can be carried out using Lactic Acid Bacteria (LAB) or by using a whey culture. The LAB bacteria can be a mixed-strain or defined-strain cultures. In some examples, the bacteria culture is mesophilic. In some other examples, the bacteria culture is thermophilic. Acidification may be carried out by adding acid.

According to some embodiments, the composition of step (i) is a homogenized composition.

According to any one of the above embodiments, the BLG is rBLG, e.g., rBLG of the present invention.

According to some embodiments, the method of preparation of cream cheese analogue comprises the steps of:
(i) mixing the cream cheese pre-mix composition of the present invention with preheated water and slowly agitating the mixture for at least 45 minutes;
(ii) adding from about 15 to 35 wt % plant oil and agitating under a moderate agitation;
(iii) heating the mixture to at least 60° C. and double-stage homogenizing at 50 and then at 200 bar;
(iv) pasteurizing to 85° C. for 2 minutes and cool down to 24-32° C.;
(v) transferring into a fermenter under slow agitation;
(vi) adding about 0.05 to about 0.3 wt % calcium chloride and 1 U/10 L of mesophilic lactic acid bacteria;
(vii) arresting agitation;
(viii) fermenting the mixture until reaching pH 4.1-4.9;
(ix) adding from 0.5 to 1.5 wt % flavoring salt under moderate agitation; and
(x) filing into the final container.

According to some embodiments, the cream cheese analogue premix composition comprises between about 40 wt % and about 50 wt % of BLG; dextrose in an amount of between about 17 wt % and about 22 wt %; and potato starch in an amount of between about 30 wt % and about 40 wt %. According to one embodiment, the BLG is rBLG.

A general procedure for preparing the cream cheese analogue of the present invention is provided. The content of the cream cheese analogue pre-mix and or of the cream cheese analogue as well as some of the conditions may vary. However, they all fall under the general procedure described hereinbelow. In some examples, the cream cheese analogue is obtained by a method comprising:
introducing into an aqueous suspension of a cream cheese analogue pre-mix composition disclosed herein an amount of fat or fat alternative, the aqueous suspension and the fat or fat alternative being at temperatures at which the fat or fat alternative is fluid, to form an integrated mixture;
homogenizing and optionally pasteurizing the integrated mixture;
introducing at least one fermentation bacterium and supporting salts into the integrated mixture to form a fermentation mixture; and
allowing fermentation of the fermentation mixture until reaching acidic pH to obtain the cream cheese analog.

According to some embodiments, the coagulation minerals are added together with the fermentation bacterium.

In some examples, the method comprises mixing the pre-mix composition with an aqueous solution at a temperature of between about 40° C. and about 60° C., preferably at about 40° C., to form the aqueous suspension.

In some examples, the aqueous suspension is agitated for at least 10 min, at times, at least 20 min, at times, at least 30 min, before introducing thereto the fat or fat alternative.

In the context of the present disclosure, when referring to fat or fat alternative it is to be understood as encompassing plant derived fats or oils, such as coconut oil, shea oil, etc. In some examples, the fat or fat alternative comprises or consists of coconut oil and/or refined coconut oil. The introduction of fat or fat alternative is typically performed during agitation or mixing of the aqueous suspension.

The resulting integrated mixture comprises the protein and the fat material. This integrated mixture is homogenized under conditions known in the art of homogenization. For example, and without being limited thereto, the integrated mixture is double-staged homogenized first at about 60° C. at about 250 Bar, and then again (under different conditions) after the bacteria-based fermentation.

In some examples, the homogenized mixture is subjected also to pasteurization. Pasteurization can be performed by any known technique and under acceptable conditions. For example, pasteurization is performed at 90° C. for several minutes, after which the pasteurized mixture is cooled down.

The homogenized and optionally pasteurized mixture is then subjected to fermentation. To this end, the homogenized and optionally pasteurized mixture is added with at least one type of fermentation bacterium. In some examples, the fermentation is performed in the final containers, without a second homogenization step. In some examples, the fermentation is performed before moving the product to the final containers, and a second homogenization step is performed before moving the product to the final containers.

In some examples, the fermentation bacterium is one that is typically used for fermentation in the dairy industry. Examples of such bacteria include, without being limited thereto, lactic acid bacteria, specifically mesophilic lactic acid bacteria, such as CHN 19.

For example, and without being limited thereto, the integrated mixture is double-staged homogenized, the second time at about 30-65° C. at about 50-100 Bar after the bacteria-based fermentation.

To facilitate fermentation by the added fermentation bacterium, the mixture is also supplemented with at least one salt, such as those known in the food industry to be used as firming agents. For example, the salt can be calcium chloride and/or sodium chloride.

The fermented product can then be transferred to containers, under procedures known in the art.

In some examples, the non-dairy cream cheese analogue disclosed herein comprises or consists essentially of BLG protein in an amount of between 9 wt % and 12 wt %, non-animal fat material in an amount of between 20 wt % and 30 wt %; food acceptable additives (e.g., firming agents, preservatives, thickeners) and water. According to some embodiments, the non-dairy cream cheese analogue comprises lactic acid bacteria or traces or debris thereof.

In some examples, the cream cheese analogue disclosed herein comprises or consists essentially of rBLG protein in an amount of between 9 wt % and 12 wt %, fat material in an amount of between 20 wt % and 30 wt %; food acceptable additives (e.g., firming agents, preservatives, thickeners) and water. According to some embodiments, the non-dairy cream cheese further comprises impurities obtained from pre-mixture composition. According to some embodiments, the cream cheese analogue comprises bacteria such as lactic acid bacteria or traces of such bacteria. According to some embodiments, the cream cheese comprises salts such as sodium chloride and calcium chloride. According to some embodiments, the cream cheese comprises from 7 to 15 wt % of rBLG isolate, from 15 to 35 wt % of plant oil, from 0.5 to 1.5 wt % of sodium chloride, from 0.05 to 0.3 wt % of calcium chloride, from 0.05 to 0.3 wt % of a preservative, e.g., potassium sorbate, optionally 0.05 to 0.15 U/ml of mesophilic lactic acid bacteria or traces of the bacteria, and water. According to some embodiments, the cream cheese analogue comprises from 9 to 12 wt % of rBLG protein, from 20 to 30 wt % of plant oil, from 0.8 to 1 wt % of sodium chloride, from 0.1 to 0.2 wt % of calcium chloride, from 0.075 to 0.15 wt % of a preservative, e.g., potassium sorbate, optionally 0.075 to 0.125 U/ml of mesophilic lactic acid bacteria or traces of the bacteria, and water. According to some embodiments, the cream cheese analogue comprises about 10.5 wt % of rBLG protein, about 25 wt % of plant oil, about 0.9 wt % of sodium chloride, about 0.15 wt % of calcium chloride, about 0.1 wt % of potassium sorbate, optionally about 0.1 U/ml of mesophilic lactic acid bacteria or traces of the bacteria, and water. According to any one of the above embodiments, the cream cheese analogue has a pH of from 4 to 5.5, from 4.5 to 5 or about 4.7. According to some embodiments, the non-dairy cream cheese analogue comprises lactic acid bacteria or traces or debris thereof.

According to any one of the above embodiments, the BLG is the rBLG protein or the rBLG isolate of the present invention. According to any one of the above embodiments, the edible product comprising a rBLG protein or the rBLG isolate of the present disclosure lacks an aftertaste that is typically ascribed with hitherto known recombinant (e.g., yeast derived) rBLG concentrates or isolates and edible products prepared therefrom. According to some embodiments, the non-dairy cream cheese of the present invention lacks the aftertaste usually exhibited with yeast and/or plant-based/derived protein isolates.

According to some embodiments, the present disclosure provides a method for preparing a cheese analogue product, the method comprises: (i) providing a starting protein composition comprising at least 1 wt % BLG out of a total weight of the starting protein composition, the composition comprising sub-critical amount of coagulation minerals; (ii) subjecting the starting protein composition to homogenization to obtain a homogenized composition; (iii) subjecting the homogenized composition to pasteurization to obtain pasteurized composition; and (iv) adding to the pasteurized composition at least one coagulation mineral whereby the pasteurized composition coagulates into a first cheese analogue product.

According to any one of the above and below embodiments, BLG constitutes at least 51 wt % of the total milk-protein content of the cream cheese analogue. According to some embodiments, BLG constitutes at least 55 wt % of the total milk-protein content of the cream cheese analogue. According to some embodiments, BLG constitutes at least 60 wt %, at least 65 wt %, at least 70 wt %, at least 75 wt %, at least 80 wt %, at least 85 wt %, at least 90%, at least 95 wt %, at least 97 wt %, at least 98 wt % or at least 99 wt % of the total milk-protein content of the cream cheese analogue. According to any one of the above embodiments, the BLG constitutes from about 51 wt % to about 100 wt %, from about 55 wt % to about 95 wt %, from 60 wt % to about 90 wt %, from 65 wt % to about 85 wt %, from 70 wt % to about 80 wt %, from 70 wt % to about 99 wt %, from 75 wt % to about 95 wt %, from 85 wt % to about 95 wt %, from 90 wt % to about 100 wt % or from 95 wt % to about 99 wt % of the total milk-protein content of the cream cheese analogue. According to any one of the above and below embodiments, BLG constitutes at least 51 wt % of the total protein content of the cream cheese analogue. According to some embodiments, BLG constitutes at least 51 wt %, at least 53 wt %, or at least 55 wt % of the total protein content of the cream cheese analogue. According to some embodiments, BLG constitutes at least 60 wt %, at least 65 wt %, at least 70 wt %, at least 75 wt %, at least 80 wt %, at least 85 wt %, at least 90 wt %, at least 95 wt %, at least 97 wt %, at least 98 wt % or at least 99 wt % of the total protein content of the cream cheese analogue. According to some embodiments, BLG constitutes from about 51 wt % to about 100 wt %, from about 55 wt % to about 99 wt %, from about 60 wt % to about 98 wt %, from about 65 wt % to about 97 wt %, from about 70% to about 95 wt %, from about 75 wt % to about 93 wt %, from about 80 wt % to about 90 wt %, from about 60 wt % to about 80 wt %, from about 55 wt % to about 95 wt %, from 60 wt % to about 90 wt %, from about 65 wt % to about 85 wt %, from about 70 wt % to about 80 wt %, from 70 wt % to about 99 wt %, from 75 wt % to about 95 wt %, from 85 wt % to about 95 wt %, from 90 wt % to about 100 wt % or from 95 wt % to about 99 wt %, or about 100 wt % of the total protein content of the cream cheese analogue.

In some examples, the at least one protein is artificially obtained by recombinant methods. Thus, the at least one artificially obtained protein is a recombinant protein that is produced using protein expression technologies. The host for such technologies can be, without being limited thereto, any one or a combination of bacterial cells, eukaryote, yeast, insect cells, fungal cells, plant cells.

In some examples, the cheese analogue is “animal-free”. Such animal-free cheese analogue, comprising rBLG, is denoted also as a recombinant cheese analogue.

In some other examples, the at least one protein of the cheese analogue is artificially obtained by isolation from milk. In yet some other examples, the at least one protein of the cheese analogue is artificially obtained by synthetic methods. In some examples, the cheese analogue comprises isolated, e.g., milk-isolated, BLG, optionally as the sole protein. In some examples, the cheese analogue comprises recombinant BLG (rBLG) of the present invention, optionally as the sole protein or as the sole milk protein.

In some examples, the method of producing the disclosed recombinant cheese analogue products comprises as a first step the providing of a starting protein composition comprising at least 1 wt % BLG (out of a total weight of the starting protein composition).

In some examples, the starting protein composition comprises at least 2 wt % BLG. In some examples, the starting protein composition comprises at least 3 wt % BLG. In some examples, the starting protein composition comprises at least 4 wt % BLG. In some examples, the starting protein composition comprises at least 5 wt % BLG. In some examples, the starting protein composition comprises at least 6 wt % BLG. In some examples, the starting protein composition comprises at least 7 wt % BLG. In some examples, the protein starting composition comprises at least 8 wt % BLG. In some examples, the starting protein composition comprises at least 9 wt % BLG. According to some embodiments, the BLG is rBLG.

In some examples, the starting protein composition comprises at most 10 wt % BLG. In some examples, the starting protein composition comprises at most 9 wt % BLG. In some examples, the starting protein composition comprises at most 8 wt % BLG. In some examples, the starting protein composition comprises at most 7 wt % BLG. In some examples, the starting protein composition comprises at most 6 wt % BLG. In some examples, the starting protein composition comprises at most 5 wt % BLG. According to some embodiments, the BLG is rBLG.

In some examples, the starting protein composition comprises between 1 wt % and 10 wt % BLG. In some examples, the starting protein composition comprises between 2 wt % BLG and 9 wt % BLG. In some examples the starting protein composition comprises between 3 wt % BLG and 8 wt % BLG. In some examples, the starting protein composition comprises between 4 wt % BLG and 7 wt % BLG. In some examples, the starting protein composition comprises between 5 wt % and 6 wt %, from 5.1 to 5.9, from 5.2 to 5.8, from 5.3 to 5.7, from 5.4 to 5.6, from 5.3 to 6, from 5.4 to 5.8 wt % of BLG. According to some embodiments, the BLG is rBLG.

In some examples, the starting protein composition comprises between 1 wt % and 5.5 wt % BLG. In some examples, the starting protein composition comprises between 1 wt % and 5.4 wt % BLG. In some examples, the starting protein composition comprises between 1 wt % and 5.3 wt % BLG. In some examples, the starting protein composition comprises between 1 wt % and 5.2-wt % BLG. In some examples, the starting protein composition comprises between 1 wt % and 5.1 wt % BLG. In some examples, the starting protein composition comprises between 1 wt % and 5.0 wt % BLG. According to some embodiments, the BLG is rBLG. In some examples, the starting protein composition comprises 5 wt %±1 wt % BLG. According to some embodiments, the starting protein composition can comprise additional proteins. In some examples, the additional protein is a milk protein. According to some embodiments, the milk protein is animal free. In some examples, the additional protein is a different whey protein. According to any one of the above embodiments, BLG constitutes at least 51 wt % of the total milk-protein content of the cream cheese analogue.

In some examples, the additional protein in the context of the present disclosure can be any such protein recombinantly produced and/or synthetically produced. Further, when referring to additional protein that is whey or milk protein, it is to be understood as encompassing any member of the group consisting of casein, serum albumin, and α-lactoalbumin (ALA), and combinations thereof.

In some examples, the additional protein is casein. When referring to casein it is to be understood to include any one or combination of α-casein (this includes independently α1S-casein, α2S-casein, and any combination of α1S-casein and α2S-casein), β-casein, γ-casein.

In some examples, the additional protein is serum albumin, such as bovine serum albumin. In some examples, the additional protein is α-lactoalbumin.

In some examples, the starting protein composition also comprises lipids.

In the context of the present disclosure, when referring to lipids it is to be understood as encompassing oil and/or fat. In this connection, oils are considered to encompass lipids that are liquid or semi-solid at room temperature, and typically (although not exclusively) plant derived; while fats are considered to encompass lipids that are solid at room temperature and typically (although not exclusively) animal derived.

In the context of the present disclosure, when referring to lipids it is to be understood as encompassing edible lipids or edible lipids alternatives. In some examples, the protein composition is low in trans fatty acids.

In some examples, the starting protein composition comprises at least 1 wt % edible lipids. In some examples, the starting protein composition comprises up to 40 wt % edible lipids. In some examples, the starting protein composition comprises between 1 wt % and 40 wt % edible lipids. In some examples, the starting protein composition comprises between 5 wt % and 35 wt % edible lipids. In some examples, the starting protein composition comprises between 10 wt % and 30 wt % edible lipids. In some examples, the starting protein composition comprises between 15 wt % and 25 wt % edible lipids. In some examples, the starting protein composition comprises between about 5% and about 20% edible lipids.

In some examples, the starting protein composition comprises up to 30 wt % edible lipids. In some examples, the starting protein composition comprises up to 20 wt % edible lipids. In some examples, the starting protein composition comprises up to 10 wt % edible lipids. In some examples, the starting protein composition comprises up to 5 wt % edible lipids. In some examples, the starting protein composition comprises at least 5 wt % edible lipids. In some examples, the starting protein composition comprises at least 10 wt % edible lipids. In some examples, the starting protein composition comprises at least 20 wt % edible lipids. In some examples, the starting protein composition comprises at least 30 wt % edible lipids. The terms “at most” and “up to” are used herein interchangeably.

In some examples, the lipids comprise triglycerides. In some examples, the lipids comprise oils. Non limiting examples for vegetable oils are shea oil, sunflower oil, coconut oil, rapeseed oil, nut oil, palm oil, kernel oil, olive oil, soya oil, cotton oil, and cocoa butter (Theobroma oil). In one specific example, the oil is shea oil. In some examples, the lipids are recombinantly produced. Such lipids can be produced using a recombinant host cell (which can be a plant cell or microbial cell) that have been genetically modified to modulate the production or activity of proteins that are involved in the biosynthesis of lipids. In some examples, the host cell is an oleaginous cell. In some examples, the lipids are milk lipids that are recombinantly produced.

In some examples, the starting protein composition comprises at least one sugar. In some examples, the starting protein composition comprises at least one sugar as a bulking agent.

In some examples, the sugar comprises a saccharide-containing molecule. In some examples, the saccharide-containing molecule is a monosaccharide, a disaccharide, or a polysaccharide.

In some examples, the sugar is a monosaccharide selected from the group consisting of sucrose, glucose, dextrose (D-glucose), fructose, mannose, maltose, xylose, arabinose, galactose, and any combination thereof. In some examples, the sugar comprises or is dextrose. In some examples, the sugar is not lactose. According to some embodiments, the protein composition comprises from 1 to 10 wt % of sugar. According to some embodiments, the protein composition comprises from 1 to 5 wt % or from 1 to 3 wt % or sugar. According to some embodiments, the sugar is dextrose.

The starting protein composition is prepared by mixing the at least BLG protein with an edible lipid and the sugar within water until an emulsion is formed.

In some examples, mixing the BLG protein with an edible lipid and sugar is at a temperature of at least 30° C., at times at least 40° C. In some examples, mixing the BLG protein with an edible lipid and sugar is at a temperature of up to 70° C., at times up to 60° C., or up to 50° C. In some examples, mixing the BLG protein with an edible oil and sugar is at a temperature of between about 30° C. and about 70° C., between about 40° C. and 60° C., or between about 30° C. and about 50° C.

According to some embodiments, the starting protein composition comprises from 1 to 10 wt % of BLG, from 10 to 40 wt % of lipids, and from 1 to 10 wt % of sugar. According to some embodiments, the starting protein composition comprises from 3 to 8 wt % of BLG such as rBLG, from 15 to 30 wt % of oil, such as Shia oil, and from 1 to 5 wt % of sugar.

In some examples, the mixture is homogenized under conditions that allow the formation of fat globules therein. In some examples, homogenization is carried out until the fat globules are reduced in size to less than 2 micrometers in diameter. In some examples, the mixture or the resulting homogenized composition is subjected to pasteurization to obtain a pasteurized composition. In some examples, during pasteurization, the composition or homogenized composition is mildly heated, typically at a temperature of less than 100° C., at times, less than 90° C.; at times, less than 80° C. In some examples, during pasteurization, the composition or homogenized composition is mildly heated, typically at a temperature between 50° C. and 100° C.; at times at a temperature of between 50° C. and 90° C., or at a temperature of between 50° C. and 80° C. According to some embodiments, the composition or homogenized composition is heated at a temperature of between 85° C. and 95° C. In some examples, pasteurization is carried out at a temperature of about 90° C. for several minutes. In some examples, pasteurization is carried at a temperature range of 85° C. and 95° C., at times, at a temperature range of between about 87° C. and 93ºC. the duration is typically between 1 and about 10 minutes, at times, between about 2 and 9 minutes, at times between about 2 and 8 minutes, at times between about 3 and 7 minutes or at times for about 5 minutes, followed by cooling the pasteurized composition to a temperature below 50° C., or below 40° C., or below 35° C., e.g., about 30° C. The temperature and duration of pasteurization is determined such that only partial protein denaturation takes place. In some embodiments, the composition is a starting protein composition.

In some examples, the starting protein composition comprises not more than 0.009 wt % of coagulation minerals. In some examples, the starting protein composition comprises not more than 0.008 wt % of coagulation minerals. In some examples, the starting protein composition comprises not more than 0.007 wt % of coagulation minerals. In some examples, the starting protein composition comprises not more than 0.006 wt % of coagulation minerals. In some examples, the starting protein composition comprises not more than 0.005 wt % of coagulation minerals. In some examples, the starting protein composition comprises not more than 0.004 wt % of coagulation minerals. In some examples, the starting protein composition comprises not more than 0.003 wt % of coagulation minerals. In some examples, the starting protein composition comprises not more than 0.002 wt % of coagulation minerals. In some examples, the starting protein composition comprises not more than 0.001 wt % of coagulation minerals. According to some embodiments, the starting protein composition is substantially devoid of minerals and more specifically of coagulation minerals as defined above. According to some embodiments, the starting protein composition is substantially devoid of divalent cations.

Yet, coagulation is required for the production of cheese analogues. Thus, in accordance with the present disclosure, at least one mineral such as coagulation mineral(s) (as defined hereinabove) is added after pasteurization to cause, initiate and/or promote coagulation.

In some examples, the coagulation minerals comprise a member of the group consisting of calcium, copper, iron, magnesium, manganese phosphorous, potassium, selenium, sodium, and zinc. In some examples, the coagulation mineral is any one or combination of calcium, magnesium, phosphorus, potassium, selenium, and zinc. In some examples, the coagulation mineral is calcium. In some examples, the coagulation mineral is magnesium. In some examples, the coagulation minerals comprise calcium and magnesium. In some examples, the coagulation mineral (one or more) is added under conditions that promote coagulation of the pasteurized composition into a first cheese analogue product. It has been found that off-tastes may develop when adding more than 0.3 wt % or even more than 0.25 wt % or even more than 0.20 wt % of the mineral. Thus, the mineral is added in an amount of up to 0.25 wt %, or between 0.03 wt % to 0.25 wt %.

In some examples, the at least one coagulation mineral is added in an amount of up to 0.25 wt %. In some examples, the at least one coagulation mineral is added in an amount of up to 0.15 wt %. In some examples, the at least one coagulation mineral is added in an amount of up to 0.1 wt %. In some examples, the at least one coagulation mineral is added in an amount of at least 0.03 wt %. In some examples, the at least one coagulation mineral is added in an amount of at least 0.06 wt %. In some examples, the at least one coagulation mineral is added in an amount of up to 0.1 wt %. In some examples, the at least one coagulation mineral is added in an amount of up to 0.15 wt %. According to some embodiments, the coagulation mineral is added in the amount of from 0.1 to 0.35 wt %. According to some embodiments, the coagulation mineral is added in the amount of from 0.2 to 0.3 wt %. According to some embodiments, the coagulation mineral is added in the amount of from 0.15 to 0.3 wt %. According to some embodiments, the coagulation mineral is added in the amount of about 0.25 wt %.

According to some embodiments, the present invention provides a method of preparation of the cream-cheese using pre-mix composition.

According to some embodiments, the method of preparation of the cream cheese analogue comprises the steps of: 1) mixing the cream cheese analogue pre-mix according to any one of the above embodiments with water and agitating the mixture for at least 30 minutes; 2) adding plant oil and agitating under a moderate agitation; 3) heating the mixture to at least 50° C. and homogenizing; 4) pasteurizing to at least 72° C. for at least 2 minutes and cooling down; 5) optionally transferring into a fermenter under slow agitation; 6) adding calcium chloride and a bacterial culture; 7) arresting agitation; 8) fermenting the mixture upon reaching the desired pH; 9) adding NaCl under moderate agitation and10) filing into the final container.

According to some embodiments, in step 1) the water is pre-heated. According to some embodiments, the water is pre-heated up to a temperature between 35 to 45° C. According to some embodiments, the water is pre-heated up to 40° C. According to some embodiments, the agitation at step 1) is a slow agitation, e.g., 30-45 rpm. According to some embodiments, the agitation at step 1) is from 30 to 90 min, or from 30 to 60 min of from 40 to 50 min.

According to some embodiments, the agitation at step 2 is moderate agitation, e.g., 50-60 rpm. According to some embodiments, the plant oil in step 2) is replaced by a fat, e.g., plant fat. According to such embodiments, the fat is preheated until the fat is fluid or liquid.

According to some embodiments, heating at step 3) is performed using plate heat exchanger. According to some embodiments, heating at step 3) is performed to a temperature of from 50 to 70° C. of from 55 to 65° C., or to 60ºC. According to some embodiments, the homogenizing at step 3) is performed at from 40 to 300 bar, or from 50 to 250 bar. According to some embodiments, the homogenization at step 3) is performed in 2 steps. According to some embodiments, the homogenization at step 3) is performed first at from 40 to 60 bar and then at from 150 to 250 bar. According to some embodiments, the homogenization at step 3) is performed first at 50 bar and then at 200 bar.

According to some embodiments, pasteurizing at step 4) is performed to a temperature of from 72 to 90° C. for 2 to 10 minutes or from 80 to 90 for 2 to 5 minutes or at 85° C. for 2 minutes. According to some embodiments, cooling down at step 4 is to from 20 to 35° C. or to 24-32° C.

According to some embodiments, the method comprises transporting the mixture into a fermenter at step 5). According to some embodiments, the agitation at step 5) is slow agitation, e.g., 20-30 rpm.

According to some embodiments, adding calcium chloride and bacterial culture may be effected in any order. According to some embodiments, the bacterial culture is added at the final amount of 0.5 to 2 U/10 L or about 1 U/10 L. According to some embodiments, the bacterial culture is a culture of mesophilic lactic acid bacteria.

According to some embodiments, the fermentation of step 8) is performed for from 4 to 20 hours to reach the desired pH. According to other embodiments, the fermentation is performed for from 6 to 14 hours. According to some embodiments, for cream-cheese analogue III the desired pH is from 4.5 to 4.7. According to some embodiments, for cream-cheese analogue III the fermentation is performed for from 6 to 14 hours until reaching the desired pH is from 4.5 to 4.7.

Thus, according to some embodiments, the method of preparation of the cream-cheese analogue comprises the steps of: 1) mixing the cream-cheese analogue pre-mix according to any one of the above embodiments with water heated to 40° C. and slowly agitating the mixture for at least 45 minutes; 2) adding plant oil or pre-heated fluid fat and agitating under a moderate agitation; 3) heating the mixture to at least 60° C. and double-stage homogenizing at 50 and then at 200 bar; 4) pasteurizing to 85° C. for 2 minutes and cool down to 24-32° C.; 5) transferring into a fermenter under slow agitation; 6) adding calcium chloride and 1 U/10 L of Mesophilic lactic acid bacteria; 7) arresting agitation; 8) fermenting the mixture for 6-14 hours until reaching pH 4.5-4.7; 9) adding NaCl under moderate agitation; and 10) filing into the final container.

The following steps of filling the final product into the final container are relevant and incorporated in all embodiments related to preparation of the final dairy analogue food products.

According to some embodiments, filing into the final container comprises the steps of homogenizing at from 60 to 100 bar or at 70 bar e.g., to a balance tank; filling cups at room temperature and cool down rapidly; and letting the cream cheese analogue set for 24 hours until the final texture is achieved.

According to alternative embodiments, the filling the final container is a “warm” filling comprising the steps: heating to from 60 to 75° C. or to 68° C. using, e.g. tubular heat exchanger; homogenizing at from 60 to 100 bar or at 70 bar e.g. to a balance tank; filling cups at a temperature above 60° C. or above 65° C. and cool down rapidly; and let the cream cheese analogue set for 48 hours until the final texture is obtained.

According to some embodiments, the method of preparation of the cream cheese analogue comprises the steps of: 1) mixing from 8 to 12 wt % of the cream cheese analogue pre-mix comprising from 8 to 12 wt % of rBLG isolate, from 1 to 2 wt % of trisodium citrate, from 20 to 25 wt % of dextrose, from 25 to 35 of starch, with water heated to 40° C. and slowly agitating the mixture for at least 45 minutes; 2) adding from 22 to 27 wt % of plant oil such as Shia oil, and agitating under a moderate agitation; 3) heating the mixture to at least 60° C. and double-stage homogenizing at 50 and then at 200 bar; 4) pasteurizing to 85° C. for 2 minutes and cool down to 24-32° C.; 5) transferring into a fermenter under slow agitation; 6) adding about 0.6 wt % calcium chloride and 1 U/10 L of mesophilic lactic acid bacteria; 7) arresting agitation; 8) fermenting the mixture for 6-14 hours until reaching pH 4.5-4.7; 9) adding about 0.9 wt % NaCl under moderate agitation; and 10) filing into the final container.

According to any one of the above embodiments, the BLG purified from cow milk is used to prepare non-animal-free cream cheese analogue.

According to any one of the above embodiments, the present invention provides a cream-cheese analogue prepared by the methods described hereinabove. According to some embodiments, the present invention provides a cream-cheese analogue obtained or obtainable by the method according to any one of the above embodiments.

According to other embodiments, the present invention provides firm cream cheese analogues and methods for preparation thereof.

According to some embodiments, the method of preparation of the firm cream-cheese analogue comprises the steps of: 1) mixing the from 10 to 14 wt % of the firm cream cheese analogue pre-mixture e.g. comprising from 36 to 40 wt % of rBLG isolate, from 1 to 2 wt % of trisodium citrate, from 15 to 22 wt % of dextrose, from 35 to 45 of starch, and from 0.5 to 1 wt& of LBG, with water heated to 40° C. and slowly agitating the mixture for at least 45 minutes; 2) adding from 20 to 30 wt % of plant oil e.g. shea and agitating under a moderate agitation; 3) heating the mixture to at least 60° C. and double-stage homogenizing at 50 and then at 200 bar; 4) pasteurizing to 85° C. for 2 minutes and cool down to 24-32° C.; 5) transferring into a fermenter under slow agitation; 6) adding about 0.06 wt % calcium chloride and 1 U/10 L of Mesophilic lactic acid bacteria; 7) arresting agitation; 8) fermenting the mixture for 6-14 hours until reaching pH 4.5-4.7; 9) adding about 0.9 wt % NaCl under moderate agitation; and 10) filing into the final container.

Thus, according to some embodiments, the method of preparation of the high protein firm cream cheese analogue comprises the steps of: 1) mixing from 8 to 12 wt % of high-protein firm cream-cheese analogue pre-mix, e.g., comprising from 72 to 85 wt % of rBLG isolate, from 1 to 2 wt % of trisodium citrate, from 20 to 24 wt % of dextrose, and from 0.5 to 1 wt % of LBG with water heated to 40° C. and slowly agitating the mixture for at least 45 minutes; 2) adding from 20 to 24 wt % plant oil, e.g. shea oil, and agitating under a moderate agitation; 3) heating the mixture to at least 60° C. and double-stage homogenizing at 50 and then at 200 bar; 4) pasteurizing to 85° C. for 2 minutes and cool down to 24-32° C.; 5) transferring into a fermenter under slow agitation; 6) adding about 0.06 wt % calcium chloride and 1 U/10 L of Mesophilic lactic acid bacteria; 7) arresting agitation; 8) fermenting the mixture for 6-14 hours until reaching pH 4.5-4.7; 9) adding about 0.9 wt % NaCl under moderate agitation; and 10) filing into the final container.

According to some embodiments, the present invention provides a method for preparing a cream cheese analogue comprising a coagulated BLG, the method comprising: (i) dissolving from about 1 to about 8% of BLG in water; (ii) adding to the mixture of step (i) from about 0.05 to about 0.5 wt % of trisodium citrate, from about 1 to about 6 wt % of sugar, and from about 1 to about 10 wt % of a stabilizer; (iii) homogenizing the mixture of step (ii) at a temperature from about 50 to about 70° C. and at from about 100 to about 250 bar; (iv) adding to mixture of step (iii) from about 15 to about 40 wt % of an edible lipid in a liquid form, preferably a plant oil; (v) pasteurizing a mixture of step (iv) at a temperature of from about 80 to about 95° C. for from 1 to 10 min; (vi) cooling down the composition of step (v) to a temperature of from about 20 to about 40° C.; (vii) adding to the composition of step (vi) from about 0.001 to about 0.2 wt % of a coagulation mineral and Mesophilic lactic acid bacteria and allow to the fermentation until the pH reaches from 4.3 to 5; (viii) adding from about 0.6 to about 1.2 wt % of flavoring salt; and (ix) smoothing the composition of step (viii) to obtain the cream cheese analogue, wherein the mixtures in steps (i)-(vi) are substantially devoid of coagulation minerals, wherein BLG constitutes from 51 to 100 wt % of the total milk-protein content of the cream cheese analogue and wherein the cream cheese analogue comprises from 1 to 10 wt % of BLG.

According to some embodiments, the method comprises: (i) dissolving from about 1 to about 8% of BLG in water; (ii) adding to the mixture of step (i) from about 0.08 to about 0.5 wt % of trisodium citrate, from about 1 to about 6 wt % of sugar, and from about 1 to about 10 wt % of starch; (iii) homogenizing the mixture of step (ii) at a temperature from about 50 to about 70° C. and at from about 100 to about 250 bar thereby obtaining the pre-mix composition; (iv) adding to mixture of step (iii) from about 20 to about 30 wt % of an edible lipid in a liquid form, preferably a plant oil; (v) pasteurizing a mixture of step (iv) at a temperature of from about 80 to about 95° C. for from 1 to 10 min; (vi) cooling down the composition of step (v) to a temperature of from about 24 to about 40° C.; (vii) adding to the composition of step (vi) from about 0.001 to about 0.15 wt % of a coagulation mineral and Mesophilic lactic acid bacteria and allow fermentation until the pH reaches from 4.3 to 5; (viii) adding from about 0.6 to about 1.2 wt % of NaCl; and (ix) smoothing the composition of step (viii) to obtain the cream cheese analogue, wherein the mixtures in steps (i)-(vi) are substantially devoid of coagulation minerals, wherein the BLG is coagulated, and wherein the cream cheese analogue comprises from 3 to 8 wt % of BLG.

According to some embodiments, the method comprises: (i) mixing from 3 to 12% of BLG with water until the BLG is dissolved; (ii) adding to the mixture of step (i) 0.05 to 0.2 wt % of trisodium citrate, from about 1 to about 5 wt % of sugar, and from 0.05 to 0.1 wt % of a stabilizer, preferably Lcoust bean gum (LGB); (iii) adding to the mixture of step (ii) from about 15 to about 40 wt % of plant oil under agitation; (iv) homogenizing the mixture of step (iii) at a temperature from about 50 to about 70° C. and at from 100 to 250 bar; (v) pasteurizing the mixture of step (iv) at a temperature of from about 80 to about 95° C. for from 1 to 10 min; (vi) cooling down the mixture of step (v) to a temperature of from about 20 to about 40° C.; (vii) adding to the mixture of step (vi) from 0.01 to 0.15 wt % of a calcium chloride and Mesophilic lactic acid bacteria; (viii) fermenting the mixture of step (vii) until the pH reaches pH of from 4.3 to 4.9; and (ix) adding to the mixture of step (viii) from about 0.6 to 1.2 wt % of table salt to obtain a firm cream cheese analog,
wherein the mixtures of steps (i)-(vii) are devoid of coagulation minerals,
wherein the BLG is coagulated, and
wherein the firm cream cheese analogue comprises from 3 to 8 wt % of BLG.

According to some embodiments, the method comprises: (i) mixing from about 3 to about 12% of BLG with water until the BLG is dissolved; (ii) adding to the mixture of step (i) 0.05 to 0.2 wt % of trisodium citrate, from about 1 to about 5 wt % of sugar, and from 0.05 to 0.1 wt % of a stabilizer, preferably Lcoust bean gum (LGB); (iii) homogenizing the mixture of step (ii) at a temperature from about 50 to about 70° C. and at from about 100 to about 250 bar; (iv) adding to the mixture of step (iii) from about 15 to about 40 wt % of plant oil under agitation; (v) homogenizing the mixture of step (iv) at from 50 to 70° C. in from 1 to 3 steps at the pressure of from about 150 to about 300 bar; (vi) pasteurizing the mixture of step (v) at a temperature of from about 80 to about 95° C. for from about 1 to about 10 min; (vii) cooling down the mixture of step (vi) to a temperature of from about 20 to about 40° C.; (viii) adding to the mixture of step (vii) from 0.01 to 0.15 wt % of a calcium chloride and Mesophilic lactic acid bacteria; (ix) fermenting the mixture of step (viii) until the pH reaches pH of from 4.3 to 4.9; and (x) adding to the mixture of step (ix) from about 0.6 to 1.2 wt % of table salt to obtain a high protein firm cream cheese analog, wherein the mixtures of steps (i)-(vii) are devoid of coagulation minerals, and wherein the BLG is coagulated.

According to some embodiments, the present invention provides a cream cheese analogue, prepared by the method according to any one of the above embodiments. According to some embodiments, the present invention provides a cream cheese analogue, obtained or obtainable by the method according to any one of the above embodiments. According to some embodiments, the cream cheese analogue has at least one organoleptic and/or rheologic property of a corresponding dairy cream cheese selected from the group consisting of appearance, glossiness, consistency, structure, thickness, flavor, taste, and penetration strength. According to some embodiments, the cream cheese analogue has 2, 3, 4, 5, 6, 7 or 8 these properties. According to some embodiments, the cream cheese analogue has the appearance, glossiness, consistency, structure, thickness, flavor, taste, and penetration strength of a corresponding dairy cream cheese. According to some embodiments, the cream cheese analogue has 2, 3, 4, 5, 6, 7 or 8 of the following properties: Appearance: white milky colour; Glossiness: matt interior with a shiny surface; Consistency: slightly adhesive, covering the mouth but melt quickly; Structure: dense without air formation; Thickness: able to build up on a spoon; Flavour: milky, sour, cultured, acetaldehyde, alcohol, buttery (not from proteins); Taste: sour, sweet, salty; and Penetration strength: 80-100 [g].

### Cream Cheese Analogues

According to another aspect, the present invention provides a cream cheese analogue comprising from (i) from about 7 to about 20 wt % of the content of the cream cheese analogue pre-mix according to any one of the above embodiments, (ii) from about 15 to about 35 wt % of a lipid, (iii) from about 0.01 to about 0.3 wt % of calcium chloride, (iv) optionally from about 0.5 to about 1.5 wt % of flavoring salt; and (v) water up to 100 wt %, wherein the content of BLG is from 51% to 100 wt % of the total milk-protein content of the cream cheese analogue and wherein the BLG is coagulated.

All terms, embodiments and definitions disclosed in any one of the above aspects apply and are encompassed herein as well.

According to some embodiments, the cream-cheese analogue comprises: (i) from about 8 to about 12 wt % of the content of the cream cheese analogue pre-mix composition according to any one of the above embodiments; (ii) from about 20 to about 30 wt % of an edible lipid, preferably a plant oil; (iii) from about 0.6 to about 1.2 wt % of flavoring salt; (iv) from about 0.01 to about 0.15 wt % of calcium chloride; and (v) water up to 100 wt %, wherein BLG constitutes from about 51 to about 100 wt % of the total milk-protein content of the cream-cheese analogue and wherein the BLG is coagulated and the cream cheese analogue has a pH of from 4.5 to 4.7.

According to some embodiments, the cream-cheese analogue comprises: (i) from about 7 to about 15 wt % of the content of the cream cheese analogue pre-mix according to any one of the above embodiments; (ii) from about 15 to about 35 wt % of an edible lipid; (iii) from about 0.5 to about 1.5 wt % of flavoring salt; (iv) from about 0.05 to about 0.3 wt % of calcium chloride; and (v) water up to 100 wt %, wherein BLG constitutes from about 51 to about 100 wt % of the total milk-protein content of the cream cheese analogue and wherein the BLG is coagulated. According to some embodiments, the edible lipid is an oil, such as a refined coconut oil.

According to some embodiments, the cream-cheese analogue comprises: (i) from about 8 to about 20 wt % of the content of the firm cream cheese analogue pre-mix according to any one of the above embodiments; (ii) from about 20 to about 30 wt % of an edible lipid, preferably a plant oil; (iii) from about 0.6 to about 1.2 wt % of flavoring salt; (iv) from about 0.01 to about 0.3 wt % of calcium chloride; and (v) water up to 100 wt %, wherein the cream cheese analogue is a firm cream cheese analogue, the BLG constitutes from 51 to 100 wt % of the total milk-protein content of firm cream cheese analogue and the BLG is coagulated. According to some embodiments, the pH of the firm cream cheese analogue is from about 4.3 to about 4.9.

According to some embodiments, the firm cream cheese analogue comprises (i) from about 10 to about 15 wt % of the content of the firm cream-cheese pre-mix as described hereinabove; (ii) from about 20 to about 30 wt % of plant oil; (iii) from about 0.6 to about 1.2 wt % of flavoring salt such as NaCl; (iv) from about 0.01 to about 0.2 wt % of calcium chloride; (v) and water up to 100 wt %. According to some embodiments, the firm cream-cheese analogue may comprise Mesophilic lactic acid bacteria or traces or debris thereof. According to some embodiments, the firm cream cheese analogue comprises (i) about 12 wt % of the content of the cream cheese analogue pre-mix as describe hereinabove; (ii) about 25 wt % of plant oil; (iii) about 0.9 wt % of NaCl; (iv) about 0.9 wt % of calcium chloride; and (v) water up to 100 wt %. According to some embodiments, the firm cream-cheese analogue comprises (i) from 12 to 13 wt % of the content of the firm cream-cheese pre-mix; (ii) from 22 to 28 wt % of plant oil; (iii) from 0.8 to 1 wt % of NaCl; (iv) from 0.04 to 0.08 wt % of calcium chloride; and (v) water up to 100 wt %. According to some embodiments, the firm cream cheese analogue comprises about 12.4 wt % of the content of the firm cream-cheese pre-mix, about 25 wt % of plant oil, about 0.9 wt % of NaCl, about 0.06 wt % of calcium chloride and water up to 100 wt %. According to some embodiments, the plant oil is selected from shea oil, sunflower oil, coconut oil, rapeseed oil, nut oil, palm oil, kernel oil, olive oil, soya oil, cotton oil, and cocoa butter. In one embodiment, the oil is shea oil. According to some embodiments, the firm cream-cheese analogue has a pH of from 4.1 to 5.1, from 4.3 to 4.9 or from 4.5 to 4.7. According to some embodiments, the firm cream-cheese analogue has a pH of from4.5 to 4.7.

According to some embodiments, the cream-cheese analogue comprises: (i) from about 7 to about 13 wt % of the content of the cream cheese analogue pre-mix according to any one of the above embodiments; (ii) from about 20 to about 30 wt % of an edible lipid, preferably a plant oil; (iii) from about 0.6 to about 1.2 wt % of flavoring salt; (iv) from about 0.01 to about 0.15 wt % of calcium chloride; and (v) water up to 100 wt %, wherein the cream cheese analogue is a high protein firm cream cheese analogue, wherein BLG constitutes from 51 to 100 wt % of the total milk-protein content of high protein cream cheese analogue and the BLG is coagulated.

According to some embodiments, the high-protein firm cream-cheese analogue comprises (i) from about 7 to about 13 wt % of the content of the high-protein firm cream-cheese analogue pre-mix of the present invention; (ii) from about 20 to about 30 wt % of plant oil; (iii) from about 0.6 to about 1.2 wt % of flavoring salt such as NaCl (iv) from about 0.01 to about 0.2 wt % of calcium chloride; and (v) water up to 100 wt %. According to some embodiments, the firm cream-cheese analogue may comprise mesophilic lactic acid bacteria or traces or debris thereof. According to some embodiments, the high-protein firm cream-cheese analogue comprises about 12 wt % of the content of high-protein cream-cheese pre-mix; about 25 wt % of plant oil; about 0.9 wt % of NaCl; about 0.9 wt % of calcium chloride; and water up to 100 wt %. According to some embodiments, the high-protein firm cream-cheese analogue comprises from 10 to 11 wt % of the content of high-protein firm cream-cheese pre-mix; from 20 to 24 wt % of plant oil; from 0.8 to 1 wt % of NaCl; from 0.04 to 0.08 wt % of calcium chloride; and water up to 100 wt %. According to some embodiments, the plant oil is selected from shea oil, sunflower oil, coconut oil, rapeseed oil, nut oil, palm oil, kernel oil, olive oil, soya oil, cotton oil, and cocoa butter. In one embodiment, the oil is shea oil. According to some embodiments, the high-protein firm cream-cheese analogue has a pH of from 4.1 to 5.1, from 4.3 to 4.9 or from 4.5 to 4.7. According to some embodiments, the firm cream-cheese analogue has a pH of from4.5 to 4.7.

According to any one of the above embodiments, the composition may comprise BLG, rBLG or a combination thereof.

According to some embodiments, the cream cheese analogue has at least one organoleptic and/or rheologic property of a corresponding dairy cream cheese selected from appearance, glossiness, consistency, structure, thickness, flavor, taste, and penetration strength. According to some embodiments, the cream cheese analogue has 2, 3, 4, 5, 6, 7 or 8 these properties. According to some embodiments, the cream cheese analogue has the appearance, glossiness, consistency, structure, thickness, flavor, taste, and penetration strength of a corresponding dairy cream cheese.

According to some embodiments, the cream cheese analogue has 2, 3, 4, 5, 6, 7 or 8 of the following properties: Appearance: white milky colour; Glossiness: matt interior with a shiny surface; Consistency: slightly adhesive, covering the mouth but melt quickly; Structure: dense without air formation; Thickness: able to build up on a spoon; Flavour: milky, sour, cultured, acetaldehyde, alcohol, buttery (not from proteins); Taste: sour, sweet, salty and Penetration strength: 80-100 [g]. According to some embodiments, the cream cheese analogue has all of these properties.

According to any one of the above embodiments, the cream cheese analogue further comprises from 0.05 to 0.3 wt % of a preservative.

According to another aspect, the present invention provides a cream cheese analogue, comprising:
(i) from about 3 to about 10 wt % BLG;
(ii) from about 1 to about 5 wt % of a sugar;
(iii) optionally, from about 0 to about 5 wt % of a stabilizer;
(iv) from about 15 to about 35 wt % of a lipid;
(v) from about 0.01 to about 3 wt % of a coagulation mineral;
(vi) optionally, from about 0.5 to about 1.5 wt % of a flavoring salt; and
(vii) water up to 100 wt %;
wherein BLG constitutes at least 51 wt % of the total milk-protein content of the cream cheese analogue.

According to some embodiments, the present invention provides a cream cheese analogue comprising:
(i) from about 3 to about 8 wt % of BLG;
(ii) from about 0.05 to about 0.5 wt % of trisodium citrate;
(iii) from about 1 to about 6 wt % of sugar;
(iv) from about 1 to about 10 wt % of starch;
(v) from about 20 to about 30 wt % of plant oil;
(vi) from about 0.01 to about 0.1 wt % of calcium chloride;
(vii) from about 0.6 to about 1.2 wt % of flavoring salt; and
(viii) water up to 100 wt %,
wherein BLG constitutes from about 51 to about 100 wt % of the total milk-protein content of the cream cheese analogue,
wherein the BLG is coagulated, and
wherein the pH is from 4.3 to 4.9.

According to some embodiments, the present invention provides a cream cheese analogue comprising:
(i) from about 3 to about 8 wt % of BLG;
(ii) from about 0.05 to about 0.2 wt % of trisodium citrate;
(iii) from about 1 to about 5 wt % of a sugar;
(iv) from about 0.05 to about 0.1 wt % of Lcoust bean gum (LGB)
(v) from about 15 to about 40 wt % of a plant oil;
(vi) from about 0.01 to about 0.1 wt % of calcium chloride;
(vii) from about 0.6 to about 1.2 wt % of a flavoring salt; and
(viii) water up to 100 wt %,
wherein BLG constitutes from 51 to 100 wt % of the total milk-protein content of the firm cream cheese analogue,
wherein the BLG is coagulated, and
wherein the pH is from 4.3 to 4.9.

According to some embodiments, the present invention provides a cream cheese analogue comprising:
(i) from about 4 to about 8 wt % BLG;
(ii) from about 1 to about 3 wt % of a sugar;
(iii) from about 0.1 to about 4 wt % of a stabilizer;
(iv) from about 20 to about 30 wt % of a lipid;
(v) from about 0.05 to about 2.5 wt % of a coagulation mineral;
(vi) from about 0.5 to about 1.5 wt % of a flavoring salt; and
(vii) water up to 100 wt %.

According to some embodiments, the present invention provides a cream cheese analogue comprising:
(i) from about 6 to about 10 wt % of BLG;
(ii) from about 0.05 to about 0.2 wt % of trisodium citrate;
(iii) from about 1 to about 5 wt % of a sugar;
(iv) from about 0.05 to about 0.1 wt % of Lcoust bean gum (LGB)
(v) from about 15 to about 40 wt % of a plant oil;
(vi) from about 0.01 to about 0.1 wt % of calcium chloride;
(vii) from about 0.6 to about 1.2 wt % of a flavoring salt; and
(viii) water up to 100 wt %,
wherein BLG constitutes from 51 to 100 wt % of the total milk-protein content of the firm cream cheese analogue,
wherein the BLG is coagulated, and
wherein the pH is from 4.3 to 4.9.

All terms, embodiments and definitions disclosed in any one of the above aspects apply and are encompassed herein as well.

According to some embodiments, the lipid is selected from the group consisting of shea oil, sunflower oil, coconut oil, rapeseed oil, nut oil, palm oil, kernel oil, olive oil, soya oil, cotton oil, and cocoa butter (Theobroma oil), and/or the sugar is selected from the group consisting of glucose, fructose, mannose, xylose, arabinose, sucrose, dextrose, maltose, and galactose.

According to some embodiments, the present invention provides a cheese analogue comprising from 3 to 8 wt % of rBLG, from 1 to 5 wt % of a sugar, from 20 to 30 wt % of an oil, such as shea oil, from 0.1 to 0.3 wt % of calcium chloride, from 0.8 to 1 wt % of sodium chloride, and water to complete to 100 wt %. According to some embodiments, the present invention provides a cheese analogue comprising from 4 to 6 wt % of rBLG, from 1 to 3 wt % of a sugar, from 22 to 27 wt % of an oil, such as Shia oil, from 0.2 to 0.3 wt % of calcium chloride, about 0.9 wt % of sodium chloride, and water to complete to 100 wt %. According to some embodiments, the pH of the cheese analogue, is between about 5.5 and 7.5 or between 5.5 to 7 or between 5.8 and 6.7. According to some embodiments, the total solids in the cheese analogue is from 5 to 35%, or from 7 to 30% or from 10 to 28% as determined using loss-on-drying method (LOD). According to some embodiments, the protein content of the first cheese analogue is from 2 to 20 wt %, from 3 to 15 wt % or from 5 to 10 wt % as determined using e.g., Kjeldahl method with a factor conversion of 6.38. According to some embodiments, the sugar content of the cheese analogue is from 1 to 8% or from 2 to 5% as determined using HPLC under standard conditions. According to some embodiments, the salt content of the first cheese analogue is from 0.1 to 0.3% using titration AA method. According to some embodiments, the texture of the cheese analogue is from 3 to 12 N or from 4 to 10 N as determined using Texture Profile Analyzer with a spindle diameter of 11.1 mm at a speed of 100 mm/min and work depth of 50 mm. The organoleptic properties of the cheese analogue of the present invention are milky and slightly sweet by using a testing panel.

According to some embodiments, the pH of the cream cheese analogue product, is between about 4 and 5 or from 4.3 to 4.8.

According to some embodiments, the total solids in the cream cheese analogue is from about 10 to about 60%, or from about 15 to about 55% or from about 20 to about 50%, or from about 23.5 to about 45.2% as determined using loss-on-drying method (LOD).

According to some embodiments, the protein content of the cream cheese analogue is from about 1 to about 15 wt %, from about 2 to about 10 wt % or from about 3 to about 8 wt % as determined using e.g., Kjeldahl method with a factor conversion of 6.38.

According to some embodiments, the lipid content of the cream cheese analogue is from about 15 to about 40% or from about 20 to about 35% as determined using oil extraction system such as Soxhlet extraction.

According to some embodiments, sugar content of the cream cheese analogue is from 0 to 4 or from 1 to 3% as determined using HPLC under standard conditions.

According to some embodiments, salt content of the cream cheese analogue is from 0.1 to 2% or from 0.5 to 1.2% using Titration AA method.

According to some embodiments, the texture of the cream cheese analogue is from 3 to 12 N or from 4 to 20 N as determined using Texture Profile Analyzer with a spindle diameter of 11.1 mm at a speed of 100 mm/min and work depth of 50 mm.

According to any one of the above embodiments, the BLG is rBLG and the cream cheese analogue product is recombinant cream cheese analogue. According to some embodiments, the cream cheese analogue product is animal-free cream cheese analogue product.

According to some embodiments, the present invention provides a cream cheese analogue prepared by the method according to any one of the above embodiments.

According to some embodiments, the present invention provides a cream cheese analogue comprising from 3 to 8 wt % of rBLG, from 20 to 30 wt % of lipid, from 1 to 3 wt % of sugars, from 0.2 to 0.4 wt % of sodium and from 0.1 to 0.2 wt % of calcium. According to some embodiments, the pH of the cream cheese analogue product is between about 4 and 5 or from 4.3 to 4.8. According to some embodiments, the total solids in the cream cheese analogue is from 20 to 50%, or from 23.5 to 45.2% as determined using loss-on-drying method (LOD).

According to any one of the above embodiments, the cream cheese analogue further comprises from 0.05 to 0.3 wt % of a preservative.

According to some embodiments, the BLG is partly coagulated. According to some embodiments, a portion of the BLG is coagulated. According to some embodiments, the BLG is coagulated.

According to some embodiments, the cream cheese analogue has at least one organoleptic and/or rheologic property of a corresponding dairy cream cheese selected from the group consisting of appearance, glossiness, consistency, structure, thickness, flavor, taste, and penetration strength. According to some embodiments, the cream cheese analogue has 2, 3, 4, 5, 6, 7 or 8 these properties. According to some embodiments, the cream cheese analogue has the appearance, glossiness, consistency, structure, thickness, flavor, taste, and penetration strength of a corresponding dairy cream cheese. According to some embodiments, the the cream cheese analogue has 2, 3, 4, 5, 6, 7 or 8 of the following properties: Appearance: white milky colour; Glossiness: matt interior with a shiny surface; Consistency: slightly adhesive, covering the mouth but melt quickly; Structure: dense without air formation; Thickness: able to build up on a spoon; Flavour: milky, sour, cultured, acetaldehyde, alcohol, buttery (not from proteins); Taste: sour, sweet, salty; and Penetration strength: 80-100 [g].

In certain embodiments, the BLG protein: (i) confers or enhances the cheesy taste of the dairy analogue food product; (ii) enhances the aroma of the dairy analogue food product; (iii) confers or enhances the coagulation of the dairy analogue food product; (iv) enhances the Hunter lab′ “L” value of the dairy analogue food product; (v) confers or enhances the spreadability of the dairy analogue food product; (vi) confers or enhances the creaminess of the dairy analogue food product; or (vii) confers or enhances the firmness of the dairy analogue food product. According to some embodiments, the dairy analogue food product is the cream cheese analogue of the present invention.

In certain embodiments, the BLG protein: (i) confers or enhances the cheesy taste of the dairy analogue food product; (ii) enhances the aroma of the dairy analogue food product; (iii) confers or enhances the coagulation of the dairy analogue food product; (iv) enhances the Hunter lab′ “L” value of the dairy analogue food product; (v) confers or enhances the spreadability of the dairy analogue food product; (vi) confers or enhances the creaminess of the dairy analogue food product; and (vii) confers or enhances the firmness of the dairy analogue food product. According to some embodiments, the dairy analogue food product is the cream cheese analogue of the present invention.

In certain embodiments, the BLG protein: (i) confers or enhances the cheesy taste of the dairy analogue food product; (ii) confers or enhances the coagulation of the dairy analogue food product; (iii) confers or enhances the spreadability of the dairy analogue food product; (iv) confers or enhances the creaminess of the dairy analogue food product; or (v) confers or enhances the firmness of the dairy analogue food product. According to some embodiments, the dairy analogue food product is a firm cream cheese analogue. According to some embodiments, the dairy analogue food product is the high protein firm cream cheese analogue of the present invention.

In certain embodiments, the BLG protein: (i) confers or enhances the cheesy taste of the dairy analogue food product; (ii) confers or enhances the coagulation of the dairy analogue food product; (iii) confers or enhances the spreadability of the dairy analogue food product; (iv) confers or enhances the creaminess of the dairy analogue food product; and (v) confers or enhances the firmness of the dairy analogue food product. According to some embodiments, the dairy analogue food product is a firm cream cheese analogue. According to some embodiments, the dairy analogue food product is the high protein firm cream cheese analogue of the present invention.

The term “confers” as used herein relates to the BLG protein being the only ingredient of a food product which is responsible for a certain attribute or characteristic of the food product in which it is incorporated.

The term “enhances” as used herein relates to the BLG protein being one of a plurality of ingredients of a food product which is responsible for a certain attribute or characteristic of the food product in which it is incorporated.

### Methods of Preparing Ricotta Cheese Analogues

According to another aspect, provided is a method of preparing a Ricotta cheese analogue comprising a coagulated BLG, the method comprising:
(i) dissolving the cream cheese analogue pre-mix composition according to any one of the above aspects and embodiments, or the content thereof, in water and pasteurizing same;
(ii) optionally, cooling down the composition of step (i); and
(iii) adding a coagulation mineral to the composition of step (ii), if present or to the composition of step (i) if step (ii) is absent, thereby coagulating the BLG protein;
wherein BLG constitutes at least 51 wt % of the total milk-protein content of the Ricotta cheese analogue.

According to some embodiments, step (ii) is present.

According to some embodiments, the pre-mix composition comprises:
(i) from about 3 to about 8 wt % or from about 4 to about 6 wt % of BLG;
(ii) from about 1 to about 5 wt % or from about 1 to about 3 wt % of a sugar; and
(iii) from about 15 to about 35 wt % or from about 20 to about 30 wt % of an oil;
   wherein the pre-mix composition is substantially devoid of a coagulation mineral.

All terms, embodiments and definitions disclosed in any one of the above aspects apply and are encompassed herein as well.

According to some embodiments, the coagulation mineral is calcium chloride.

According to some embodiments, the method further comprises adding from about 0.6 to about 1.2 wt % of a flavoring salt.

According to some embodiments, the lipid is selected from the group consisting of shea oil, sunflower oil, coconut oil, rapeseed oil, nut oil, palm oil, kernel oil, olive oil, soya oil, cotton oil, and cocoa butter (Theobroma oil), and/or the sugar is selected from the group consisting of glucose, fructose, mannose, xylose, arabinose, sucrose, dextrose, maltose, and galactose.

According to some embodiments, the present invention provides a Ricotta cheese analogue, prepared by the methods according to any one of the above embodiments. According to some embodiments, the present invention provides a Ricotta cheese analogue, obtained or obtainable by the methods according to any one of the above embodiments.

According to some embodiments, the Ricotta analogues of the present invention has at least one property of corresponding Ricotta dairy product selected from the group consisting of appearance, glossiness, consistency, structure, thickness, taste, and flavour. According to some embodiments, the Ricotta cheese analogues of the present invention have 2, 3, 4, 5, 6, 7 or 8 properties of the dairy product. According to some embodiments, the Ricotta cheese analogues of the present invention has 2, 3, 4, 5, 6, 7 or 8 of the following properties: Appearance: white milky colour; Glossiness: matt interior with a shiny surface; Consistency: slightly adhesive, covering the mouth but melt quickly; Structure: dense without air formation; Thickness: able to build up on a spoon; Flavour: mild milky, buttery (not from protein); and Taste: Sweet, Salty, metallic. According to some embodiments, the Ricotta cheese analogues of the present invention has all these properties.

### Ricotta Cheese Analogue

According to yet another aspect, the present invention provides a Ricotta cheese analogue, comprising:
(i) from about 3 to about 8 wt % BLG;
(ii) from about 1 to about 5 wt % of a sugar;
(iii) from about 15 to about 35 wt % of a lipid; and
(iv) water up to 100 wt %;
wherein BLG constitutes at least 51 wt % of the total milk-protein content of the Ricotta cheese analogue.

All terms, embodiments and definitions disclosed in any one of the above aspects apply and are encompassed herein as well.

According to some embodiments, the lipid is selected from the group consisting of shea oil, sunflower oil, coconut oil, rapeseed oil, nut oil, palm oil, kernel oil, olive oil, soya oil, cotton oil, and cocoa butter (Theobroma oil), and/or the sugar is selected from the group consisting of glucose, fructose, mannose, xylose, arabinose, sucrose, dextrose, maltose, and galactose.

According to some embodiments, the BLG is coagulated.

According to some embodiments, the pH of the Ricotta cheese analogue is between about 5.5 and 7.5 or between 5.5 to 7 or between 5.8 and 6.7. According to some embodiments, the total solids in the Ricotta cheese analogue is from 5 to 35, or from 7 to 30% or from 10 to 28% as determined using loss-on-drying method (LOD).

According to some embodiments, the protein content of the Ricotta cheese analogue is from 2 to 20 wt %, from 3 to 15 wt % or from 5 to 10 wt % as determined using e.g., Kjeldahl method with a factor conversion of 6.38.

According to some embodiments, sugar content of Ricotta cheese analogue is from 1 to 8% or from 2 to 5% as determined using HPLC under standard conditions.

According to some embodiments, salt content of the Ricotta cheese analogue is from 0.1 to 0.3% using Titration AA method.

According to some embodiments, the texture of the Ricotta cheese analogue is from 3 to 12 N or from 4 to 10 N as determined using Texture Profile Analyzer with a spindle diameter of 11.1 mm at a speed of 100 mm/min and work depth of 50 mm.

According to any one of the above embodiments, the BLG is rBLG and the cheese analogue product is recombinant cheese analogue. According to some embodiments, the Ricotta cheese analogue product is animal-free cheese analogue product.

According to some embodiments, the Ricotta analogues of the present invention has at least one property of corresponding Ricotta dairy product selected from the group consisting of appearance, glossiness, consistency, structure, thickness, taste, and flavour. According to some embodiments, the Ricotta cheese analogues of the present invention have 2, 3, 4, 5, 6, 7 or 8 properties of the dairy product. According to some embodiments, the Ricotta cheese analogues of the present invention has 2, 3, 4, 5, 6, or 7 of the following properties: Appearance: white milky colour; Glossiness: matt interior with a shiny surface; Consistency: slightly adhesive, covering the mouth but melt quickly; Structure: dense without air formation; Thickness: able to build up on a spoon; Flavour: mild milky, buttery (not from protein); and Taste: Sweet, Salty, metallic. According to some embodiments, the Ricotta cheese analogues of the present invention has all these properties.

The present invention provides yogurt analogues and methods of producing same.

### Yogurt Analogue Pre-Mix Compositions

According to another aspect, the present invention provides a yogurt analogue pre-mix composition, comprising:
(i) from about 50 to about 60 wt % BLG;
(ii) optionally, from about 0.8 to about 2 wt % of a coagulation mineral chelator;
(iii) from about 30 to about 50 wt % of a sugar; and
(iv) from about 0.5 to about 3 wt % of a stabilizer;
wherein the pre-mix composition is substantially devoid of coagulation minerals, and
wherein BLG constitutes at least 51 wt % of the total milk-protein content of the pre-mix composition.

All terms, embodiments and definitions disclosed in any one of the above aspects apply and are encompassed herein as well.

According to some embodiments, the yogurt analogue pre-mix composition comprises from about 0.8 to about 2 wt % of a coagulation mineral chelator.

According to some embodiments, the stabilizer is a pectin.

According to some embodiments, the yogurt analogue pre-mix composition comprises from about 50 to about 60 wt %, from about 52 to about 58 wt % or from about 54 to about 56 wt % of BLG. According to some embodiments, the yogurt analogue pre-mix composition comprises from about 35 to about 45 wt % of a sugar. According to some embodiments, the yogurt analogue pre-mix composition comprises from about 1 to about 3 wt %, or from about 1.5 to about 2.5 wt % of a stabilizer. According to some embodiments, the BLG is rBLG. According to some embodiments, the rBLG is the rBLG of the present invention.

According to some embodiments, the present invention provides a yogurt analog pre-mix composition comprising from 48 to 62 wt %, from 50 to 60 wt % of rBLG, from 0.8 to 2 wt % of trisodium citrate, from 30 to 50 wt % of sugar, and from 0.5 to 3 wt % of pectin as a stabilizer. According to some embodiments, the yogurt analogue pre-mix composition comprising from 52 to 57 wt % of rBLG, from 1.3 to 1.8 wt % of trisodium citrate, from 35 to 45 wt % of sugar, and from 1.3 to 1.8 wt % of pectin. According to some embodiments, the sugar is dextrose. According to some embodiments, the yogurt analogue pre-mix composition comprises from 54 to 56 wt % of the rBLG. According to some embodiments, the yogurt analogue pre-mix composition comprises from 40 to 50 wt % or from 42 to 48 wt % of the rBLG protein. According to some embodiments, the yogurt analogue pre-mix composition comprises from 1.3 to 1.6 wt % of trisodium citrate. According to some embodiments, the sugar is dextrose. According to other embodiments, the yogurt pre-mix composition comprises from 38 to 43 wt % of sugar such as dextrose. According to other embodiments, the yogurt analogue pre-mix composition comprises from 1.4 to 1.7 wt % of pectin. According to some embodiments, the yogurt analogue pre-mix composition comprising about 55% of rBLG isolate comprising from about 85 to about 99 wt % of rBLG, about 1.5 wt % of trisodium citrate, about 41 wt % of dextrose, and about 1.6 wt % of pectin. According to some embodiment, BLG purified from milk is used instead of rBLG.

### Methods of Preparing Yogurt Analogues

According to another aspect, the present invention provides a method of preparing a yogurt analogue comprising a coagulated BLG, the method comprising:
(i) mixing (a) water, (b) from about 5 to about 20 wt % of the yogurt analogue pre-mix according to any one of the above embodiments and aspect or the content thereof, and (c) from about 1 to about 8 wt % of a lipid;
(ii) pasteurizing the composition of step (i);
(iii) optionally, cooling down the composition of step (ii);
(iv) adding a coagulation mineral and acidifying the composition of step (ii), if step (iii) is absent, or of step (iii) to reach pH of from about 3.5 to about 4.8, thereby coagulating the BLG protein; and
(v) adding from about 5 to about 15 wt % of a sugar,
wherein BLG constitutes at least 51 wt % of the total milk-protein content of the yogurt analogue. According to some embodiments, the method comprises step (ii).

All terms, embodiments and definitions disclosed in any one of the above aspects apply and are encompassed herein as well.

According to some embodiments, the acidification in step (iv) is performed by adding lactic acid bacteria and allowing fermenting until the pH reaches the values of from about 3.5 to about 4.8.

According to some embodiments, the present invention provides a method for preparation of a yogurt analogue comprising a coagulated BLG, the method comprising:
(i) adding to a water solution comprising from 5 to 20 wt % of a yogurt pre-mix composition according to any one of the above embodiments, from 1 to 8 wt % of an edible lipid in a liquid form, preferably a plant oil;
(ii) pasteurizing the mixture of step (i) at a temperature of from about 80 to about 95° C. for from 1 to 10 min;
(iii) cooling down the composition of step (ii) to a temperature of from about 30 to about 45° C.;
(iv) adding to the composition of step (iii) from about 0.01 to about 0.15 wt % of a coagulation mineral and Thermophilic lactic acid bacteria and allow fermenting until the pH reaches from 3.5 to 4.8; and
(v) adding to the composition of step (iv) from about 5 to about 15 wt % of sucrose under moderate agitation,
wherein BLG constitutes from 51 to 100 wt % of the protein content of the yogurt analogue,
wherein the yogurt analogue comprises from 3 to 8 wt % of BLG, and
   wherein the BLG is coagulated. Adding the pre-mix has the meaning of adding the ingredients of the pre-mix composition.

According to some embodiments, the method of preparation of the yogurt analogue comprises the steps of:
1) mixing from 8 to 12 wt % of yogurt analogue pre-mixture e.g., comprising from 52 to 57 wt % of BLG, from 1 to 2 wt % of trisodium citrate, from 35 to 45 wt % of dextrose, and from 1 to 2 wt % of pectin, with water heated to 40° C. and slowly agitating the mixture for at least 45 minutes;
2) adding from about 1 to about 3 wt % plant oil or a preheated fat and agitating under a moderate agitation;
3) heating the mixture to at least 60° C. and double-stage homogenizing at 50 and then at 200 bar;
4) pasteurizing to 85° C. for 2 minutes and cool down to 37-45° C.;
5) transferring into a fermenter under slow agitation;
6) adding about 0.07 wt % calcium chloride and 1 U/10 L of thermophilic lactic acid bacteria;
7) arresting agitation;
8) fermenting the mixture for 5 to 10 hours until reaching pH 3.9-4.4;
9) adding about 8 wt % sucrose under moderate agitation; and 10) optionally filing into the final container.

According to some embodiments, the filling comprises:
Mixing under moderate agitation e.g., in a balance tank;
Filling at room temperature and cool down rapidly; and
Letting the yogurt analogue set for 24 hours until the final texture is achieved.

According to another embodiments, the present invention provides a method for preparation of a yogurt analogue comprising a coagulated BLG, the method comprising:
(i) dissolving from about 2 to about 8% of BLG in water;
(ii) adding to the solution of step (i) from about 0.08 to about 0.5 wt % of trisodium citrate, from about 2 to about 15 wt % of sugar, and from about 0.5 to about 1 wt % of a stabilizer, such as pectin;
(iii) homogenizing the mixture of step (ii) at a temperature from about 50 to about 70° C. and at the pressure of from about 100 to about 250 bar;
(iv) adding to mixture of step (iii) from about 1 to about 8 wt % of a plant oil;
(v) pasteurizing a mixture of step (iv) at a temperature of from about 80 to about 95° C. for from about 1 to about 10 min;
(vi) cooling down the composition of step (v) to a temperature of from about 30 to about 45° C.;
(vii) adding to the composition of step (vi) from about 0.001 to 0.15 wt % of a coagulation mineral and Thermophilic lactic acid bacteria and allow fermentation until the pH reaches from 3.5 to 4.8; and
(viii) adding to the composition of step (vii) from about 5 to about 15 wt % of sucrose under moderate agitation.
wherein the mixtures in steps (i)-(vi) are devoid of coagulation minerals,
wherein BLG constitutes from 51 to 100 wt % of the yogurt analogue,
wherein the yogurt analogue comprises from 3 to 8 wt % of BLG, and
wherein the BLG is coagulated.

According to some embodiments, the present invention provides a yogurt analogue prepared by the method according to any one of the above embodiments. According to some embodiments, the present invention provides a yogurt analogue obtained or obtainable by the method according to any one of the above embodiments.

### Yogurt Analogues

According to some embodiments, the present invention provides a yogurt analogue comprising: (i) from 7 to 13 wt % of the content of the yogurt pre-mix formulation according to any one of the above embodiments; (ii) from 1 to 6 wt % of plant oil; (iii) from 0.01 to 0.15 wt % of calcium chloride; (iv) from 4 to 12 wt % of sugar; and (v) water up to 100 wt %, wherein the content of BLG is from 51 to 100 wt % of the protein content of the yogurt analogue and wherein the yogurt analogue comprises from 3 to 8 wt % of BLG and the BLG is coagulated.

According to some embodiments, the yogurt analogue comprises (i) from 7 to 13 wt % of the content of the yogurt analogue pre-mix composition of the present invention; (ii) from 1 to 6 wt % of plant oil; (iii) from 0.01 to 1 wt % of calcium chloride; (iv) from 4 to 12 wt % of sugar, such as sucrose; and (v) water up to 100 wt %. According to some embodiments, the yogurt analogue may comprise thermophilic lactic acid bacteria or traces or debris thereof. According to some embodiments, the yogurt analogue comprises about 9.5 wt % of the content of the yogurt analogue pre-mix; about 3 wt % of plant oil; about 0.07 wt % of calcium chloride; about 8 wt % of sucrose; and water up to 100 wt %. According to some embodiments, the yogurt analogue comprises from 9 to 10 wt % of the content of the yogurt analogue pre-mix; from 2 to 4 wt % of plant oil; from 0.06 to 0.08 wt % of calcium chloride; from 7 to 9 wt % of sucrose; and water up to 100 wt %. According to some embodiments, the plant oil is selected from shea oil, sunflower oil, coconut oil, rapeseed oil, nut oil, palm oil, kernel oil, olive oil, soya oil, cotton oil, and cocoa butter. In one embodiment, the oil is shea oil. According to some embodiments, the yogurt analogue has a pH of from 3.5 to 4.8, from 3.7 to 4.6 or from 3.9 to 4.4. According to some embodiments, the firm yogurt analogue has a pH of from 3.9 to 4.4.

According to any one of the above embodiments, the rBLG or some of it may be replaced by a BLG isolated from cow milk.

According to another aspect, the present invention provides a yogurt analogue, comprising:
(i) from about 2 to about 8 wt % BLG;
(ii) optionally, from about 0.08 to about 0.5 wt % of a coagulation mineral chelator;
(iii) from about 2 to about 15 wt % of a sugar;
(iv) from about 0.05 to about 0.3 wt % of a stabilizer;
(v) from about 1 to about 6 wt % of a lipid;
(vi) from about 0.01 to about 0.15 wt % of a coagulation mineral; and
(vii) water up to 100 wt %,
   wherein BLG constitutes at least 51 wt % of the total milk-protein content of the yogurt analog. According to some embodiments, the yogurt analogue comprises from about 0.08 to about 0.5 wt % of a coagulation mineral chelator.

According to some embodiments, the yogurt analog comprises:
(i) from 2 to 8 wt % of BLG;
(ii) from 0.08 to 0.5 wt % of trisodium citrate;
(iii) from 2 to 15 wt % of sugar;
(iv) from 00.5 to 1 wt % of a stabilizer, preferably Pectin
(v) from 1 to 6 wt % of plant oil;
(vi) from 0.01 to 0.15 wt % of calcium chloride;
(vii) from 4 to 12 wt % of sugar; and
(viii) water up to 100 wt %,
wherein the content of BLG is from 51 to 100 wt % of the protein content of the yogurt analog,
wherein the BLG is coagulated, and
wherein the yogurt analog has pH of from about 3.7 to about 4.6

All terms, embodiments and definitions disclosed in any one of the above aspects apply and are encompassed herein as well.

According to some embodiments, BLG constitutes at least 51 wt %, at least 53 wt %, or at least 55 wt % of the total milk-protein content of the yogurt analogue. According to some embodiments, BLG constitutes at least 60 wt %, at least 65 wt %, at least 70 wt %, at least 75 wt %, at least 80 wt %, at least 85 wt %, at least 90%, at least 95 wt %, at least 97 wt %, at least 98 wt % or at least 99 wt % of the total milk-protein content of the yogurt analogue. According to some embodiments, the BLG constitutes from about 51 wt % to about 100 wt %, from about 55 wt % to about 95 wt %, from 60 wt % to about 90 wt %, from 65 wt % to about 85 wt %, from 70 wt % to about 80 wt %, from 70 wt % to about 99 wt %, from 75 wt % to about 95 wt %, from 85 wt % to about 95 wt %, from 90 wt % to about 100 wt % or from 95 wt % to about 99 wt % of the total milk-protein content of the yogurt analogue. According to some embodiments, the BLG is the sole milk protein in yogurt analogue. According to any one of the above embodiments, BLG constitutes at least 51 wt %, at least 53 wt %, at least 55 wt %, at least 60 wt %, at least 65 wt %, at least 70 wt %, at least 75 wt %, at least 80 wt %, at least 85 wt %, at least 90 wt %, at least 95 wt %, at least 97 wt %, at least 98% or at least 99 wt % of the total protein content of the yogurt analogue. According to some embodiments, BLG constitutes from about 51 wt % to about 100 wt %, from about 55 wt % to about 99 wt %, from about 60 wt % to about 98 wt %, from about 65 wt % to about 97 wt %, from about 70% to about 95 wt %, from about 75 wt % to about 93 wt %, from about 80 wt % to about 90 wt %, from about 60 wt % to about 80 wt %, from about 55 wt % to about 95 wt %, from 60 wt % to about 90 wt %, from about 65 wt % to about 85 wt %, from about 70 wt % to about 80 wt %, from 70 wt % to about 99 wt %, from 75 wt % to about 95 wt %, from 85 wt % to about 95 wt %, from 90 wt % to about 100 wt % or from 95 wt % to about 99 wt %, or about 100 wt % of the total protein content of the yogurt analogue. According to some embodiments, the BLG is rBLG. According to some embodiments, the BLG is the rBLG of the present invention. According to any one of the above embodiments, the yogurt analogue comprises coagulated BLG. According to some embodiments, from about 40 to about 100%, from about 45 to about 99%, from about 50% to about 95%, from about 55% to about 95%, from about 60% to about 94%, from about 65% to about 93%, from about 70% to about 92%, from about 75% to about 90%, from about 80% to about 90%, from about 80% to about 95%, from about 85% to about 95% or from about 85 to about 99% of the BLG is coagulated.

According to some embodiments, the yogurt analogue has at least one organoleptic and/or rheologic property of a corresponding dairy yogurt selected from the group consisting of appearance, glossiness, consistency, structure, thickness, flavor, and taste. According to some embodiments, the yogurt analogue has 2, 3, 4, 5, 6, or 7 these properties. According to some embodiments, the yogurt analogue has the appearance, glossiness, consistency, structure, thickness, flavor, and taste, of a corresponding dairy yogurt. According to some embodiments, the yogurt analogue has 2, 3, 4, 5, 6, or 7 of the following properties: Appearance: white milky colour; Glossiness: matt interior with a shiny surface; Consistency: slightly adhesive, covering the mouth but melt quickly; Structure: dense without air formation; Thickness: able to build up on a spoon; Flavour: milky, sour, cultured, acetaldehyde, alcohol, buttery (not from proteins); and Taste: sour, sweet, salty. According to some embodiments, the yogurt analogue has all these properties.

In certain embodiments, the BLG protein: (i) enhances the aroma of the dairy analogue food product; (ii) enhances the Hunter lab′ “L” value of the dairy analogue food product; (iii) confers or enhances the coagulation of the dairy analogue food product; (iv) confers or enhances the spreadability of the dairy analogue food product; or (v) enhances the creaminess of the dairy analogue food product. According to some embodiments, the dairy analogue food product is a yogurt analogue of the present invention.

In certain embodiments, the BLG protein: (i) enhances the aroma of the dairy analogue food product; (ii) enhances the Hunter lab′ “L” value of the dairy analogue food product; (iii) confers or enhances the coagulation of the dairy analogue food product; (iv) confers or enhances the spreadability of the dairy analogue food product; and (v) enhances the creaminess of the dairy analogue food product. According to some embodiments, the dairy analogue food product is a yogurt analogue of the present invention.

The present invention provides ice cream analogues and methods for producing same.

### Ice Cream Analogue Pre-Mix Compositions

According to another aspect, the present invention provides an ice cream analogue pre-mix composition, comprising:
(i) from about 6 to about 14 wt % BLG;
(ii) from about 80 to about 85 wt % of a sugar;
(iii) optionally, from about 0.2 to about 0.4 wt % of a coagulation mineral;
(iv) from about 5 to about 7 wt % of dietary fibers;
(v) from about 0.5 to about 1.5 wt % of an emulsifier; and
(vi) from about 0.3 to about 1 wt % of a stabilizer;
wherein BLG constitutes at least 51 wt % of the total milk-protein content of the pre-mix composition.

All terms, embodiments and definitions disclosed in any one of the above aspects apply and are encompassed herein as well.

According to some embodiment, the ice cream analogue pre-mix composition comprises from 7 to 9 wt % of BLG or about 8 wt % of BLG. According to some embodiments, the ice cream analogue pre-mix composition comprises from 6 to 8 wt % of BLG protein. According to some embodiments, the BLG is rBLG.

According to some embodiments, the ice cream analogue pre-mix composition comprises from 81 to 84 wt % or about 83 wt % of sugars. According to some embodiments, the sugars comprise dextrose, sucrose, glucose, glucose syrup or a combination thereof. According to some embodiments, the ice cream analogue pre-mix composition comprises from 18 to 22 wt % of dextrose, from 40 to 43 wt % of sucrose, and from 18 to 22 wt % of 80/38 glucose syrup.

According to some embodiments, the emulsifier is sucrose ester SP70. According to some embodiments, the emulsifier is a non-ionic surfactant sucrose stearate.

According to some embodiments, the ice cream analogue pre-mix composition comprises from about 0.2 to about 0.4 wt % or from 0.3 to 0.5 wt % of coagulation minerals.

According to some embodiments, the coagulation mineral is calcium chloride.

According to some embodiments, the dietary fibers comprise inulin.

According to some embodiments, the stabilizer is selected from the group consisting of LBG, carrageenan and a combination thereof. According to some embodiments, the ice cream analogue pre-mix composition comprises from 0.4 to 0.6 wt % of LBG and from 0.1 to 0.2 wt % of Carrageenan.

According to some embodiments, the ice cream analogue pre-mix composition further comprises cream flavor such as Edlong #1413271. According to some embodiments, the ice cream analogue pre-mix composition comprises from 0.8 to 0.9 wt % of the cream flavor.

According to some embodiments, the ice cream analogue pre-mix composition further comprises color agent such as Vanilla yellow. According to some embodiments, the ice-cream premix composition comprises from 0.005 to 0.02 wt % of the color agent.

According to some embodiments, the ice-cream premix composition comprises from 5 to 7 wt % of inulin, from 6 to 10 wt % of rBLG, from 18 to 22 wt % of dextrose, from 40 to 43 wt % of sucrose, from 18 to 22 wt % of glucose syrup, from 0.5 to 1.5 wt % of sucrose ester SP70, from 0.3 to 0.4 wt % of calcium chloride, from 0.4 to 0.6 wt % of LBG, from 0.1 to 0.2 wt % of Carrageenan and optionally from 0.8 to 0.9 wt % of cream flavor and optionally about 0.01 wt % of color agent.

### Methods of Preparing Ice Cream Analogues

According to another aspect, the present invention provides a method of preparing an ice cream analogue, comprising:
(i) mixing (a) water, (b) from about 20 to about 30 wt % of the ice cream premix composition according to any one of the above aspects and embodiments, or the content thereof, and (c) from about 7 to about 12 wt % of a lipid;
(ii) homogenizing the composition of step (i); and
(iii) pasteurizing the composition of step (ii);
wherein BLG constitutes at least 51 wt % of the total milk-protein content of the ice cream analogue.

All terms, embodiments and definitions disclosed in any one of the above aspects apply and are encompassed herein as well.

According to some embodiments, the present invention provides a method of preparation of the ice cream analogue, the method comprises the steps of:
1) mixing from about 20 to about 30 wt % ice cream analogue pre-mix composition according to any one of the above embodiments with water heated to 40° C. and slowly agitating the mixture for at least 30 or at least 45 minutes;
2) adding plant oil and agitating under a moderate agitation;
3) heating the mixture to at least 60° C. and homogenizing at least once or twice;
4) pasteurizing to 72 or to 85° C. for 2 minutes and cool down to from 8 to 2° C.;
5) letting the mixture to mature for 2 to 6 or 3 to 5 hours, e.g., in a balance tank;
6) entering the mix into the ice cream maker and producing ice cream; and
7) storing at −18° C.

According to some embodiments, the present invention provides a method of preparation of the ice cream analogue, the method comprises the steps of:
1) mixing from about 20 to about 35 wt % of the ice cream analogue pre-mix composition according to any one of the above embodiments with water heated to 40° C. and slowly agitating the mixture for at least 30 or at least 45 minutes;
2) adding from about 7 to about 12 wt % of plant oil and agitating under a moderate agitation;
3) heating the mixture to at least 60° C. and homogenizing once or twice;
4) pasteurizing to 72 or to 85° C. for 2 minutes and cool down to 4° C.;
5) allowing the mixture to mature for 2 to 6 or 3 to 5 hours, e.g., in a balance tank;
6) entering the mix into the ice cream maker and produce ice cream; and
7) storing at −18° C.

According to some embodiments, the homogenization of step 3) is a double-stage homogenization, e.g., at 50 and then at 200 bar.

According to some embodiments, the present invention provides an ice cream analogue, prepared by the method according to any one of the above embodiments. According to some embodiments, the present invention provides an ice cream analogue, obtained or obtainable by the method according to any one of the above embodiments.

According to some embodiments, the present invention provides an ice cream analogue comprising the content of the ice cream analogue pre-mix composition of the present invention. According to some embodiments, the ice cream analogue comprises (i) from 20 to 35 wt % of the content of the ice cream analogue pre-mix composition; (ii) from 5 to 15 wt % of plant oil; (iii) optionally from 0.05 to 0.2 wt % of vanilla extract; and (iv) water up to 100 wt %. According to some embodiments, the ice cream analogue comprises from 27 to 32 wt % of the content of the ice cream analogue pre-mix composition. According to some embodiments, the ice cream analogue comprises from 7 to 12 wt % of plant oil. According to some embodiments, the plant oil is selected from shea oil, sunflower oil, coconut oil, rapeseed oil, nut oil, palm oil, kernel oil, olive oil, soya oil, cotton oil, and cocoa butter. In one embodiment, the oil is shea oil.

According to some embodiments, the ice cream analogue comprises from 27 to 32 wt % of the content of the ice cream analogue pre-mix composition; from 7 to 12 wt % of plant oil; optionally from 0.05 to 0.15 wt % of vanilla extract; and water up to 100 wt %. According to some embodiments, the ice cream analogue comprises about 29 wt % of the content of the ice cream analogue pre-mix composition; about 10 wt % of plant oil; about 0.1 wt % of vanilla extract; and water up to 100 wt %.

In some embodiments, a BLG purified from cow milk is used instead of rBLG to prepare the ice cream analog.

### Ice Cream Analogues

According to another aspect, the present invention provides an ice cream analogue, comprising:
(i) from about 1 to about 4 wt % BLG;
(ii) from about 10 to about 40 wt % of a sugar;
(iii) optionally, from about 0.05 to about 0.15 wt % of a coagulation mineral;
(iv) from about 1.5 to about 2.1 wt % of dietary fibers;
(v) from about 0.15 to about 0.45 wt % of an emulsifier;
(vi) from about 0.1 to about 0.3 wt % of a stabilizer;
(vii) from about 5 to about 15 wt % of a lipid;
(viii) optionally, from about 0.05 to about 0.2 wt % of Vanilla extract; and
(ix) water up to 100 wt %;
wherein BLG constitutes at least 51 wt % of the total milk-protein content of the ice cream analogue.

All terms, embodiments and definitions disclosed in any one of the above aspects apply and are encompassed herein as well. According to some embodiments, the ice cream analogue comprises from about 0.05 to about 0.15 wt % of a coagulation mineral.

According to some embodiments, the ice cream analogue of the present invention has at least one organoleptic and/or rheologic property of a corresponding dairy ice cream selected from the group consisting of appearance, glossiness, consistency, structure, thickness, flavor, taste, and overrun. According to some embodiments, the ice cream analogue has 2, 3, 4, 5, 6, 7 or 8 these properties. According to some embodiments, the ice cream analogue has appearance, glossiness, consistency, structure, thickness, flavor, taste, and overrun of a corresponding dairy ice cream. According to some embodiments, the ice cream analogue has 2, 3, 4, 5, 6, 7 or 8 of the following properties: Appearance: white milky color; Glossiness: shiny surface and interior; Consistency: slightly adhesive, covering the mouth but melt quickly; Structure: Airy but stable at freezing temperature; Thickness: able to build up on a spoon; Flavor: Milky, buttery, and vanilla (not from protein); Taste: Sweet; Overrun: 30-40%. According to some embodiments, the ice cream analogue has all these properties.

According to some embodiments, the BLG may constitute from 51 to 100 wt % of the total milk-protein content of the ice cream analogue. According to some embodiments, BLG constitutes at least 53 wt % of the total milk-protein content of the ice cream analogue. According to some embodiments, BLG constitutes at least 55 wt % of the total milk-protein content of the ice cream analogue. According to some embodiments, BLG constitutes at least 60 wt %, at least 65 wt %, at least 70 wt %, at least 75 wt %, at least 80 wt %, at least 85, at least 90 wt %, at least 95 wt %, at least 97 wt %, at least 98 wt % or at least 99 wt % of the total milk-protein content of the ice cream analogue. According to any one of the above embodiments, the BLG constitutes from about 51 wt % to about 100 wt %, from about 55 wt % to about 95 wt %, from 60 wt % to about 90 wt %, from 65 wt % to about 85 wt %, from 70 wt % to about 80 wt %, from 70 wt % to about 99 wt %, from 75 wt % to about 95 wt %, from 85 wt % to about 95 wt %, from 90 wt % to about 100 wt % or from 95 wt % to about 99 wt % of the total milk-protein content of the ice cream analogue. According to some embodiments, the BLG is the sole milk protein in the ice cream analogue. According to any one of the above embodiments, BLG constitutes at least 51 wt %, at least 53 wt %, at least 55 wt %, at least 60 wt %, at least 65 wt %, at least 70 wt %, at least 75 wt %, at least 80 wt %, at least 85 wt %, at least 90 wt %, at least 95 wt %, at least 97 wt %, at least 98% or at least 99 wt % of the total protein content of the ice cream analogue. According to some embodiments, BLG constitutes from about 51 wt % to about 100 wt %, from about 55 wt % to about 99 wt %, from about 60 wt % to about 98 wt %, from about 65 wt % to about 97 wt %, from about 70% to about 95 wt %, from about 75 wt % to about 93 wt %, from about 80 wt % to about 90 wt %, from about 60 wt % to about 80 wt %, from about 55 wt % to about 95 wt %, from 60 wt % to about 90 wt %, from about 65 wt % to about 85 wt %, from about 70 wt % to about 80 wt %, from 70 wt % to about 99 wt %, from 75 wt % to about 95 wt %, from 85 wt % to about 95 wt %, from 90 wt % to about 100 wt % or from 95 wt % to about 99 wt %, or about 100 wt % of the total protein content of the ice cream analogue.

In certain embodiments, the BLG protein: (i) confers or enhances the milky taste of the dairy analogue food product; (ii) enhances the density of the dairy analogue food product; (iii) confers or enhances the creamy mouthfeel of the dairy analogue food product; (iv) decreases the overrun of the dairy analogue food product; (v) decreases the size of the ice crystals of the dairy analogue food product; or (vi) enhances the firmness of the dairy analogue food product. According to some embodiments, the dairy analogue food product is an ice cream analogue.

In certain embodiments, the BLG protein: (i) confers or enhances the milky taste of the dairy analogue food product; (ii) enhances the density of the dairy analogue food product; (iii) confers or enhances the creamy mouthfeel of the dairy analogue food product; (iv) decreases the overrun of the dairy analogue food product; (v) decreases the size of the ice crystals of the dairy analogue food product; and (vi) enhances the firmness of the dairy analogue food product. According to some embodiments, the dairy analogue food product is an ice cream analogue.

According to any one of the above aspects and embodiments, the BLG may constitute from 51 to 100 wt % of the total milk-protein content of the dairy analogue food product or pre-mix composition. According to any one of the above embodiments and aspects, BLG constitutes at least 53 wt % of the total milk-protein content of the dairy analogue food product pre-mix composition. According to some embodiments, BLG constitutes at least 55 wt % of the total milk-protein content of the dairy analogue food product pre-mix composition. According to some embodiments, BLG constitutes at least 60 wt %, at least 65 wt %, at least 70 wt %, at least 75 wt %, at least 80 wt %, at least 85, at least 90 wt %, at least 95 wt %, at least 97 wt %, at least 98 wt % or at least 99 wt % of the total milk-protein content of the dairy analogue food product pre-mix composition. According to some embodiments, the BLG is the sole milk protein in the final dairy analogue food product pre-mix composition. According to any one of the above embodiments, BLG constitutes at least 51 wt %, at least 53 wt %, at least 55 wt %, at least 60 wt %, at least 65 wt %, at least 70 wt %, at least 75 wt %, at least 80 wt %, at least 85 wt %, at least 90 wt %, at least 95 wt %, at least 97 wt %, at least 98% or at least 99 wt % of the total protein content of the final dairy analogue food product pre-mix composition. According to some embodiments, BLG constitutes from about 51 wt % to about 100 wt %, from about 55 wt % to about 99 wt %, from about 60 wt % to about 98 wt %, from about 65 wt % to about 97 wt %, from about 70% to about 95 wt %, from about 75 wt % to about 93 wt %, from about 80 wt % to about 90 wt %, from about 60 wt % to about 80 wt %, from about 55 wt % to about 95 wt %, from 60 wt % to about 90 wt %, from about 65 wt % to about 85 wt %, from about 70 wt % to about 80 wt %, from 70 wt % to about 99 wt %, from 75 wt % to about 95 wt %, from 85 wt % to about 95 wt %, from 90 wt % to about 100 wt % or from 95 wt % to about 99 wt %, or about 100 wt % of the total protein content of the final dairy analogue food product pre-mix composition. According to some embodiments, BLG may be is a sole protein in the dairy analogue food product pre-mix composition. According to some embodiments, the BLG is a recombinant BLG. According to some embodiments, the rBLG is the rBLG of the present invention. According to some embodiments, the rBLG is the rBLG prepared by the methods of the present invention.

According to any one of the above aspects and embodiments, the dairy analogue food product or a pre-mix composition may comprise a coagulation mineral chelator, e.g., from about 0.08 to about 0.5 wt % of a coagulation mineral chelator.

According to any one of the embodiments and aspects, the composition or the cheese analogue which was acidified via lactic bacteria acidification comprises traces of the bacteria.

According to any one of the methods described above, the method further comprises filling the product into a container. Non-limiting examples of the process are described in Example 4.

A non-limiting list of cheese analogue products include Ricotta analogue, cream cheese analogue, labneh analogue, and Greek yogurt analogue. According to some embodiments, the content and the composition of a dairy analog food product or dairy analog food product pre-mix is as defined in any one of Tables 3, 4, 6, 9, 11, 12, 14, 15, 16, 19, 20, 21, 24, 25, 28, and 29, incorporated herein.

According to any one of the above aspects and embodiments, unless it is specifically stated otherwise, the premix compositions and the food products such as cream cheese, ricotta, ice cream or yogurt are devoid of any animal component or ingredient.

The invention will now be exemplified in the following description of experiments that were carried out in accordance with the invention. It is to be understood that these examples are intended to be in the nature of illustration rather than of limitation. Many modifications and variations of these examples are possible in light of the above teaching. It is therefore to be understood that within the scope of the appended claims, the invention may be practiced otherwise, in a myriad of possible ways, than as specifically described hereinbelow.

### EXAMPLES

### Materials and Methods

The pH was determined using pH meter. Total solids content was determined using loss-on-drying method (LOD). Protein content was determined using Kjeldahl method with a factor conversion of 6.38 for dairy. Oil/Fat content was determined using oil extraction system such as Soxhlet extraction [e.g., SOXTHERM™). Sugar content was determined using HPLC under standard conditions. Salt content was determined using Titration AA method. Organoleptic properties were determined using a testing panel. Texture (max load) (hardness) is determined using a Texture Profile Analyzer with a spindle diameter of 11.1 mm at a speed of 100 mm/min and work depth of 50 mm.

### Example 1. Production of a Recombinant Beta-Lactoglobulin

Pichia pastoris mutant, BG11 (slow methanol utilization derivative (MutS), Alcohol Oxidase (AOX1) aox1Δ) yeast was transformed with a plasmid encoding beta-lactoglobulin (BLG) variant B between AOX1 promoter (originated from P. pastoris) and AOX1 transcription terminator (originated from P. pastoris). The BLG is encoded by the DNA sequence identified as SEQ ID NO:3.

The amino acid sequence of the rBLG, produced from the disclosed transgenic microorganism is identified as SEQ ID NO:4.

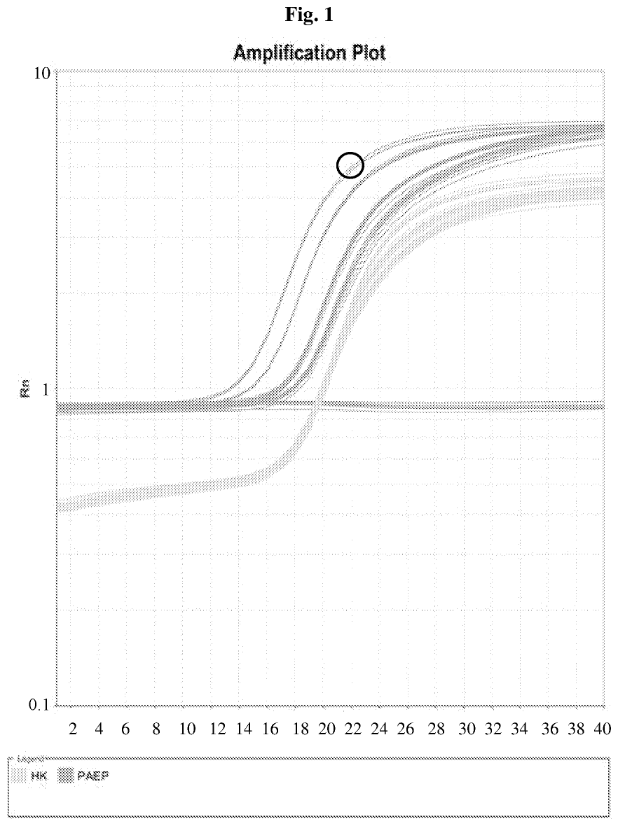

QPCR analysis (FIG. 1) revealed that the (AOX1 promoter)-(rBLG encoding sequence)-(AOX1 transcription terminator) was inserted in the Pichia pastoris genome at the AOX1 promotor site at multiple copies (˜14, 13-15).

Specifically, FIG. 1 shows the presence of House Keeping (HK) gene of Progestagen-associated endometrial protein (PAEP, a homolog of rBLG and thus is used as a marker for rBLG).

The transformed Pichia pastoris BG11 then underwent the fermentation steps as described below.

First, a pre-fermentation seed culture (or “Seed”) was produced. Seed culture medium (0.75 L buffered glycerol complex medium (BMGY) was inoculated with the transformed Pichia pastoris BG11 in a 2 L Seed Culture Vessel. The Vessel was shaken at a Speed of 240 rpm, at 30° C. for about 30 hours.

The seed culture was inoculated to a 15 L Fermentation Medium in a 32 L vessel.

Fermentation Medium (FM22) was composed of KH₂PO₄, (NH₄)₂SO₄, CaSO_{4.2}H₂O, K₂SO₄, MgSO₄·7H₂O, and glycerol.

The culture was agitated at a speed of 300 rpm to 600 rpm, at 30° C., at pH 6.0, maintaining dissolved oxygen (DO) levels at 35%.

During fermentation, cells were fed first continuously with 50% glycerol, and then, when reaching an OD of 200-300, continuously with methanol and PTM1 Trace Salts (no glycerol) to induce expression of rBLG.

PTM1 was composed of cupric sulfate-5H₂O, Sodium iodide, manganese sulfate-H₂O, sodium molybdate-2H₂O, boric acid, cobalt chloride, zinc chloride, ferrous sulfate-7H₂O, Biotin, and sulfuric acid.

After the fermentation was finished (after ˜5 days), the recombinant protein was isolated from the fermentation medium by the following purification steps: first, the supernatant was isolated, then filtered through 0.2 micron membrane for separation of solids, and then concentrated using 3-5 kDa membranes e.g., via diafiltration until the protein reached the concentration of 9-12%.

The resulting solution comprising the rBLG protein was dialyzed against water. Without being limited to any particular theory, it is considered that the dialysis removes components responsible for an aftertaste resulting from the presence of the microorganism. Then, the solution was dried by spray-drying to obtain a powder—rBLG isolate. The isolate was further inspected by LC-MS and HPLC, as described below. As follows from the examination, the isolate comprises at least 85 wt % of rBLG protein, the rest are carbohydrates and moisture.

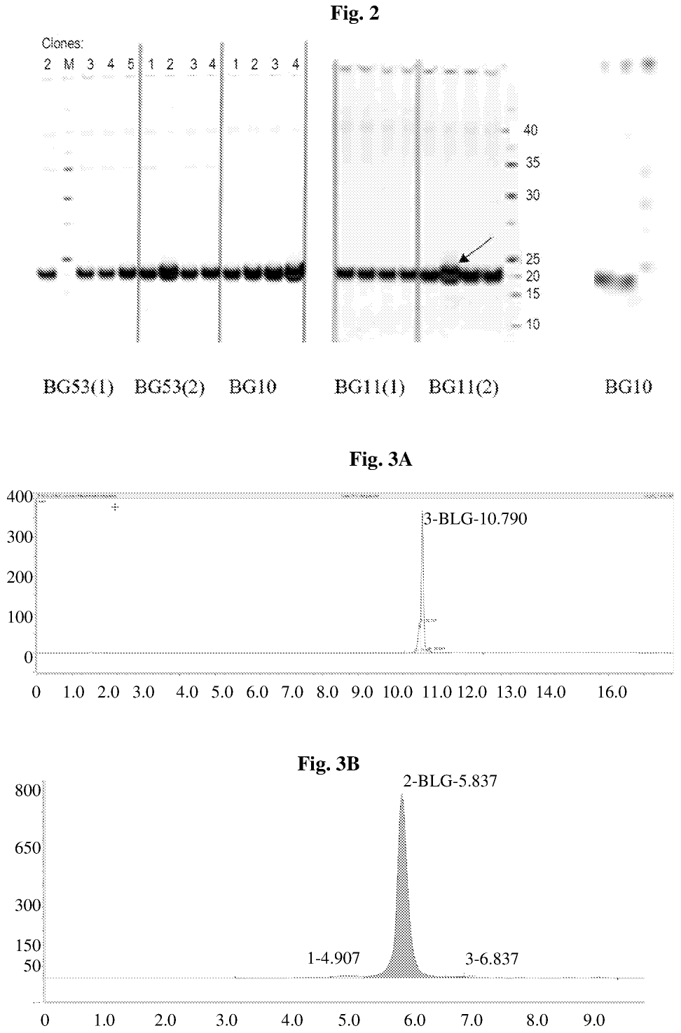

The recombinant beta-lactoglobulin was analyzed by SDS-PAGE gel and further by HPLC and determined to be ˜95% pure (FIG. 2, FIG. 3A and FIG. 3B).

The same experiment was repeated with wild type P. pastoris strain (BG10) and in the strain overexpressing Hac1 and Kar2 chaperones (BG53). All strains were transformed with pJAG plasmid encoding rBLG between AOX1 promoter and transcription terminator and or with pJAG and pJAN plasmids. Apparently, the clone GB11 transformed with pJAG and pJAN plasmids comprising approximately 14 copies of the nucleic acid encoding rBLG provided the highest yield of obtained protein.

FIG. 2 shows that beta-lactoglobulin was successfully expressed in isolates of all three P. pastoris strains (band at ˜18 kDa). However, the best results were received for P. pastoris strains comprising from 13 to 15 copies of beta-lactoglobulin ORFs.

The results support the feasibility of the technology in expressing the protein in low methanol medium.

The protein expressed in BG11 transformed with pJAG and pJAN plasmids (denoted as BG11(2) in FIG. 2 and marked by the arrow), was further analyzed using reverse phase HPLC (FIG. 3A) under the following conditions: HPLC RP-C18 the conditions are:
Column: BioBasic-18, 150×4.6 mm, 5 μm. Cat #72105-154630 (Thermo Scientific).
20 ul of sample using concentration of 10 mg/ml.
Mobile phase: Mobile phase A: 0.05% of TFA in MQW, Mobile phase C: 0.05% of TFA in Acetonitrile.
BLG run time: 10.86 min.

The conditions of the run are described in Table 1.

TABLE 1
Elution using HPLC RP-C18 column

| | | Flow | % | % | % | |
|---|---|---|---|---|---|---|
| No | Time | [ml/min] | B | C | D | Curve |
| 1 | 0.000 | | | | | |
| 2 | 0.000 | 1.200 | 0.0 | 10.0 | 0.0 | 5 |
| 3 | 3.000 | 1.200 | 0.0 | 10.0 | 0.0 | 5 |
| 4 | 12.400 | 1.200 | 0.0 | 64.3 | 0.0 | 5 |
| 5 | 12.500 | 1.200 | 0.0 | 10.0 | 0.0 | 5 |
| 6 | 13.000 | 1.200 | 0.0 | 10.0 | 0.0 | 5 |
| 7 | New row | | | | | |

| | | |
|---|---|---|
| 8 | 13.000 | Stop Run |

In an additional experiment, the rBLG protein was characterized using SEC HPLC utilizing PROTEEMA HPLC Column 150×8 mm PSS+50×8 mm guard and using the following eluent” Phosphate buffer 20 m pH7 100 mM NaCl; about 0.5 μl of the protein sample at the concentration of 1 mg/ml were injected. The results are presented in FIG. 3B. The protein was eluted in a single peak at the retention time of about 5.9 min, with a small shoulder at the retention time of 5.1 and 5.4 mins.

### Example 2. Comparison Analysis

To determine the possibility of producing cheese analogue from recombinant BLG, a traditional method of producing Ricotta was applied onto a composition comprising 5% recombinant betalactoglobuline (rBLG), 25% lipids (shea oil) and minerals (magnesium chloride, calcium chloride, and sodium chloride). Unexpectedly, after homogenization, the coagulation occurred during pasteurization within the pasteurizer. Therefore, a modified method of producing Ricotta has been designed, as disclosed herein.

Table 2 summarizes differences between three methods of production of the cheese analogue products and the result of each stage in the cheese manufacturing process.

Traditional: a composition comprising a total of 5% proteins (4.4% all milk proteins except BLG, 0.6% BLG), 25% lipids, and minerals.

Animal-free composition: 5% rBLG, 25% lipids and minerals

Animal-free and mineral-free composition: 5% rBLG and 25% lipids.

TABLE 2
Cheese manufacturing

| | | Animal Free & |
|---|---|---|
| Traditional | Animal Free | Mineral Free |
| 5% all milk proteins | 5% rBLG + 25% Oil + | 5% rBLG + 25% oil |
| (0.6% BLG) + 25% | coagulation minerals | without coagulation |
| oil + coagulation | | minerals |
| minerals | | |

Homogenization

| | | |
|---|---|---|
| Pasteurization: (with | Pasteurization: (5′ at | Pasteurization: (5′ at |
| subsequent cooling to | 90° C.; cooling to | 90°; cooling to 30°) |
| 30°) | 30° C.) | No coagulation |
| | Coagulation within the | |
| | pasteurizer | |
| | | Addition of |
| | | coagulation minerals |
| | | 1^{st} Coagulation |
| | | (Ricotta cheese analogue |
| | | obtained) |
| Lactic Acidification + | | Lactic Acidification, No |
| Chymosin | | Chymosin |
| Coagulation | | Coagulation |
| Addition of sodium | | Addition of sodium |
| chloride | | chloride |
| Smoothening | | Smoothening |
| Cream Cheese | | Cream cheese analogue |

The results show that when using a high amount of rBLG in the presence of coagulation minerals, pre-mature coagulation takes place.

It is noted that the adding of a low dosage of Chymosin (1/10 of a hard cheese dosage) is typically required to treat casein and prevent casein coagulation. Yet, in the above non-limiting animal-free and mineral-free example, casein is not present and therefore there is no need to add chymosin.

### Example 3. Cream Cheese Analogue Pre-Mix

For the instant production of non-dairy cream cheese analogue using the recombinant beta-lactoglobulin (rBLG) of Example 1, the mixture of Table 3 was prepared in powder form.

TABLE 3
Cream cheese analogue pre-mix components

| Component | % (weight) | % (range) |
|---|---|---|
| Recombinant protein (rBLG) | 41.3 | 40-50 |
| Dextrose (sugar) | 20.2 | 17-22 |
| Etenia 457 (stabilizer) | 38.5 | 30-40 |
| Total | 100.0 | 100 |

ETENIA 457 is a functional enzymatically treated potato starch.

### Example 4. Production of Non-Dairy Cream Cheese Analogue

The cream cheese analogue pre-mix of Example 3 was then processed into a non-dairy cream cheese analogue product by the following procedure:
Integrate within a blender the pre-mixture of Example 3, with water heated to 40° C. to form an aqueous mix.
Agitate the aqueous mix in an insulated tank under slow agitation (30-45 rpm) for more than 45 minutes.
Melt non-animal, natural fat in a separated tank by heating to 40-45° C. using indirect heat.
Integrate the melted fat into the aqueous mix under moderate agitation (50-60 rpm).
Heat, using a plate heat exchanger, to 60° C. and double-stage homogenize at 50 and then at 200 bar (total 250 bar).
Pasteurize to 90° C. for 5 minutes and cool down to 32° C.
Transport into a fermenter under slow agitation (20-30 rpm)
Add calcium chloride and bacterial culture.
Stop agitation.
Ferment for 10-14 hours until reaching pH 4.7.
Add table salt (sodium chloride) under moderate agitation (50-60 rpm).

The components of a non-limiting example of a non-dairy cream cheese are provided in Table 4.

TABLE 4
Non-dairy cream cheese analogue components

| Component | % (weight) | % (range) |
|---|---|---|
| Pre-mixture (content of rBLG) | 10.4 (~4.3) | 9-12 |
| AkoPlanet ™ PBC 110-44 (AAK) - refined | 25 | 20-30 |
| coconut oil (fat) | | |
| NaCl | 0.9 | 0.8-1.2 |
| Potassium sorbate (preservative agent) | 0.1 | |
| Calcium chloride (firming agent) | 0.15 | 0.1-0.2 |
| CHN 19 (Mesophilic lactic acid bacteria) | 1 U/10 L | |
| Water | to 100 | 60-70 |

The nutritional value of the non-dairy cream cheese (in 100 g final product, after fermentation) is provided in Table 5.

TABLE 5
Non-dairy cream cheese analogue nutritional value

| | |
|---|---|
| Energy (Kcal) | 261 |
| Fat (g) | 25 |
| Carbohydrate (g) | 4.5 |
| of which sugars (g) | 0.5 |
| Protein (g) | 4 |
| Sodium (mg) | 360 |
| Calcium (mg) | 42 |

In other examples, a BLG purified from cow milk is used to prepare non-animal-free cream cheese analogue.

The cream cheese analogue can be filled and stored under cold or warm conditions as described below.

### Filling Product to a Final Container:

Optionally add potassium sorbate diluted 1/2 in water at room temperature.
Homogenize at 70 (60-100) bar to a balance tank.
Fill cups at room temperature and cool down rapidly.
Let the non-dairy cream cheese set for 24 hours until the final texture is achieved.

For “warm” filling (filling of hot product to a final container):
Heat to 68° C. using a tubular heat exchanger.
Homogenize at 70 (60-100) bar to an insulated balance tank.
Fill cups at >65° C. and cool down rapidly.
Let the non-dairy cream cheese set for 48 hours until the achievement of the final texture of a cream cheese as defined by Brghenti M. et al. 2008 (“Characterization of the Rheological, Textural, and Sensory Properties of Samples of Commercial US Cream Cheese with Different Fat Contents” J. Dairy Sci. 91:4501-4517), the content of which is incorporated herein by reference in its entirety has been achieved.

### Example 5—Preparing Ricotta Cheese Analogue

To produce animal-free Ricotta cheese analogue using the recombinant beta-lactoglobulin (rBLG) of Example 1, a mixture of components as detailed in Table 6 was prepared according to the following steps:

The recombinant beta-lactoglobulin powder was mixed with water to the initial concentration of 8.16% w/v for 40 min at 40° C. To the water mixture, dextrose and shea oil were added and the mixture was homogenized at 60° C., 200 bar followed by homogenization at 50 bar.

Then, the homogenized mixture was subjected to pasteurization at 90° C., 5 min. After the pasteurization was terminated, the mixture reached a temperature of 32° C.

To the pasteurized product, CaCl₂) was added whereby coagulation took place and Ricotta cheese analogue was thus obtained.

TABLE 6
Ricotta cheese composition

| Ingredient | % | Amount (gr) |
|---|---|---|
| rBLG | 5.40 | 54.00 |
| Sugar (e.g., dextrose) | 2.10 | 21.00 |
| Oil (Shia oil) | 25.00 | 250.00 |
| Calcium Chloride | 0.25 | 2.50 |
| Salt | 0.90 | 9.00 |
| Water | 66.16 | 661.64 |
| Total | 100.00 | 1000.00 |

To verify the production of Ricotta cheese analogue, several parameters were determined as described in Materials and Methods section above and the results are summarized in Table 7.

TABLE 7
Properties of Ricotta cheese analogue

| | |
|---|---|
| pH | 5.8-6.7 |
| Total solids content | 10-27.2% |
| Protein content | 5-10% |
| Oil/Fat content | 3-12% |
| Sugar content | 2-5% |
| Salt content | 0-0.2% |
| Organoleptic properties | milky, slightly sweet |
| Texture | 4-10N |

The expected nutritional values of the Ricotta cheese analogue made by this Example are provided in Table 8 below:

TABLE 8
Nutritional values of the Ricotta cheese analogue
Ricotta Nutritional Value [In 100 g]

| | |
|---|---|
| Energy [Kcal] | 145 |
| Lipids | 13 |
| Carbohydrate | 2 |
| Of which sugars | 2 |
| Protein | 5 |
| Sodium [mg] | 360 |
| Calcium [mg] | 125 |

The resulted cream cheese analogue has the following properties: Appearance: white milky color; Glossiness: matt interior with a shiny surface; Consistency: slightly adhesive, covering the mouth but melt quickly; Structure: dense without air formation; Thickness: able to build up on a spoon; Flavor: mild milky, buttery (not from protein); and Taste: Sweet, Salty, metallic.

In other examples, a BLG purified from cow milk is used to prepare non-animal-free Ricotta cheese analogue.

### Example 6—Preparing Cream Cheese Analogue

To produce a cream cheese analogue, the Ricotta cheese analogue of Example 5 was exposed to lactic acidification with mesophilic culture at 32° C. and the acidification was terminated at pH of 4.5, whereby the second coagulation took place during/after lactic acidification. To the acidified product, table salt was added (0.2-2%) and then smoothing was performed by applying shear forces on the salted product followed by cooling to 4° C., thereby producing cream cheese.

To verify the production of cream cheese, its properties were determined as described in Materials and Method section. The results are summarized in Table 9.

TABLE 9
Cream Cheese analogue properties

| | |
|---|---|
| pH | 4.3-4.8 |
| Total solids content | 23.5-46.2% |
| Protein content | 3-8% |
| Lipid content | 20-35% |
| Sugar content | 0-2% |
| Salt content | 0.5-1.2% |
| Organoleptic properties | milky, cultured, slightly acidic |
| Texture | 6-15(N) |

The resulted cream cheese analogue has the following properties: Appearance: white milky colour; Glossiness: matt interior with a shiny surface; Consistency: slightly adhesive, covering the mouth but melt quickly; Structure: dense without air formation; Thickness: able to build up on a spoon; Flavour: milky, sour, cultured, acetaldehyde, alcohol, buttery (not from proteins); Taste: sour, sweet, salty; and Penetration strength: 80-100[g].

The expected nutritional values of the Cream Cheese analogue made by this Example is provided in Table 10 below:

TABLE 10
Nutritional values of the Cream Cheese analogue
Cream cheese Nutritional Value [In 100 g]

| | |
|---|---|
| Energy [Kcal] | 255 |
| Lipids | 25 |
| Carbohydrate | 2 |
| Of which sugars | 2 |
| Protein | 5 |
| Sodium [mg] | 360 |
| Calcium [mg] | 125 |

In other examples a BLG purified from cow milk is used to prepare non-animal-free Ricotta cheese analogue.

### Example 7. Preparation of Step (Soft) Cream Cheese Analogue

Cream cheese analogue pre-mix composition for preparation of a soft cream cheese analogue was prepared according to Table 11. By using only recombinant BLG the composition is animal-free.

TABLE 11
Pre-mixture composition

| Component | % (weight) | Per 1000 gr | Range % |
|---|---|---|---|
| rBLG 85% | 46.40 | 464.00 | 40-50 |
| Trisodium citrate (chelator of | 1.34 | 13.38 | 1.3-1.7 |
| calcium, mild acidic taste) | | | |
| Dextrose (sugar) | 22.68 | 226.83 | 22-25 |
| Starch (stabilizer) | 29.58 | 295.80 | 29-50 |
| Total | 100.0 | 1000.0 | |

A soft cream cheese analogue was then prepared using the pre-mixture composition described in Table 11 and according to the procedure described below. The content of the soft cream cheese analogue is provided in Table 12. The nutritional value and characterization of the resulted cream cheese analogue are provided in Tables 13 and 14, respectively.

TABLE 12
Cream cheese analogue composition

| Component | % (weight) | % (range) |
|---|---|---|
| Pre-mixture (rBLG) | 10.20 (4.7) | 10-11 |
| Plant oil such as shea oil | 25 | 20-30 |
| NaCl | 0.9 | 0.6-1.2 |
| Calcium Chloride | 0.06 | 0.01-0.1 |
| (firming agent) | | |

| | |
|---|---|
| Mesophilic lactic acid | 1 U/10 L |

| | | |
|---|---|---|
| bacteria | | |
| Water | to 100 | 60-70 |

The cream cheese analogue was prepared as described: Integrate within a blender the pre-mixture of prepared according to Table 11, with water heated to 40° C. to form an aqueous mix. Agitate the aqueous mix in an insulated tank under slow agitation (30-45 rpm) for >45 minutes. Add oil into the aqueous mix under moderate agitation (50-60 rpm). If non-animal natural fat is used melt it in a separated tank to 40-45° C. using indirect heat before adding. Heat, using plate heat exchanger, to 60° C. and double-stage homogenize at 50 and then at 200 bar (total 250 bar). Pasteurize to 85° C. for 2 minutes and cool down to 24-32° C. Transport into a fermenter under slow agitation (20-30 rpm). Add calcium chloride and bacterial culture. Stop agitation. Ferment for 6-14 hours until pH 4.5-4.7 was reached. The table salt was then added under moderate agitation (50-60 rpm). Filling product to final container. Homogenize at 70 (60-100) bar to a balance tank. Fill cups at room temperature and cool down rapidly. Let the cream cheese analogue set for 24 hours until the final texture has been achieved.

For “warm” filling (filling of hot product to the final container): Heat to 68° C. using tubular heat exchanger. Homogenize at 70 (60-100) bar to an insulated balance tank. Fill cups at >65° C. and cool down rapidly. Let the cream cheese analogue set for 48 hours until the final texture is determined to be that of a cream cheese.

TABLE 13
Nutritional value (per 100 g of finished cream cheese analogue)

| | |
|---|---|
| Energy (Kcal) | 255 |
| Fat (g) | 25 |
| Carbohydrate (g) | 3.5 |
| Of which Sugars (g) | 0.5 |
| Protein (g) | 4 |
| Sodium (mg) | 360 |
| Calcium (mg) | 17 |

The resulted cream cheese analogue has the following properties: Appearance: white milky colour; Glossiness: matt interior with a shiny surface; Consistency: slightly adhesive, covering the mouth but melt quickly; Structure: dense without air formation; Thickness: able to build up on a spoon; Flavour: milky, sour, cultured, acetaldehyde, alcohol, buttery (not from proteins); Taste: sour, sweet, salty; and Penetration strength: 80-100[g].

A similar procedure was performed but without adding rBLG. The resulted products were compared.

TABLE 14
Cream cheese analogue characterization

| | With protein | Without protein |
|---|---|---|
| Taste | Cheesy | Not cheesy |
| Aroma | Good | Moderate |
| Coagulation | Coagulate | Didn't coagulate |
| Color (Hunter lab) | L = 92 | L = 71 |
| Spreadability (g*s) (T.A - micro | 3600 | Too low to measure |
| stable/spreadability rig) | | |
| Creaminess | Creamy | Sandy |
| Firmness (g) (T.A - micro stable/ | 3000 | Too low to measure |
| spreadability rig) | | |
| pH | 4.5 | 4.5 |

In other examples a BLG purified from cow milk is used to prepare non-animal-free cream cheese analogue.

### Example 8. Preparation of Firm Cream Cheese Analogues

Premixture composition for preparation of a firm cream cheese analogue was prepared as according to Table 15. The content of the firm cream cheese analogue is provided in Table 16. By using only recombinant BLG the composition is animal-free.

TABLE 15
Pre-mixture firm cream cheese composition

| Component | % (weight) | Per 1000 gr | Range % |
|---|---|---|---|
| rBLG 85% | 38.42 | 384.2 | 35-50 |
| Trisodium citrate | 1.12 | 11.2 | 1-1.5 |
| Dextrose (sugar) | 18.78 | 187.8 | 18-20 |
| Starch (stabilizer) | 40.82 | 408.2 | 35-45 |
| LBG (Lcoust | 0.86 | 8.6 | 0.8-0.9 |
| bean gum, | | | |
| stabilizer) | | | |
| Total | 100.0 | 1000.0 | |

A firm cream cheese analogue was then prepared using the pre-mixture composition according to Table 15 and according to the procedure described below. The nutritional value and characterization of the resulted cream cheese analogue are provided in Tables 17 and 18, respectively.

TABLE 16
Firm cream cheese analogue composition

| Component | % (weight) | % (range) |
|---|---|---|
| Pre-mixture (rBLG) | 12.4 (~4.6) | 12-13 |
| Plant oil | 25 | 20-30 |
| NaCl | 0.9 | 0.6-1.2 |
| Calcium Chloride (firming agent) | 0.06 | 0.01-0.1 |

| | |
|---|---|
| Mesophilic lactic acid bacteria | 1 U/10 L |

| | | |
|---|---|---|
| Water | to 100 | 60-70 |

The firm cream cheese analogue was prepared as described: Integrate within a blender the pre-mixture of prepared according to Table 15, with water heated to 40° C. to form an aqueous mix. Agitate the aqueous mix in an insulated tank under slow agitation (30-45 rpm) for >45 minutes. Add oil into the aqueous mix under moderate agitation (50-60 rpm). If non-animal natural fat is used melt it in a separated tank to 40-45° C. using indirect heat before adding Heat, using plate heat exchanger, to 60° C. and double-stage homogenize at 50 and then at 200 bar (total 250 bar). Pasteurize to 85° C. for 2 minutes and cool down to 24-32° C. Transport into a fermentation under slow agitation (20-30 rpm). Add Calcium Chloride and Bacterial culture. Stop agitation. Ferment for 6-14 hours until pH 4.5-4.7 was reached. The table salt was then added under moderate agitation (50-60 rpm).

Filling product to a final container: Homogenize at 70 (60-100) bar to a balance tank. Fill cups at room temperature and cool down rapidly. Let the cream cheese analogue set for 24 hours until the final texture has been achieved.

For “warm” filling (filling of hot product to the final container): Heat to 68° C. using tubular heat exchanger. Homogenize at 70 (60-100) bar to an insulated balance tank. Fill cups at >65° C. and cool down rapidly. Let the cream cheese analogue set for 48 hours until the final texture is determined to be that of cream cheese.

TABLE 17
Nutritional value (per 100 g of the finished product)

| | |
|---|---|
| Energy (Kcal) | 263 |
| Fat (g) | 25 |
| Carbohydrate (g) | 5.6 |
| Of which Sugars (g) | 0.5 |
| Protein (g) | 4 |
| Sodium (mg) | 360 |
| Calcium (mg) | 17 |

The resulted cream cheese analogue has the following properties: Appearance: white milky colour; Glossiness: matt interior with a shiny surface; Consistency: slightly adhesive, covering the mouth but melt quickly; Structure: dense without air formation; Thickness: able to build up on a spoon; Flavour: milky, sour, cultured, acetaldehyde, alcohol, buttery (not from proteins); Taste: sour, sweet, salty; and Penetration strength: 80-100[g].

A similar procedure was performed but without adding rBLG. The resulted products were compared.

TABLE 18
Firm Cream cheese analogue characterization

| | With protein | Without protein |
|---|---|---|
| Taste | Cheesy | Not cheesy |
| Aroma | Good | Moderate |
| Coagulation | Coagulate | Didn't coagulate |
| Color (Hunter lab) | L = 92 | L = 71 |
| Spreadability (g*s) (T.A - micro stable/ | 4190 | Too low to |
| spreadability rig) | | measure |
| Creaminess | Creamy | Sandy |
| Firmness (g) (T.A - micro stable/ | 3000 | Too low to |
| spreadability rig) | | measure |
| pH | 4.5 | 4.5 |

In other examples, a BLG purified from cow milk is used to prepare non-animal-free cream cheese analogue.

### Example 9. Preparation of Ice Cream Analogues

Premixture composition for preparation of an ice cream analogue is prepared according to Table 19 or Table 20. By using only recombinant BLG the composition is animal-free.

TABLE 19
Ice cream analogue pre-mix composition

| Component | % (weight) | Per 1000 gr |
|---|---|---|
| Dietary fibers - Inulin | 6.21 | 62.1 |
| Protein - rBLG powder (85% protein) | 8.11 | 81.1 |
| Sugar - Dextrose | 20.69 | 206.9 |
| Sugar - sucrose | 41.37 | 413.7 |
| Sugar - 80/38 Glucose syrup | 20.69 | 206.9 |
| Emulsifier - sucrose ester SP70 | 1.03 | 10.3 |
| Mineral - CaCl₂ | 0.34 | 3.4 |
| Thickeners - LBG (Lcoust bean gum) | 0.52 | 5.2 |
| Thickeners - Carrageenan | 0.17 | 1.7 |
| Cream flavor, Edlong #1413271 | 0.86 | 8.6 |
| Color, Vanilla yellow P-WA, Sensient | 0.01 | 0.1 |
| Total | 100 | 1000 |

TABLE 20
Ice cream analogue pre-mix composition - summary

| Mix | % | % Range | 1000 gr |
|---|---|---|---|
| rBLG | 8.18 | 6-13 | 81.8 |
| Sugars | 83.47 | 80-85 | 834.7 |
| Coagulation minerals | 0.35 | 0.3-0.4 | 3.5 |
| Dietary fibers | 6.26 | 6-7 | 62.6 |
| Emulsifier | 1.04 | 1-1.2 | 10.4 |
| Stabilizers | 0.70 | 0.65-0.75 | 7.0 |
| Total | 100.00 | | 1000 |

An ice cream analogue was then prepared using the pre-mixture composition according to Table 20 and according to the procedure described below. The content of the ice cream analogue is provided in Table 21. The nutritional value and characterization of the resulted cream cheese analogue are provided in Tables 22 and 23, respectively.

TABLE 21
Ice cream analogue composition

| Component | % (weight) | % (range) |
|---|---|---|
| Pre-mixture | 29 | 25-35 |
| Plant oil e.g., shea oil | 10 | 5-20 |
| Vanilla extract (optional) | 0.1 | 0-0.5 |
| Water | to 100 | 55-70 |

The non-animal, dairy ice cream analogue was prepared as described: Integrate within a blender the pre-mixture of prepared according to Table 20, with water heated to 40° C. to form an aqueous mix. Agitate the aqueous mix in an insulated tank under slow agitation (30-45 rpm) for >45 minutes. Add oil into the aqueous mix under moderate agitation (50-60 rpm). If non-animal natural fat is used melt it in a separated tank to 40-45° C. using indirect heat before adding. Heat, using plate heat exchanger, to 60° C. and double-stage homogenize at 50 and then at 200 bar (total 250 bar). Pasteurize to 85° C. for 2 minutes and cool down to 4° C. Transport into a balance tank and let it mature for 3-5 hours. Enter the solution to ice cream maker. Store at −18° C.

TABLE 22
Nutritional value (per 100 g of the finished product)

| | |
|---|---|
| Energy (Kcal) | 215 |
| Fat (g) | 22.5 |
| Carbohydrate (g) | 0.6 |
| Of which Sugars (g) | 0.5 |
| Protein (g) | 6.5 |
| Sodium (mg) | 360 |
| Calcium (mg) | 17 |

The resulted ice cream analogue has the following properties: Appearance: white milky colour; Glossiness: shiny surface and interior; Consistency: slightly adhesive, covering the mouth but melt quickly; Structure: Airy but stable at freezing temperature; Thickness: able to build up on a spoon; Flavour: Milky, buttery, and vanilla (not from protein); Taste: Sweet; Overrun: 30-40%.

A similar procedure was performed but without adding rBLG. The resulted products were compared.

TABLE 23
Ice cream analogue characterization

| | With protein | Without protein |
|---|---|---|
| Taste | Milky taste (typical) | Non milky taste |
| Aroma | Good | Good |
| Color (chroma | L = 94.23, a = −0.83, | L = 98.56, a = −0.19, |
| meter) | b = 4.48 | b = 2.71 |
| Density (kg/L) | 0.85 | 0.76 |
| Mouthfeels | Creamy (typical) | Sandy |
| Overrun | 23% | 44% |
| pH | 6.6 | 6.6 |
| Ice crystals | Small ice crystals (typical) | Large Ice crystals |
| Firmness (T.A - | 2333 | 1502 |
| micro stable/p6) | | |

In other examples a BLG purified from cow milk is used to prepare non-animal-free cream cheese analogue.

### Example 10. Preparation of Stirred Yogurt

Premixture composition for preparation of a yogurt analogue is prepared as according to Table 24. By using only recombinant BLG the composition is animal-free.

TABLE 24
Yogurt analogue pre-mixture composition

| Component | % (weight) | Per 1000 gr | Range % |
|---|---|---|---|
| rBLG 85% | 55.71 | 557.1 | 52-70 |
| (protein) | | | |
| Trisodium citrate | 1.50 | 15.0 | 1.2-1.7 |
| Dextrose (sugar) | 41.18 | 411.8 | 36-50 |
| Pectin (stabilizer) | 1.61 | 16.1 | 0-2 |
| Total | 100.0 | 1000.0 | |

A stirred yogurt analogue was then prepared using the pre-mixture composition according to Table 24 and according to the procedure described below. The content of the stirred yogurt is provided in Table 25. The nutritional value and characterization of the resulted cream cheese analogue are provided in Tables 26 and 27, respectively.

TABLE 25
Yogurt analogue composition

| Component | % (weight) | % (range) |
|---|---|---|
| Pre-mixture (rBLG) | 9.71 (~5.4) | 9.5-11 |
| Plant oil | 3.00 | 0-10 |
| Calcium Chloride (firming agent) | 0.07 | 0-0.1 |
| Sucrose | 8.00 | 0-12 |

| | |
|---|---|
| Thermophilic lactic acid bacteria | 1 U/10 L |

| | | |
|---|---|---|
| Water | to 100 | 60-70 |

The stirred yogurt was prepared as described: Integrate within a blender the pre-mixture of prepared according to Table 24, with water heated to 40° C. to form an aqueous mix. Agitate the aqueous mix in an insulated tank under slow agitation (30-45 rpm) for >45 minutes. Add oil into the aqueous mix under moderate agitation (50-60 rpm). If non-animal natural fat is used melt it in a separated tank to 40-45° C. using indirect heat before adding. Heat, using plate heat exchanger, to 60° C. and double-stage homogenize at 50 and then at 200 bar (total 250 bar). Pasteurize to 85° C. for 2 minutes and cool down to 37-45° C. Transport into a fermentation under slow agitation (20-30 rpm). Add Calcium Chloride and bacterial culture. Stop agitation. Ferment for 5-10 hours until reaching pH 3.9-4.4. Add sucrose under moderate agitation (50-60 rpm).

Filling product to final container: Mix under moderate agitation (50-60 rpm) to a balance tank. Fill cups at room temperature and cool down rapidly. Let the animal-free yogurt analogue set for 24 hours until the final texture has been achieved.

TABLE 26
Nutritional value (per 100 g of finished product)

| | |
|---|---|
| Energy (Kcal) | 79.4 |
| Fat (g) | 3 |
| Carbohydrate (g) | 8.65 |
| Of which Sugars (g) | 8.5 |
| Protein (g) | 4.6 |
| Sodium (mg) | 20.3 |
| Calcium (mg) | 79.4 |

The resulted yogurt analogue has the following properties: Appearance: white milky colour; Glossiness: matt interior with a shiny surface; Consistency: slightly adhesive, covering the mouth but melt quickly; Structure: dense without air formation; Thickness: able to build up on a spoon; Flavour: milky, sour, cultured, acetaldehyde, alcohol, buttery (not from proteins); and Taste: sour, sweet, salty.

A similar procedure was performed but without adding rBLG. The resulted products were compared.

TABLE 27
Yogurt analogue characterization

| | With protein | Without protein |
|---|---|---|
| Taste | Yogurt, acidic taste | Sweet water, acidic taste |
| Aroma | Good | Moderate |
| Color | L = 94.09, a = −2.04, | L = 79.22, a = −1.39, |
| | b = 1.67 | b = −0.88 |
| Coagulation | Hard coagulate (typical) | Didn't coagulate |
| Spreadability | Creamy (typical) | Sandy |
| Creaminess | 521.5 (typical) | 21.76 |
| Firmness | −599 | −10 |
| pH | 4.2 (typical) | 4.4 |
| Solids (%) | 19 | 15 |

In other examples a BLG purified from cow milk is used to prepare non-animal-free cream cheese analogue.

### Example 11. Preparation of High-Protein Firm Cream Cheese Analogues

Premixture composition for preparation of a high-protein firm cream cheese analogue is prepared as according to Table 32. The content of high-protein firm cream cheese is provided in Table 33. By using only recombinant BLG the composition is animal-free.

TABLE 28
High-protein firm cream cheese analogue pre-mixture composition

| Component | % (weight) | Per 1000 gr | Range % |
|---|---|---|---|
| rBLG 85% | 75.24 | 752.4 | 70-80 |
| Trisodium citrate | 1.28 | 12.8 | 1.2-1.7 |
| Dextrose (sugar) | 22.49 | 224.9 | 22-24 |
| LBG | 0.99 | 9.9 | 0-1.2 |
| Total | 100.0 | 1000.0 | |

A high protein cream cheese analogue was then prepared using the pre-mixture composition according to Table 28 and according to the procedure described below (Table 29). The nutritional value and characterization of the resulted cream cheese analogue are provided in Tables 30 and 31, respectively.

TABLE 29
High-protein firm cream cheese analogue composition

| Component | % (weight) | % (range) |
|---|---|---|
| Pre-mixture rBLG | 10.16 (7.6) | 12-13 |
| Plant oil such as Shia oil | 22.5 | 20-30 |
| NaCl | 0.9 | 0.8-1.2 |
| Calcium Chloride | 0.06 | 0.05-0.1 |
| (firming agent) | | |

| | |
|---|---|
| Mesophilic lactic acid | 1 U/10 L |

| | | |
|---|---|---|
| bacteria | | |
| Water | to 100 | 60-70 |

High-protein firm cream cheese analogue was prepared as described in Examples 10. The resulted high-protein firm cream cheese analogue was tested for nutrition value and characteristics. The results are provided in Tables 30 and 31.

TABLE 30
Nutritional value (per 100 g of the finished product)

| | |
|---|---|
| Energy (Kcal) | 215 |
| Fat (g) | 22.5 |
| Carbohydrate (g) | 0.6 |
| Of which Sugars (g) | 0.5 |
| Protein (g) | 6.5 |
| Sodium (mg) | 360 |
| Calcium (mg) | 17 |

The resulted firm cream cheese analogue has the following properties: Appearance: white milky colour; Glossiness: matt interior with a shiny surface; Consistency: slightly adhesive, covering the mouth but melt quickly; Structure: dense without air formation; Thickness: able to build up on a spoon; Flavour: milky, sour, cultured, acetaldehyde, alcohol, buttery (not from proteins); and Taste: sour, sweet, salty.

A similar procedure was performed but without adding rBLG. The resulted products were compared.

TABLE 31
High-protein firm cream cheese analogue characterization

| | With protein | Without protein |
|---|---|---|
| Taste | Cheesy (typical) | Not cheesy |
| Aroma | Good | Good |
| Coagulation | Hard | Didn't coagulate |
| | curd(typical) | |
| Color | Typical | Pale |
| Spreadability | Spreadable | Not spreadable |
| | (typical) | |
| Creaminess | Creamy (typical) | Sandy |
| Firmness | Strong (typical) | Watery |
| pH | 4.5 | 4.5 |
| Solids (%) | 33 | 26 |

In other examples a BLG purified from cow milk is used to prepare non-animal-free cream cheese analogue.

### Example 12. Coagulation of rBLG as a Function of Ca²⁺ Concentration

The coagulation of rBLG as factor of rBLG and Ca²⁺ concentrations was tested as follows: Dissolving (i) rBLG protein, and (ii) dextrose (carbon source for mesophilic culture), in distilled water; Pasteurization—85° C. for 1 minute; Cooling; Adding CaCl₂); Acidification by (i) adding mesophilic culture, and (ii) incubating 32° C. over-night; Cooling to 4° C.; Coagulation was registered when detected; Measure of viscosity and colour.

Used equipment: Texture analyser—T.A micro stable: probe p/6, penetration test—5 mm; Colour—Chromameter.

The results are presented in Tables 32-36.

TABLE 32
Coagulation of rBLG

| | BLG | | | | | | | | | |
|---|---|---|---|---|---|---|---|---|---|---|
| Ca⁺² | 0% | 0.5% | 1% | 2% | 3% | 4% | 5% | 6% | 7% | 8% |
| 0% | DNC | AP | AP | CIC | CIC | CIC | CIC | CIC | CIC | CIC |
| 0.0015% | DNC | AP | AP | CDC | CDC | CDC | CDC | CDC | CDC | CDC |
| 0.0075% | DNC | AP | AP | CDC | CDC | CDC | CDC | CDC | CDC | CDC |
| 0.015% | DNC | AP | AP | CDC | CDC | CDC | CDC | CDC | CDC | CDC |
| 0.05% | DNC | AP | AP | CDC | CDC | CDC | CDPAC | CDPAC | CDPAC | CDPAC |
| 0.075% | DNC | AP | AP | CDC | CDC | CDPAC | CDPAC | CDPAC | CDPAC | CDPAC |
| 0.15% | DNC | AP | CDC | CDC | CDC | CDPAC | CDPAC | CDPAC | CDPAC | CDPAC |

DNC—Does not coagulate;
AP—Aggregates and precipitates post acidification;
CDC—Calcium-dependent, post-acidification coagulation (the desired effect);
CIC—Calcium-independent post-acidification coagulation;
CDPAC—Calcium-dependent, pre-acidification coagulation.

TABLE 33
Texture scoring of the composition

| | BLG | | | | | | | | | |
|---|---|---|---|---|---|---|---|---|---|---|
| Ca⁺² | 0% | 0.5% | 1% | 2% | 3% | 4% | 5% | 6% | 7% | 8% |
| 0.0000% | 0 | 0 | 0 | 4.2 | 4.14 | 4.12 | 4.3 | 4.46 | 4.5 | 29.18 |
| 0.0015% | 0 | 0 | 0 | 4.24 | 4.28 | 5.96 | 8.63 | 9.85 | 11.08 | 36 |
| 0.0075% | 0 | 0 | 0 | 4.15 | 4.15 | 7.92 | 15.08 | 25.69 | 27.04 | 69.35 |
| 0.0150% | 0 | 0 | 0 | 4.13 | 4.87 | 20.3 | 34.19 | 66.67 | 94.73 | 130.22 |
| 0.0500% | 0 | 0 | 0 | 4.11 | 4.16 | 4 | 11.67 | 4.27 | 85.59 | 57.98 |
| 0.0750% | 0 | 0 | 0 | 4.26 | 4.19 | 4.15 | 12.03 | 8.75 | 4.18 | 76.76 |
| 0.1500% | 0 | 0 | 4.12 | 4.46 | 4.07 | 4.07 | 4.07 | 4.16 | 8.14 | 9.27 |

Texture score:
0 = Liquid;
4-5 = Viscous liquid (beverages, drinkable yogurt, milk, ice cream);
6 or more = Gel (cream cheese, spreadable cheese, stirred and set yogurt, semi-hard cheese, desserts)

As can be seen, calcium is not always required for coagulation of rBLG at concentrations of 2 wt % and more upon acidification. However, coagulation of from 2 wt % to 7 wt % of rBLG without calcium forms viscous liquid composition. Only at high concentration of rBLG, i.e., 8 wt % and more, a more viscous product is obtained. Addition of calcium to rBLG allows obtaining viscous products at much lower concentrations of rBLG. Combination of too high concentration of Ca²⁺ and rBLG cause an undesired premature coagulation of rBLG.

TABLE 34
Colour results (L)

| | BLG | | | | | | | |
|---|---|---|---|---|---|---|---|---|
| Ca⁺² | 1% | 2% | 3% | 4% | 5% | 6% | 7% | 8% |
| 0% | | | | | | | | 64.2 |
| 0.0015% | | 60.49 | 66.19 | 62.08 | 65.76 | 60.16 | 68.5 | 70.38 |
| 0.0075% | | 54.47 | 67.13 | 61.42 | 69.26 | 70.99 | 71.27 | 73.82 |
| 0.015% | | 62.35 | 67.73 | 65.53 | 65.51 | 71.78 | 67.55 | 70.65 |
| 0.05% | | 66.88 | 72.26 | 73.13 | | | | |
| 0.075% | | 67.42 | 72.14 | | | | | |
| 0.15% | 49.13 | 66.06 | 71.37 | | | | | |

Colour was measured using the L*a*b* diagram as well known in which a spherical colour solid L* indicates lightness, and a* and b* are the chromaticity coordinates.

TABLE 35
Colour results (a)

| | BLG | | | | | | | |
|---|---|---|---|---|---|---|---|---|
| Ca⁺² | 1% | 2% | 3% | 4% | 5% | 6% | 7% | 8% |
| 0% | | | | | | | | −1.35 |
| 0.0015% | | −1.15 | −2.21 | −2.15 | −1.45 | −1.85 | −1.18 | −1.05 |
| 0.0075% | | −2.04 | −1.86 | −2.37 | −1.18 | −1.08 | −1.04 | −0.86 |
| 0.015% | | −1.6 | −2.09 | −1.46 | −1.37 | −1.02 | −1.42 | −1.04 |
| 0.05% | | −1.26 | −1.24 | −1.35 | | | | |
| 0.075% | | −1.19 | −1.27 | | | | | |
| 0.15% | −0.96 | −1.23 | −1.31 | | | | | |

TABLE 36
Colour results (b)

| | BLG | | | | | | | |
|---|---|---|---|---|---|---|---|---|
| Ca⁺² | 1% | 2% | 3% | 4% | 5% | 6% | 7% | 8% |
| 0% | | | | | | | | 2.9 |
| 0.0015% | | −3.09 | −2.39 | −2.96 | 0.57 | −1.11 | 2.35 | 3.26 |
| 0.0075% | | 6.51 | −1.96 | −3.49 | 1.68 | 2.55 | 2.97 | 3.67 |
| 0.015% | | −3.18 | −1.77 | −1.63 | 0.36 | 2.67 | 1.58 | 2.86 |
| 0.05% | | 0.25 | 1.45 | 1.44 | | | | |
| 0.075% | | 0.5 | 1.52 | | | | | |
| 0.15% | −1.96 | 0.29 | 1.48 | | | | | |

### Example 13. Survey of Evaluating the Dairy Analogue Products

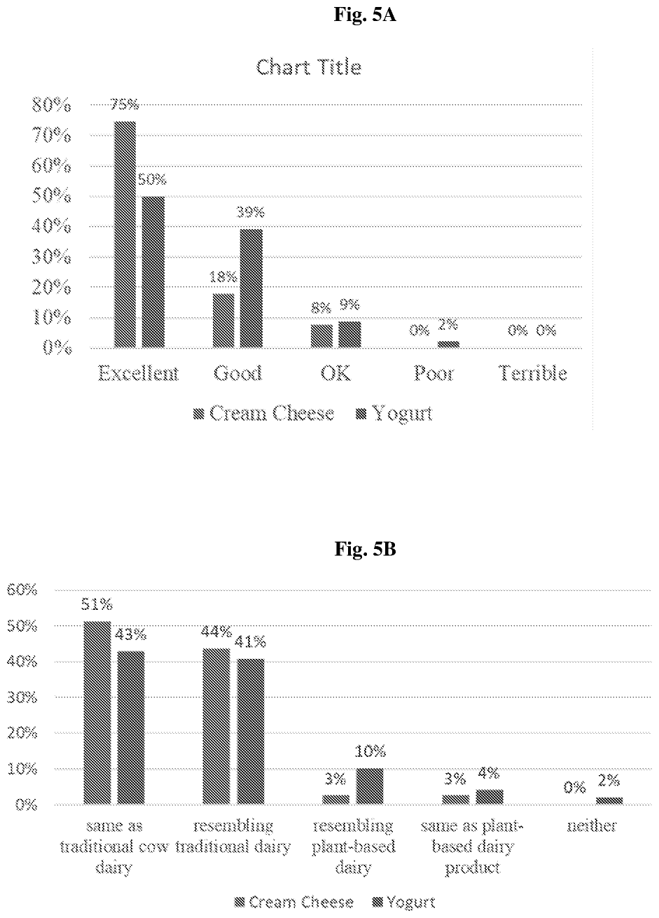

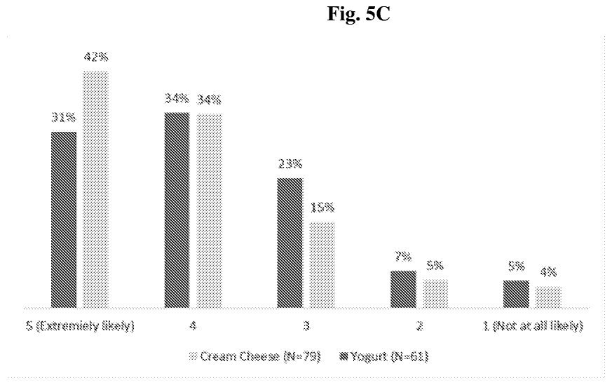

A survey to elucidate client's opinion of the cream cheese and yogurt analogue was conducted. About 80 replies were received from random tasters. The random tasters were asked to evaluate the products from 1 (terrible) to 5 (excellent). The statistics on the random tasters' age and dietary preferences are provided in FIG. 4A and FIG. 4B. The results of the survey are presented in FIG. 5A-5C.

According to the survey, more than 90% responders evaluated the cream cheese analogue as good or excellent with an average of 4.7 and about 90% rated the yogurt as good or excellent with an average of 4.7 (FIG. 5A). More than 95% or about 84% categorize the cream cheese analogue and the yogurt analogues, respectively, as same or resembling to traditional dairy product (FIG. 5B). More than 75% of random tasters responded that they are ready to replace their cream cheese current product with that of the present invention one and more that 65% of random tasters responded similarly regarding the yogurt analogue (FIG. 5C). Interestingly, younger responders were much more enthusiastic replacing products they use by those produced of the present invention. The same is also correct to Vegans and vegetarians. Overall, most of the random tasters liked the dairy-products substitutes of the present invention and expressed their will to replace their current products with those prepared according to the teaching of the present invention.

This application claims priority benefit of U.S. Provisional Patent Application Nos. 63/187,798 filed May 12, 2021, 63/244,133, filed Jul. 21, 2021, and 63/289,640 filed Dec. 15, 2022, which are hereby incorporated by reference in their entirety.

Although the present invention has been described hereinabove by way of preferred embodiments thereof, it can be modified, without departing from the spirit and nature of the subject invention as defined in the appended claims.

1-5. (canceled)

6. A method of preparing a dairy analogue food product comprising a coagulated beta lactoglobulin B (BLG), the method comprising:
(i) pasteurizing a composition comprising a BLG protein, wherein the composition is substantially devoid of coagulation minerals;
(ii) optionally, cooling down the composition of step (i); and
(iii) adding a coagulation mineral salt to the composition of step (ii) or to the composition of step (i), if step (ii) is absent, thereby coagulating the BLG protein,
wherein BLG constitutes at least 51 wt % of the total milk-protein content of the dairy analogue food product, and
wherein the dairy analogue food product comprises at least about 0.75 wt % BLG.

7. The method according to claim 6, wherein the coagulation mineral comprises one or more salts of a mineral selected from the group consisting of calcium, magnesium, phosphorus, potassium, selenium, and zinc.

8. The method according to claim 7, wherein the coagulation mineral salt is calcium chloride.

9. The method according to claim 8, wherein the resulted dairy analogue food product comprises from about 0.75 to about 8 wt % of BLG.

10. The method according to claim 9, wherein the resulted dairy analogue food product comprises from about 0.0015 to about 0.25 wt % of coagulation mineral.

11-13. (canceled)

14. A method of preparing a dairy analogue food product comprising a coagulated BLG protein, the method comprising:
(i) pasteurizing a composition comprising a BLG protein, wherein the composition is substantially devoid of coagulation minerals;
(ii) optionally, cooling down the composition of step (i); and
(iii) acidifying the composition of step (ii) or of step (i), if step (ii) is absent, thereby coagulating the BLG protein;
wherein BLG constitutes at least 51 wt % of the total milk-protein content of the dairy analogue food product, and
wherein the dairy analogue food product comprises at least about 2 wt % BLG.

15. The method according to claim 14, wherein the resulted dairy analogue food product comprises from about 1.5 to about 8 wt % of BLG.

16-26. (canceled)

27. A cream cheese analogue, comprising:
(i) from about 3 to about 10 wt % BLG;
(ii) from about 1 to about 5 wt % of a sugar;
(iii) from about 0 to about 5 wt % of a stabilizer;
(iv) from about 15 to about 35 wt % of a lipid;
(v) from about 0.01 to about 3 wt % of a coagulation mineral;
(vi) optionally, from about 0.5 to about 1.5 wt % of a flavoring salt;
(vii) optionally, from about 0.08 to about 0.5 wt % of a coagulation mineral chelator; and
(viii) water up to 100 wt %;
wherein BLG constitutes at least 51 wt % of the total milk-protein content of the cream cheese analogue.

28. The cream cheese analogue according to claim 27, comprising:
(i) from about 4 to about 8 wt % BLG;
(ii) from about 1 to about 3 wt % of a sugar;
(iii) from about 0.1 to about 4 wt % of a stabilizer;
(iv) from about 20 to about 30 wt % of a lipid;
(v) from about 0.05 to about 2.5 wt % of a coagulation mineral;
(vi) from about 0.5 to about 1.5 wt % of a flavoring salt; and
(vii) water up to 100 wt %

29. The cream cheese analogue according to claim 28, having at least one organoleptic and/or rheologic property of a corresponding dairy cream cheese selected from the group consisting of appearance, glossiness, consistency, structure, thickness, flavor, taste, and penetration strength.

30. The cream cheese analogue according to claim 29, having the appearance, glossiness, consistency, structure, thickness, flavor, taste, and penetration strength of a corresponding dairy cream cheese.

31-39. (canceled)

40. A yogurt analogue, comprising:
(i) from about 2 to about 8 wt % BLG;
(ii) optionally, from about 0.08 to about 0.5 wt % of a coagulation mineral chelator;
(iii) from about 2 to about 15 wt % of a sugar;
(iv) from about 0.05 to about 0.3 wt % of a stabilizer;
(v) from about 1 to about 6 wt % of a lipid;
(vi) from about 0.01 to about 0.15 wt % of a coagulation mineral; and
(vii) water up to 100 wt %,
wherein BLG constitutes at least 51 wt % of the total milk-protein content of the yogurt analog.

41. The yogurt analogue according to claim 39 or claim 40, having at least one organoleptic and/or rheologic property of a corresponding dairy yogurt selected from the group consisting of appearance, glossiness, consistency, structure, thickness, flavor, and taste.

42. The yogurt analogue according to claim 41, having the appearance, glossiness, consistency, structure, thickness, flavor, and taste of a corresponding dairy yogurt.

43-49. (canceled)
